SEQUENCE LISTING (186 sequences)

SEQ ID NO: 1 - protein, 5 aa, Homo Sapiens
KLDFK

SEQ ID NO: 2 - protein, 4 aa, Homo Sapiens
KLDF

SEQ ID NO: 3 - protein, 4 aa, Homo Sapiens
LDFK

SEQ ID NO: 4 - protein, 8 aa, Artificial Sequence
  Synthetic Construct
CGKLDFKG

SEQ ID NO: 5 - protein, 9 aa, Artificial Sequence
  Synthetic Construct
CGKLDFKGG

SEQ ID NO: 6 - protein, 9 aa, Artificial Sequence
  Synthetic Construct
CGGKLDFKG

SEQ ID NO: 7 - protein, 7 aa, Artificial Sequence
  Synthetic Construct
CGKLDFG

SEQ ID NO: 8 - protein, 8 aa, Artificial Sequence
  Synthetic Construct
CGKLDFGG

SEQ ID NO: 9 - protein, 9 aa, Artificial Sequence
  Synthetic Construct
CGKLDFGGG

SEQ ID NO: 10 - protein, 10 aa, Artificial Sequence
  Synthetic Construct
CGKLDFGGGG

SEQ ID NO: 11 - protein, 8 aa, Artificial Sequence
  Synthetic Construct
CGGKLDFG

SEQ ID NO: 12 - protein, 9 aa, Artificial Sequence
  Synthetic Construct
CGGKLDFGG

SEQ ID NO: 13 - protein, 10 aa, Artificial Sequence
  Synthetic Construct
CGGKLDFGGG

SEQ ID NO: 14 - protein, 11 aa, Artificial Sequence
  Synthetic Construct
CGGKLDFGGGG

SEQ ID NO: 15 - protein, 9 aa, Artificial Sequence
  Synthetic Construct
CGGGKLDFG

SEQ ID NO: 16 - protein, 10 aa, Artificial Sequence
  Synthetic Construct
CGGGKLDFGG

SEQ ID NO: 17 - protein, 11 aa, Artificial Sequence
  Synthetic Construct
CGGGKLDFGGG

SEQ ID NO: 18 - protein, 12 aa, Artificial Sequence
  Synthetic Construct
CGGGKLDFGGGG

SEQ ID NO: 19 - protein, 10 aa, Artificial Sequence
  Synthetic Construct
CGGGGKLDFG

SEQ ID NO: 20 - protein, 11 aa, Artificial Sequence
  Synthetic Construct
CGGGGKLDFGG

SEQ ID NO: 21 - protein, 12 aa, Artificial Sequence
  Synthetic Construct
CGGGGKLDFGGG

SEQ ID NO: 22 - protein, 13 aa, Artificial Sequence
  Synthetic Construct
CGGGGKLDFGGGG

SEQ ID NO: 23 - protein, 16 aa, Homo Sapiens
VEVKSEKLDFKDRVQS

SEQ ID NO: 24 - protein, 8 aa, Homo Sapiens
EKLDFKDR

SEQ ID NO: 25 - protein, 7 aa, Homo Sapiens
KLDFKDR

SEQ ID NO: 26 - protein, 10 aa, Homo Sapiens
SEKLDFKDRV

SEQ ID NO: 27 - protein, 8 aa, Artificial Sequence
  Synthetic Construct
CGKLDFKG

SEQ ID NO: 28 - protein, 9 aa, Artificial Sequence
  Synthetic Construct
CGKLDFKGG

SEQ ID NO: 29 - protein, 10 aa, Artificial Sequence
  Synthetic Construct
CGKLDFKGGG

SEQ ID NO: 30 - protein, 11 aa, Artificial Sequence
  Synthetic Construct
CGKLDFKGGGG

SEQ ID NO: 31 - protein, 9 aa, Artificial Sequence
  Synthetic Construct
CGGKLDFKG

SEQ ID NO: 32 - protein, 10 aa, Artificial Sequence
  Synthetic Construct
CGGKLDFKGG

SEQ ID NO: 33 - protein, 11 aa, Artificial Sequence
  Synthetic Construct
CGGKLDFKGGG

SEQ ID NO: 34 - protein, 12 aa, Artificial Sequence
  Synthetic Construct
CGGKLDFKGGGG

SEQ ID NO: 35 - protein, 10 aa, Artificial Sequence
  Synthetic Construct
CGGGKLDFKG

SEQ ID NO: 36 - protein, 11 aa, Artificial Sequence
  Synthetic Construct
CGGGKLDFKGG

SEQ ID NO: 37 - protein, 12 aa, Artificial Sequence
  Synthetic Construct
CGGGKLDFKGGG

SEQ ID NO: 38 - protein, 13 aa, Artificial Sequence
  Synthetic Construct
CGGGKLDFKGGGG

SEQ ID NO: 39 - protein, 11 aa, Artificial Sequence
  Synthetic Construct
CGGGGKLDFKG

SEQ ID NO: 40 - protein, 12 aa, Artificial Sequence
  Synthetic Construct
CGGGGKLDFKGG

SEQ ID NO: 41 - protein, 13 aa, Artificial Sequence
  Synthetic Construct
CGGGGKLDFKGGG

SEQ ID NO: 42 - protein, 14 aa, Artificial Sequence
  Synthetic Construct
CGGGGKLDFKGGGG

SEQ ID NO: 43 - protein, 4 aa, Artificial Sequence
  Synthetic Construct
GCGG

SEQ ID NO: 44 - protein, 5 aa, Artificial Sequence
  Synthetic Construct
GCGGG

SEQ ID NO: 45 - protein, 6 aa, Artificial Sequence
  Synthetic Construct
GGCGGG

SEQ ID NO: 46 - protein, 5 aa, Artificial Sequence
  Synthetic Construct
GGGCG

SEQ ID NO: 47 - protein, 6 aa, Artificial Sequence
  Synthetic Construct
GCGGGG

SEQ ID NO: 48 - protein, 7 aa, Artificial Sequence
  Synthetic Construct
CGLDFKG

SEQ ID NO: 49 - protein, 8 aa, Artificial Sequence
  Synthetic Construct
CGLDFKGG

SEQ ID NO: 50 - protein, 9 aa, Artificial Sequence
  Synthetic Construct
CGLDFKGGG

SEQ ID NO: 51 - protein, 10 aa, Artificial Sequence
  Synthetic Construct
CGLDFKGGGG

SEQ ID NO: 52 - protein, 8 aa, Artificial Sequence
  Synthetic Construct
CGGLDFKG

SEQ ID NO: 53 - protein, 9 aa, Artificial Sequence
  Synthetic Construct
CGGLDFKGG

SEQ ID NO: 54 - protein, 10 aa, Artificial Sequence
  Synthetic Construct
CGGLDFKGGG

SEQ ID NO: 55 - protein, 11 aa, Artificial Sequence
  Synthetic Construct
CGGLDFKGGGG

SEQ ID NO: 56 - protein, 9 aa, Artificial Sequence
  Synthetic Construct
CGGGLDFKG

SEQ ID NO: 57 - protein, 10 aa, Artificial Sequence
  Synthetic Construct
CGGGLDFKGG

SEQ ID NO: 58 - protein, 11 aa, Artificial Sequence
  Synthetic Construct
CGGGLDFKGGG

SEQ ID NO: 59 - protein, 12 aa, Artificial Sequence
  Synthetic Construct
CGGGLDFKGGGG

SEQ ID NO: 60 - protein, 10 aa, Artificial Sequence
  Synthetic Construct
CGGGGLDFKG

SEQ ID NO: 61 - protein, 11 aa, Artificial Sequence
  Synthetic Construct
CGGGGLDFKGG

SEQ ID NO: 62 - protein, 12 aa, Artificial Sequence
  Synthetic Construct
CGGGGLDFKGGG

SEQ ID NO: 63 - protein, 13 aa, Artificial Sequence
  Synthetic Construct
CGGGGLDFKGGGG

SEQ ID NO: 64 - protein, 11 aa, Artificial Sequence
  Synthetic Construct
CGGGKLDFGGC

SEQ ID NO: 65 - protein, 7 aa, Artificial Sequence
  Synthetic Construct
GGGGCGG

SEQ ID NO: 66 - protein, 8 aa, Artificial Sequence
  Synthetic Construct
GGGGCGGG

SEQ ID NO: 67 - protein, 8 aa, Artificial Sequence
  Synthetic Construct
GGGCGGGG

SEQ ID NO: 68 - protein, 8 aa, Artificial Sequence
  Synthetic Construct
GGCGGGGG

SEQ ID NO: 69 - protein, 8 aa, Artificial Sequence
  Synthetic Construct
GCGGGGGG

SEQ ID NO: 70 - protein, 7 aa, Artificial Sequence
  Synthetic Construct
GGGCGGG

SEQ ID NO: 71 - protein, 7 aa, Artificial Sequence
  Synthetic Construct
GGCGGGG

SEQ ID NO: 72 - protein, 7 aa, Artificial Sequence
  Synthetic Construct
GCGGGGG

SEQ ID NO: 73 - protein, 6 aa, Artificial Sequence
  Synthetic Construct
GGGCGG

SEQ ID NO: 74 - protein, 9 aa, Artificial Sequence
  Synthetic Construct
GGGGCGGGG

SEQ ID NO: 75 - protein, 113 aa, Mus Musculus
EVQLQQSGAELVKPGASVKLSCTASGFNIKDTHMHWVKQRPEQGLEWIGKIDPSNGNTQYDPKFQGKATITADTSSNTAYLQLSSLTSEDTAVYYCATGFAYWGQGTLVTVSA

SEQ ID NO: 76 - protein, 107 aa, Mus Musculus
DIQMTQSPASLSASVGETVTITCRASGNIHNYLAWYQQKQGKSPQLLVYNAKTLADGVPSRFSGSGSGTQYSLKINSLQPEDFGSYYCQHFWYTPWTFGGGTKLEIK

SEQ ID NO: 77 - protein, 120 aa, Mus Musculus
QVQLQQSGAELVRPGSSVKISCKASGYAFSSYWMNWVKQRPGQGLEWIGQIYPGDGDTNYNGKFKGKATLTADKSSSTAYMQLSSLTSEDSAVYLCASQIYDGYYTFTYWGQGTLVTVSA

SEQ ID NO: 78 - protein, 112 aa, Mus Musculus
DIVMSQSPSSLAVSAGEKVTMSCKSSQSLLNSRTRKNYLAWYQQKPGQSPKLLIYWASTRVSGVPDRFTGSGSGTDFTLTISSVQAEDLAVYYCKQSYNLWTFGGGTKLEIK

SEQ ID NO: 79 - protein, 119 aa, Mus Musculus
QIQLVQSGPELKKPGETVKISCKASGYTFTNYGMNWVKQAPGKGLKWMGWINTYSGEPTYVDDFKGRFAFSLETSASTAYLQINNLKNEDMATYFCARSPGAYYTLDYWGQGTSVTVSS

SEQ ID NO: 80 - protein, 112 aa, Mus Musculus
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLYTFGGGTKLEIK

SEQ ID NO: 81 - protein, 119 aa, Mus Musculus
QIQLVQSGPELKKPGETVKISCKASGYTFTNYGMNWVKQAPGKGLKWMGWINTYTGEPTYTDDFKGRFAFSLETSASTAYLQINNLKNEDTATYFCGRGIRDYYTMDYWGQGTSVTVSS

SEQ ID NO: 82 - protein, 112 aa, Mus Musculus
DIVMSQSPSSLAVSAGEKVTMSCKSSQSLLNNRTRKNYLAWYQQKPGQSPKLLIYWASTRESGVPDRFTGSGSGTDFTLTISSVQAEDLAVYYCKQSYNLYTFGGGTKLEIK

SEQ ID NO: 83 - protein, 120 aa, Mus Musculus
DVQVQESGPGLVKPSQSLSLTCTVTGYSITSDYAWTWIRQFPGNKLEWMGYISYSGSTSYNPSLKSRLSITRDTSKNQFFLQLNSVTTEDTATYYCAAYYRYGLAYFAYWGQGTLVTVSA

SEQ ID NO: 84 - protein, 112 aa, Mus Musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 85 - protein, 119 aa, Mus Musculus
QIQLVQSGPELKKPGETVKISCKASGYTFTNFGMNWVKQAPGKGLKWMGWINTFTGEPTYVDDFKGRFAFSLETSATTAYLQINNLKNEDTATYFCARSPGRVYTLDYWGQGTSVTVSS

SEQ ID NO: 86 - protein, 112 aa, Mus Musculus; identical to SEQ ID NO: 80

SEQ ID NO: 87 - protein, 114 aa, Mus Musculus
EVQLQQSGTVLARPGASVKMSCKASGYRFTSYWMYWVKQRPGQGLEWIGAIYPGNSDTIYNQRFKGKATLTAVTSASTAYMELSSLANEDSAVYFCTRPYFDSWGQGTTLTVSS

SEQ ID NO: 88 - protein, 112 aa, Mus Musculus; identical to SEQ ID NO: 84

SEQ ID NO: 89 - protein, 120 aa, Mus Musculus
DVQLQESGPGLVKPSQSLSLTCTVTGFSITSDYAWNWIRQFPGNKLEWMGFIRYSGNTRFNPSLKGRGSITRDTSKNQFFLQLNSVTTEDTATYYCASTLEDSYWYFDVWGAGTTVTVSS

SEQ ID NO: 90 - protein, 112 aa, Mus Musculus
DVLMTQTPLSLPVSLGDQASISCRSSQSIVHTNGNTYLEWYLQKPGQSPKLLIYKVSNRFSGVPDRFSGSGSGTDFTLKISRVEAEDLGVYYCFQGSHVPLTFGAGTKLELK

SEQ ID NO: 91 - protein, 112 aa, Mus Musculus
QVQLQQSGAELVKPGASVKLSCKASGYTFTSYYMFWVKQRPGQGLEWIGEINPSNGGSNFNEKFKSKATLTVDKSSSTAYMQLSSLTSEDSAVYYCTRGAFWGQGTLVTVSA

SEQ ID NO: 92 - protein, 112 aa, Mus Musculus
DVVMTQTPLTLSVTIGQPASISCKSSQSLLDSDRKTYLNWLLQRPGQSPKRLIYLVSKLDSGVPDRFTGSGSGTDFTLKISRVEAEDLGVYYCWQVTHFPHTFGAGTKLELK

SEQ ID NO: 93 - protein, 126 aa, Mus Musculus
QVTLKESGPGILKPSQTLSLTCSFSGFSLSTSGMGVGWIRQPSGKGLEWLAHIWWDDDKYYNPSLKNRLTISKDTSRNQVFLKITSVDTADTATYYCVRSIYYYDSSPYYYVMDYWGQGTSVTVSS

SEQ ID NO: 94 - protein, 107 aa, Mus Musculus
DIVMTQSHKFMSTSVGDRVSITCKASQDVSIAVAWYQQKPGQSPKLLIYSASYRNTGVPDRFTGSGSGTDFTFTISSVQAEDLAVYYCQQHYSSPLTFGAGTKLELK

SEQ ID NO: 95 - protein, 8 aa, Mus Musculus
GFNIKDTH

SEQ ID NO: 96 - protein, 8 aa, Mus Musculus
IDPSNGNT

SEQ ID NO: 97 - protein, 6 aa, Mus Musculus
ATGFAY

SEQ ID NO: 98 - protein, 6 aa, Mus Musculus
GNIHNY

SEQ ID NO: 99 - protein, 3 aa, Mus Musculus
NAK

SEQ ID NO: 100 - protein, 9 aa, Mus Musculus
QHFWYTPWT

SEQ ID NO: 101 - protein, 8 aa, Mus Musculus
GYAFSSYW

SEQ ID NO: 102 - protein, 8 aa, Mus Musculus
IYPGDGDT

SEQ ID NO: 103 - protein, 13 aa, Mus Musculus
ASQIYDGYYTFTY

SEQ ID NO: 104 - protein, 12 aa, Mus Musculus
QSLLNSRTRKNY

SEQ ID NO: 105 - protein, 3 aa, Mus Musculus
WAS

SEQ ID NO: 106 - protein, 8 aa, Mus Musculus
KQSYNLWT

SEQ ID NO: 107 - protein, 8 aa, Mus Musculus
GYTFTNYG

SEQ ID NO: 108 - protein, 8 aa, Mus Musculus
INTYSGEP

SEQ ID NO: 109 - protein, 12 aa, Mus Musculus
ARSPGAYYTLDY

SEQ ID NO: 110 - protein, 12 aa, Mus Musculus
QSLLNSRTRKNY

SEQ ID NO: 111 - protein, 3 aa, Mus Musculus
WAS

SEQ ID NO: 112 - protein, 8 aa, Mus Musculus
KQSYNLYT

SEQ ID NO: 113 - protein, 8 aa, Mus Musculus
GYTFTNYG

SEQ ID NO: 114 - protein, 8 aa, Mus Musculus
INTYTGEP

SEQ ID NO: 115 - protein, 12 aa, Mus Musculus
GRGIRDYYTMDY

SEQ ID NO: 116 - protein, 12 aa, Mus Musculus
QSLLNNRTRKNY

SEQ ID NO: 117 - protein, 3 aa, Mus Musculus
WAS

SEQ ID NO: 118 - protein, 8 aa, Mus Musculus
KQSYNLYT

SEQ ID NO: 119 - protein, 9 aa, Mus Musculus
GYSITSDYA

SEQ ID NO: 120 - protein, 7 aa, Mus Musculus
ISYSGST

SEQ ID NO: 121 - protein, 13 aa, Mus Musculus
AAYYRYGLAYFAY

SEQ ID NO: 122 - protein, 11 aa, Mus Musculus
QSLLDSDGKTY

SEQ ID NO: 123 - protein, 3 aa, Mus Musculus
LVS

SEQ ID NO: 124 - protein, 9 aa, Mus Musculus
WQGTHFPQT

SEQ ID NO: 125 - protein, 8 aa, Mus Musculus
GYTFTNFG

SEQ ID NO: 126 - protein, 8 aa, Mus Musculus
INTFTGEP

SEQ ID NO: 127 - protein, 12 aa, Mus Musculus
ARSPGRVYTLDY

SEQ ID NO: 128 - protein, 12 aa, Mus Musculus
QSLLNSRTRKNY

SEQ ID NO: 129 - protein, 3 aa, Mus Musculus
WAS

SEQ ID NO: 130 - protein, 8 aa, Mus Musculus
KQSYNLYT

SEQ ID NO: 131 - protein, 8 aa, Mus Musculus
GYRFTSYW

SEQ ID NO: 132 - protein, 8 aa, Mus Musculus
IYPGNSDT

SEQ ID NO: 133 - protein, 7 aa, Mus Musculus
TRPYFDS

SEQ ID NO: 134 - protein, 11 aa, Mus Musculus
QSLLDSDGKTY

SEQ ID NO: 135 - protein, 3 aa, Mus Musculus
LVS

SEQ ID NO: 136 - protein, 9 aa, Mus Musculus
WQGTHFPQT

SEQ ID NO: 137 - protein, 9 aa, Mus Musculus
GFSITSDYA

SEQ ID NO: 138 - protein, 7 aa, Mus Musculus
IRYSGNT

SEQ ID NO: 139 - protein, 13 aa, Mus Musculus
ASTLEDSYWYFDV

SEQ ID NO: 140 - protein, 11 aa, Mus Musculus
QSIVHTNGNTY

SEQ ID NO: 141 - protein, 3 aa, Mus Musculus
KVS

SEQ ID NO: 142 - protein, 9 aa, Mus Musculus
FQGSHVPLT

SEQ ID NO: 143 - protein, 8 aa, Mus Musculus
GYTFTSYY

SEQ ID NO: 144 - protein, 8 aa, Mus Musculus
INPSNGGS

SEQ ID NO: 145 - protein, 5 aa, Mus Musculus
TRGAF

SEQ ID NO: 146 - protein, 11 aa, Mus Musculus
QSLLDSDRKTY

SEQ ID NO: 147 - protein, 3 aa, Mus Musculus
LVS

SEQ ID NO: 148 - protein, 9 aa, Mus Musculus
WQVTHFPHT

SEQ ID NO: 149 - protein, 10 aa, Mus Musculus
GFSLSTSGMG

SEQ ID NO: 150 - protein, 7 aa, Mus Musculus
IWWDDDK

SEQ ID NO: 151 - protein, 18 aa, Mus Musculus
VRSIYYYDSSPYYYVMDY

SEQ ID NO: 152 - protein, 6 aa, Mus Musculus
QDVSIA

SEQ ID NO: 153 - protein, 3 aa, Mus Musculus
SAS

SEQ ID NO: 154 - protein, 9 aa, Mus Musculus
QQHYSSPLT

SEQ ID NO: 155 - DNA, 340 bp, Mus Musculus
gaggttcagctgcagcagtctggggcagagcttgtgaagccaggggcctcagtcaagttgtcctgcacagcttctggcttcaacattaaagacacccatatgcactgggtgaaacagaggcctgaacagggcctggagtggattggaaagattgatccttcgaatggtaatactcaatatgacccgaagttccagggcaaggccactataacagcagacacatcctccaacacagcctacctgcagctcagcagcctgacatctgaagacactgccgtctattactgtgctacgggatttgcttactggggccaagggactctggtcactgtctctgcag

SEQ ID NO: 156 - DNA, 322 bp, Mus Musculus
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggtatactccgtggacgttcggtggaggcaccaagctggaaatcaagc

SEQ ID NO: 157 - DNA, 361 bp, Mus Musculus
caggttcagctgcagcagtctggggctgagctggtgaggcctgggtcctcagtgaagatttcctgcaaggcttctggctatgcattcagtagctattggatgaactgggtgaagcagaggcctggacagggtcttgagtggattggacagatttatcctggagatggtgatactaactacaatggaaagttcaagggtaaagccacactgactgcagacaaatcctccagcacagcctacatgcagctcagcagcctaacatctgaggactctgcggtctatttatgtgcaagccagatctatgatggttactacacatttacttactggggccaagggactctggtcactgtctctgcag

SEQ ID NO: 158 - DNA, 337 bp, Mus Musculus
gacattgtgatgtcacaatctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggtatctggggtccctgatcgcttcacaggcagtggatctgggacagatttcactctcaccataagcagtgtgcaggctgaagacctggcagtttattactgcaagcaatcttataatctgtggacgttcggtggaggcaccaagctggaaatcaagc

SEQ ID NO: 159 - DNA, 358 bp, Mus Musculus
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacagtggagagccaacatatgttgatgacttcaagggacggtttgccttctctttggaaacctctgccagcactgcctatttgcagatcaacaacctcaaaaatgaggacatggctacatatttctgtgcaagaagcccgggcgcctactatactctggactactggggtcaaggaacctcagtcaccgtctcctcag

SEQ ID NO: 160 - DNA, 337 bp, Mus Musculus
gacattgtgatgtcacagtctccatcctccctggctgtgtcagcaggagagaaggtcactatgagctgcaaatccagtcagagtctgctcaacagcagaacccgaaagaactacttggcttggtaccagcagaaaccagggcagtctcctaaactactgatctactgggcatccactagggaatctggggtccctgatcgcttcacaggcagtggatctgggacagatttcactctcaccatcagcagtgtgcaggctgaagacctggcagtttattactgcaagcaatcttataatctctacacgttcggaggggggaccaagctggaaataaaac

SEQ ID NO: 161 - DNA, 358 bp, Mus Musculus
cagatccagttggtgcagtctggacctgagctgaagaagcctggagagacagtcaagatctcctgcaaggcttctggttataccttcacaaactatggaatgaactgggtgaagcaggctccaggaaagggtttaaagtggatgggctggataaacacctacactggagagccaacatatactgatgacttcaagggacggtttgccttctctttggaaacctctgccagcactgcctatttgcagatcaacaacctcaaaaatgaggacacggctacatatttctgtggaagaggaatacgggattactatactatggactactggggtcaaggaacctcagtcaccgtctcctcag

SEQ ID NO: 162 - DNA, 337 bp, Mus Musculus
gacattgtgatgtcacagtctccatcctccctggctgtgtcagcaggagagaaggtcactatgagctgcaaatccagtcagagtctgctcaacaatagaacccgaaagaactacttggcttggtaccagcagaaaccagggcagtctcctaaactactgatctactgggcatccactagggaatctggggtccctgatcgcttcacaggcagtggatctgggacagatttcactctcaccatcagcagtgtgcaggctgaagacctggcagtttattactgcaaacaatcttataatctttacacgttcggcggggggaccaagctggaaataaaac

SEQ ID NO: 163 - DNA, 361 bp, Mus Musculus
gatgtgcaggttcaggagtcgggacctggcctggtgaaaccttctcagtctctgtccctcacctgcactgtcactggctactcaatcaccagtgattatgcctggacctggatccggcagtttccaggaaacaaactggagtggatgggctacataagctacagtggtagcactagctacaacccatctctcaaaagtcgactgtctatcactcgagacacatccaagaaccagttcttcctgcagttgaattctgtgactactgaggacacagccacatattactgtgcagcctactataggtacggccttgcctactttgcttactggggccaagggactctggtcactgtctctgcag

SEQ ID NO: 164 - DNA, 337 bp, Mus Musculus
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgttggcaaggtacacattttcctcagacgttcggtggaggcaccaagctggaaatcaaac

SEQ ID NO: 165 - DNA, 358 bp, Mus Musculus
cagatccagttggtgcagtctggacctgagctgaagaagcctggagagacagtcaagatctcctgcaaggcttctgggtataccttcacgaactttggaatgaactgggtgaagcaggctccaggaaagggtttaaagtggatgggctggataaacaccttcactggagagccaacatatgttgatgacttcaagggacggtttgccttctctttggaaacctctgccaccactgcctatttgcagatcaacaacctcaaaaatgaggacacggctacatatttctgtgcaagaagtcctgggagggtctatactctggactactggggtcagggaacctcagtcaccgtctcctcag

SEQ ID NO: 166 - DNA, 337 bp, Mus Musculus
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactcgggaatctggggtccctgatcgcttcacaggcagtggatctgggacagatttcactctcaccatcagcagtgtgcaggctgaagacctggcagtttattactgcaagcaatcttataatctttacacgttcggaggggggaccaagctggaaataaaac

SEQ ID NO: 167 - DNA, 343 bp, Mus Musculus
gaggttcagctccagcagtctgggactgtgctggcaaggcctggggcttccgtgaagatgtcctgcaaggcttctggctacaggtttaccagctactggatgtactgggtaaaacagaggcctggacagggtctagagtggattggtgctatttatcctggaaatagtgatactatctacaaccagaggttcaagggcaaggccacactgactgctgtcacatccgccagcactgcctacatggagctcagcagcctggcaaatgaggactctgcggtctatttctgcacacgcccctactttgactcctggggccaaggcaccactctcacagtctcctcag

SEQ ID NO: 168 - DNA, 337 bp, Mus Musculus
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaaac

SEQ ID NO: 169 - DNA, 361 bp, Mus Musculus
gatgtgcagcttcaggagtcgggacctggcctggtgaaaccttctcagtctctgtccctcacctgcactgtcactggcttctcaatcaccagtgattatgcctggaactggatccggcagtttccaggaaacaaactggagtggatgggcttcataaggtacagtggtaatactaggttcaacccatctctcaaaggtcgaggctctatcactcgagacacatccaagaaccagttcttcctgcagttgaattctgtgactactgaggacacagccacttattactgtgcaagcacgttagaagactcttactggtacttcgatgtctggggcgcagggaccacggtcaccgtctcctcag

SEQ ID NO: 170 - DNA, 337 bp, Mus Musculus
gatgttttgatgacccaaactccactctccctgcctgtcagtcttggagatcaagcctccatctcttgcagatctagtcagagcattgtacatactaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaac

SEQ ID NO: 171 - DNA, 337 bp, Mus Musculus
caggtccaactgcagcagtctggggctgaactggtgaagcctggggcttcagtgaagttgtcctgcaaggcttctggctacaccttcaccagctactatatgttctgggtgaagcagaggcctggacagggccttgagtggattggagagattaatcctagcaatggtggttctaacttcaatgagaagttcaagagcaaggccacactgactgtagacaaatcctccagcacagcatacatgcaactcagcagcctgacatctgaggactctgcggtctattactgtacaagaggggctttctggggccaagggactctggtcactgtctctgcag

SEQ ID NO: 172 - DNA, 337 bp, Mus Musculus
gatgttgtgatgacccagactccactcactttgtcggttaccattggacaaccagcctccatctcttgcaagtcaagtcagagcctcttagatagtgatagaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaagttacacattttccgcacacgttcggtgctgggaccaagctggagctgaaac

SEQ ID NO: 173 - DNA, 379 bp, Mus Musculus
caagttactctaaaagagtctggccctgggatattgaagccctcacagaccctcagtctgacttgttctttctctgggttttcactgagcacttctggtatgggtgtaggctggattcgtcagccttcagggaagggtctggagtggctggcacacatttggtgggatgatgataagtactataacccatccctgaagaaccgcctcacaatctccaaggatacctccagaaaccaggtattcctcaagatcaccagtgtggacactgcagatactgccacttactactgtgttcgaagtatttattactacgatagtagcccttattactatgttatggactactggggtcaaggaacctcagtcaccgtctcctcag

SEQ ID NO: 174 - DNA, 322 bp, Mus Musculus
gacattgtgatgacccagtctcacaaattcatgtccacatcagtaggagacagggtcagcatcacctgcaaggccagtcaggatgtgagtattgctgtagcctggtatcaacagaaaccaggacaatctcctaaactactgatttactcggcatcctaccggaacactggagtccctgatcgcttcactggcagtggatctgggacggatttcactttcaccatcagcagtgtgcaggctgaagacctggcagtttattactgtcagcaacattatagtagtccgctcacgttcggtgctgggaccaagctggagctgaaac

SEQ ID NO: 175 - protein, 20 aa, Oryctolagus cuniculus
METGLRWLLLVAVLKGVQCQ

SEQ ID NO: 176 - protein, 19 aa, Homo Sapiens
MELGLSWIFLLAILKGVQC

SEQ ID NO: 177 - protein, 19 aa, Homo Sapiens
MELGLRWVFLVAILEGVQC

SEQ ID NO: 178 - protein, 19 aa, Homo Sapiens
MKHLWFFLLLVAAPRWVLS

SEQ ID NO: 179 - protein, 19 aa, Homo Sapiens
MDWTWRILFLVAAATGAHS

SEQ ID NO: 180 - protein, 19 aa, Homo Sapiens
MDWTWRFLFVVAAATGVQS

SEQ ID NO: 181 - protein, 19 aa, Homo Sapiens
MEFGLSWLFLVAILKGVQC

SEQ ID NO: 182 - protein, 19 aa, Homo Sapiens
MEFGLSWVFLVALFRGVQC

SEQ ID NO: 183 - protein, 26 aa, Homo Sapiens
MDLLHKNMKHLWFFLLLVAAPRWVLS

SEQ ID NO: 184 - protein, 22 aa, Homo Sapiens
MDMRVPAQLLGLLLLWLSGARC

SEQ ID NO: 185 - protein, 22 aa, Mycobacterium tuberculosis H37Rv
MKYLLPTAAAGLLLLAAQPAMA

SEQ ID NO: 186 - protein, 4 aa, Artificial Sequence
  Synthetic Construct
GGCG

(19) United States
(12) Patent Application Publication
(10) Pub. No.: US 2022/0218805 A1
(43) Pub. Date: Jul. 14, 2022

(54) CONFORMATION-SPECIFIC EPITOPES IN TAU, ANTIBODIES THERETO AND METHODS RELATED THEREOF

(71) Applicants: THE UNIVERSITY OF BRITISH COLUMBIA, Vancouver (CA); PROMIS NEUROSCIENCES INC., Toronto (CA)

(72) Inventors: Steven S. Plotkin, Vancouver (CA); Neil R. Cashman, Vancouver (CA); Johanne Kaplan, Sherborn, MA (US)

(21) Appl. No.: 17/614,487

(22) PCT Filed: May 27, 2020
(86) PCT No.: PCT/CA2020/050722

§ 371 Date: Nov. 26, 2021

Related U.S. Application Data
(60) Provisional application No. 62/853,121, filed on May 27, 2019.
(60) Provisional application No. 62/915,931, filed on Oct. 16, 2019.

(51) Int. Cl. A61K 39/00; A61K 39/39; C07K 16/18; A61P 25/28; C12N 5/16

(52) U.S. Cl. CPC A61K 39/0005; A61K 39/39; C07K 16/18; C07K 2317/56; C12N 5/163; A61K 2039/55505; C07K 2317/565; A61P 25/28

(57) ABSTRACT

The disclosure pertains to conformational epitopes in oligomeric tau, antibodies thereto and methods of making and using immunogens and antibodies specific thereto. The antibodies bind activity neutralizing sites in tau. Also provided are methods for making and using, including methods for treating a tauopathy.

### RELATED APPLICATIONS

This Patent Cooperation Treaty application claims the benefit of priority of U.S. Provisional Application 62/853,121 filed May 27, 2019 and U.S. Provisional Application, 62/915,931 filed Oct. 16, 2019, each of which are incorporated herein in their entirely.

### FIELD

The present disclosure relates to tau epitopes and antibodies thereto, and more specifically to sequence and conformationally specific tau epitopes that are predicted to be selectively accessible in misfolded (e.g. oligomeric) tau, and related antibody compositions, methods of making and uses thereof.

### BACKGROUND

The tau protein plays a key role in stabilizing the microtubules in central nervous system neurons. The development of misfolded forms of tau leads to toxicity and abnormal microtubule function seen in Alzheimer's disease (AD), frontotemporal dementia and other tauopathies such as those due to repetitive head injury (chronic traumatic encephalopathy).

Antibodies to oligomeric tau have been described, for example, in U.S. Pat. No. 8,778,343.

Several therapeutic tau antibodies are in development but it remains unclear whether they are directed against the most effective target epitopes and tau species. For example, antibodies binding N-terminal epitopes have been reported to be poor inhibitors of tau seeding and aggregation (Courade et al, 2018, Acta Neuropathologica). Two antibodies directed against N-terminal epitopes, from Biogen and Abbvie, have failed in progressive supranuclear palsy (PSP) clinical trials. Binding of antibodies to monomers can result in the “soaking up” of therapeutic antibodies by physiological tau.

Antibodies that preferentially or selectively bind misfolded oligomeric tau over monomeric tau and inhibit or neutralize tau seeding or other pathological activity are desirable.

### SUMMARY

Described herein are conformational epitopes in tau. The conformational epitopes are sequence and conformationally specific epitopes where antibodies recognize a particular amino acid sequence that is conformationally distinct in misfolded oligomeric tau polypeptide and/or soluble fibrils compared to monomeric tau polypeptide. The inventors have determined that several residues are selectively exposed in misfolded oligomeric tau polypeptide and have designed reagents and produced antibodies that are selective for misfolded oligomeric tau polypeptide and/or soluble fibrils at activity neutralizing sites. Other aspects described herein include methods of making said reagents and antibodies and methods of using thereof for detecting misfolded oligomeric tau. The epitopes are selectively exposed in misfolded oligomeric species of tau, and less available in tau monomer.

An aspect includes a cyclic compound comprising a tau peptide comprising and/or consisting of 4 or more residues of KLDFK (SEQ ID NO: 1), optionally KLDF (SEQ ID NO: 2), LDFK (SEQ ID NO: 3), or KLDFK (SEQ ID NO: 1) and optionally a linker. Also provided is a linear compound comprising a tau peptide comprising and/or consisting of 4 or more residues of KLDFK (SEQ ID NO: 1), optionally KLDF (SEQ ID NO: 2), LDFK (SEQ ID NO: 3), or KLDFK (SEQ ID NO: 1), and optionally a linker.

A further aspect is an immunogen comprising a cyclic compound described herein. Immunogens are immunogenic.

Another aspect includes an antibody that selectively binds the tau peptide in the cyclic compound described herein and/or soluble fibrils compared to a corresponding linear compound and/or binds misfolded oligomeric tau selectively compared to monomeric tau polypeptide and/or raised using an immunogen or composition comprising said immunogen described herein. Such antibodies may bind a different epitope and/or have improved binding characteristics and/or targeting characteristics over existing antibodies that bind tau. For example, the antibodies were raised to an immunogen that mimics a conformational epitope at a tau activity neutralizing site.

Also included is a nucleic acid encoding an antibody described herein. In some embodiments, the nucleic acid is comprised in a vector.

A further aspect includes a method of reducing or inhibiting tau aggregation/aggregates and/or propagation, comprising contacting a cell or tissue expressing misfolded oligomeric tau polypeptide and/or soluble fibrils, with an antibody herein disclosed.

Another aspect herein disclosed relates to a method of treating a tauopathy in a subject in need thereof, comprising administering to the subject an effective amount of an antibody herein disclosed or a composition comprising said antibody.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1A is a schematic representation of tau comprising 10 chains as shown in PDB 5O3L. FIG. 1B is a schematic representation of tau comprising 10 chains after collective coordinate biasing to partially disorder the fibril structure.

FIG. 2A is a schematic illustrating the prediction strength of the predicted epitopes and FIG. 2B is a schematic representation of tau PDB 5O3L with the predicted KLDFK (SEQ ID NO: 1) epitope superimposed.

FIGS. 3A, 3B and 3C are scatter plots of the Jensen-Shannon distance (JSD), a measure of the dissimilarity between two ensembles. X-axis is the JSD between cyclic peptide and tau monomer, Y-axis is the JSD between cyclic peptide and biased or stressed (or biased) fibril. Each point in the plot corresponds to a given cyclic peptide scaffold.

FIG. 4 is a scatter plot illustrating binding of raised antibodies (hybridoma supernatants) to immobilized tau oligomers.

FIG. 5 is a scatter plot illustrating binding of raised antibodies (hybridoma supernatants) to immobilized tau monomers.

FIG. 6 is a bar graph superimposing results of binding to scatter plot illustrating binding of antibodies (hybridoma supernatants) to tau oligomers and tau monomers for each antibody raised.

FIG. 7 is a scatter plot that shows the ratio of the binding response to tau oligomers/tau monomers for each antibody (hybridoma supernatants).

FIGS. 8 A-H are a series of graphs that shows the binding of purified antibodies to different concentrations of tau oligomers or monomers in SPR assays. FIGS. 8I and 8J are graphs that show controls.

FIG. 9 is a bar graph illustrating binding of purified mAb clone 8G7 (test mAb) with tau species in soluble brain extract from AD patients in SPR assays.

FIG. 10 is a series of bar graphs illustrating binding of purified mAbs to tau species in soluble brain extract from a single AD brain (left panel) or a pool of three AD brains (right panel) in SPR assays.

FIG. 11 is a bar graph illustrating the binding of purified antibodies to soluble tau pre-formed fibrils (PFF) in SPR assays.

FIG. 12 is a bar graph illustrating the ability of purified antibodies to inhibit or reduce tau pre-formed tau fibrils (PFF)-induced formation of intracellular tau aggregates using a cellular Fluorescence Energy Resonance Transfer (FRET) assay.

FIG. 13 is a bar graph showing the ability of tau mAbs to inhibit seeding activity of AD brain homogenate as assessed in a FRET assay using Tau RD P301S FRET Biosensor cells.

FIG. 14 is a bar graph showing the ability of tau mAbs to bind and deplete AD brain seeds.

### DETAILED DESCRIPTION OF THE DISCLOSURE

Demonstrated herein is the generation of conformation-specific antibodies and immunogens for generating said antibodies. The inventors have identified a target more likely to be present on misfolded oligomeric tau polypeptide at an activity neutralizing site.

Antibodies raised to native protein regions tend not to be selective for misfolded protein such as oligomeric species, and thus may bind to native tau protein as well as misfolded protein.

As described herein, to develop antibodies selective for misfolded oligomeric forms of tau, the inventors identified a region of tau sequence that may be prone to disruption in the context of the fibril, and that may thus be exposed on the surface of the misfolded protein oligomers where they may act as catalytic substrates for misfolding. The region of tau identified may be important for misfolded tau disease activity as antibodies that bind said conformation are able to inhibit misfolded tau seeding and misfolded tau propagation.

An experimentally-validated structural model of the fibril structure was globally biased away from its reported conformation to be partially unfolded, using molecular dynamics, to yield regions of contiguous primary sequence that are prone to be disordered upon an external challenge such as an anomalous cellular environment.

It was hypothesized that these weakly-stable regions may be selectively exposed in misfolded oligomeric proteins, or misfolded pathogenic species. They may thus constitute oligomer-selective epitope predictions and/or predictions that differentiate from native monomeric tau.

As described the Examples, the inventors designed cyclic compounds comprising the identified epitopes to mimic the putative selective epitope by satisfying several criteria. Monoclonal antibodies were produced using immunogens comprising the cyclic compounds described herein to produce antibodies that preferentially bind oligomeric tau and which are able to inhibit misfolded tau seeding and misfolded tau propagation.

### I. Definitions

As used herein, the term “tau” as used herein and depending on the context can mean all forms and isoforms of tau including wildtype sequence tau, monomeric tau, as well as misfolded forms including mutant forms thereof from all species, particularly human tau (i.e. hutau). In human brain, tau proteins constitute a family of alternatively spliced isoforms with a range of 352-441 amino acids. The longest isoform in the central nervous system (tau-F or tau-4) has four repeat units (R1, R2, R3 and R4) and two inserts, with 441 amino acids total, while the shortest isoform has three repeats (R1, R3 and R4) and no insert, with 352 amino acids total. The amino acid sequence (e.g. Uniprot Accession number for tau-4, P10636-8) and the nucleotide sequence (e.g. NCBI Gene name/ID: MAPT/4137) have been previously characterized.

“Wild type” as used herein refers to the primary amino acid sequence of any isoform of non-mutant or naturally occurring tau protein, for example as found, in humans.

“Native tau polypeptide” as used herein refers to the tau monomer whether associated with microtubules or cytosolic found in normal cells. Isolated monomeric structures can be predicted using one of the chains from the PDB fibril (PDB 5O3L) as described herein. Native tau polypeptide can be detected using pan antibodies in for example brains not afflicted by a tauopathy.

The term “tauopathy” as used herein refers to a class of neurodegenerative diseases associated with pathological aggregation of tau protein and include for example, Alzheimer's disease (AD), Pick's disease, frontotemporal dementia or frontotemporal lobar degeneration, progressive supranuclear palsy, corticobasal degeneration, primary age-related tauopathy, chronic traumatic encephalopathy, subacute sclerosing panencephalitis, frontotemporal dementia and parkinsonism linked to chromosome 17.

“Structured fibril”, “un-stressed fibril”, or “unbiased fibril” as used herein refers to the expected conformations that would be observed in thermal equilibrium for a fibril of tau protein, e.g. for which PDB 5O3L would be a representative example of.

“Misfolded oligomer” as used herein refers to the secondary and tertiary structure of a multisubunit polypeptide or polypeptide aggregation, and indicates that the oligomeric polypeptide, or a subunit therein has adopted a conformation (e.g. at one or more locations) that is different from that typically adopted by the native monomer. Although misfolding can be caused by mutations in a protein, such as amino acid deletion, substitution, or addition, wild-type sequence protein can also be misfolded in disease, and expose disease-specific or disease-selective epitopes for instance, as a result of a change in microenvironmental conditions, or oligomer formation that may be on- or off-pathway to fibril formation. Accordingly, “misfolded oligomeric tau polypeptide”, or “misfolded oligomeric tau” when referring to the polypeptide herein refers to tau polypeptide oligomers wherein the subunits thereof display a conformation that is different from a unit of monomeric tau. For example, misfolded oligomeric tau can include a conformation that is partially-ordered, containing parts of the fibril structure, and partially-disordered, containing polymer segments of amino acids that have alternate conformations than either monomer, and/or fibril tau. Misfolded oligomeric tau includes conformational epitopes that are selectively presented or accessible for binding wherein the epitope sequence in misfolded oligomeric tau can be conformationally different than the corresponding sequence in the context of the monomer.

The term “soluble fibril” as used herein refers to fibril fragments and protofibrils that are soluble in interstitial fluid.

The term “mutant tau” refers to forms of tau, and particularly endogenous forms of tau that occur as a result of genetic mutation that result for instance in amino acid substitution, such as those substitutions characteristic for instance of frontotemporal dementia (FTD). tau protein mutations are generally not linked to familial forms of AD, but can cause FTD and several other tauopathies (including those involved in Pick's disease, Progressive Supranuclear Palsy, and Parkinson's disease; see e.g. https://www.alzforum.org/mutations/mapt for a list of known pathogenic mutations, incorporated herein by reference).

The term “KLDF (SEQ ID NO: 2)” means the amino acid sequence: lysine, leucine, asparagine, phenylalamine as shown in SEQ ID NO: 2. Similarly KLDFK, (SEQ ID NO: 1), and other sequences refer to the amino acid sequences identified by the 1-letter amino acid code. Depending on the context, the reference of the amino acid sequence can refer to a sequence in tau or an isolated peptide, such as the amino acid sequence of the epitope portion of a cyclic compound. The sequences KLDF (SEQ ID NO: 2) and LDFK (SEQ ID NO: 3) consist of residues 343-346 and residues 344-347 in the tau amino acid primary sequence as shown in P10636-8, respectively. As mentioned there are other isoforms of tau and a person of skill in the art would readily be able to confirm the numbering in another isoform. For example, KLDFK (SEQ ID NO: 1) is amino acids 283-287 in isoform tau-b corresponding to fasta file P10636-4.

The amino acid sequence KLDFK (SEQ ID NO: 1) is present in all 6 tau isoforms expressed in human brain.

The term “an epitope in KLDFK (SEQ ID NO: 1)” as used herein refers to any part thereof that is specifically bound by an antibody.

The term “epitope” as used herein means a sequence of amino acids in an antigen wherein the amino acids (or a subset thereof) in the sequence are specifically recognized by an antibody or binding fragment, for example an antibody or binding fragment described herein. An epitope can comprise one or more antigenic determinants. For example, an antibody generated against an isolated peptide corresponding to a conformational epitope recognizes part or all of said epitope sequence.

The term “epitope selectively presented or accessible in misfolded oligomeric tau” as used herein refers to a conformational epitope that is selectively presented or accessible on misfolded oligomeric tau as present in tauopathies such as AD and FTD whether in multimeric, oligomeric, or aggregated forms, but not on the molecular surface of either the monomeric polypeptide of tau or on the surface of microtubule bound tau, as found normally in vivo.

As used herein, the term “conformational epitope” refers to a sequence of amino acids or an antigenic determinant thereof that has a particular three-dimensional structure in a species of a protein wherein at least an aspect of the three-dimensional structure is present or is more accessible to antibody binding compared to in another species such as a corresponding unbiased fibril structure or a monomer structure, or microtubule-associated tau protein. Antibodies which selectively bind a conformational epitope relative to another conformation, recognize the spatial arrangement of one or more of the amino acids of that conformation-specific epitope. For example, a conformational epitope in KLDFK (SEQ ID NO: 1) can refer to a conformation of KLDFK (SEQ ID NO: 1) that is recognized by antibodies selectively, for example at least 1.5 fold, at least 2 fold, at least 2.5 fold, at least 3 fold, at least 3.5 fold or at least 4 fold or greater more selectivity as compared to another conformation, optionally the region in the tau monomer or for example antibodies raised using a corresponding linear peptide or part thereof.

Reference to the “cyclic peptide” herein can refer to a fully proteinaceous compound (e.g. wherein the linker is 2, 3, 4, 5, 6, 7 or 8 amino acids). It is understood that properties described for the cyclic peptide determined in the examples can be incorporated in other compounds (e.g. cyclic compounds) comprising non-amino acid linker molecules. “Cyclic peptide” and “cyclic compound” can be used interchangeably when the cyclic compound is composed of amino acids.

The term “amino acid” includes all of the naturally occurring amino acids as well as modified L-amino acids. The atoms of the amino acid can for example include different isotopes. For example, the amino acids can comprise deuterium substituted for hydrogen, nitrogen-15 substituted for nitrogen-14, and carbon-13 substituted for carbon-12 and other similar changes.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-group size for one another. Examples of conservative amino acid substitution include:

Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

The term “antibody” as used herein is intended to include monoclonal antibodies, polyclonal antibodies, single chain, humanized and other chimeric antibodies as well as binding fragments thereof. The antibody may be from recombinant sources and/or produced in transgenic animals. Also included are human antibodies that can be produced through using biochemical techniques or isolated from a library. Humanized or chimeric antibody may include sequences from one or more than one isotype or class. Reference to antibody or antibodies of the disclosure refers to an antibody or antibodies described herein that are for example raised to an immunogen described herein and/or selective for an epitope described herein for example LDFK (SEQ ID NO: 3), KLDF (SEQ ID NO: 2) or KLDFK (SEQ ID NO: 1) or a part thereof in the context for example of the epitope, misfolded oligomeric tau, and/or a conformational compound comprising one of said epitopes sequences.

The phrase “isolated antibody” refers to antibody produced in vivo or in vitro that has been removed from the source that produced the antibody, for example, an animal, hybridoma or other cell line (such as recombinant cells that produce antibody). The isolated antibody is optionally “purified”, which means at least: 80%, 85%, 90%, 95%, 98% or 99% purity.

The term “complementarity determining region” or “CDR” as used herein refers to particular hypervariable regions of antibodies that are commonly presumed to contribute to epitope binding. Computational methods for identifying CDR sequences include Kabat, Chothia, and IMGT. The CDRs listed in the present disclosure are identified using IMGT Blast. A person skilled in the art having regard to the sequences comprised herein would also be able to identify CDR sequences based on Kabat and Chothia etc. Such antibodies are similarly encompassed.

The term “binding fragment” as used herein to a part or portion of an antibody or antibody chain comprising fewer amino acid residues than an intact or complete antibody or antibody chain and which binds the antigen or competes with intact antibody. Exemplary binding fragments include without limitations Fab, Fab′, F(ab′)2, scFv, dsFv, ds-scFv, dimers, nanobodies, minibodies, diabodies, and multimers thereof. Fragments can be obtained via chemical or enzymatic treatment of an intact or complete antibody or antibody chain. Fragments can also be obtained by recombinant means. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be constructed by recombinant expression techniques.

When an antibody is said to bind to an epitope, such as KLDFK (SEQ ID NO:1), what is meant is that the antibody specifically binds to a polypeptide or compound containing the specified residues or a part thereof for example at least 1 residue or at least 2 residues. Such an antibody does not necessarily contact every residue of KLDFK (SEQ ID NO: 1), and every single amino acid substitution or deletion within said epitope does not necessarily significantly affect or equally affect binding affinity.

The term “detectable label” as used herein refers to moieties such as peptide sequences, fluorescent proteins that can be appended or introduced into a peptide or compound described herein and which is capable of producing, either directly or indirectly, a detectable signal. For example, the label may be radio-opaque, positron-emitting radionuclide (for example for use in PET imaging), or a radioisotope, such as ³H, ¹³N, ¹⁴C, ¹³F, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion. The detectable label may be also detectable indirectly for example using secondary antibody.

The term “greater affinity” as used herein refers to a degree of antibody binding where an antibody X binds to target Y more strongly (Kₒₙ) and/or with a smaller dissociation constant (K_{off}) than to target Z, and in this context antibody X has a greater affinity for target Y than for Z. Likewise, the term “lesser affinity” herein refers to a degree of antibody binding where an antibody X binds to target Y less strongly and/or with a larger dissociation constant than to target Z, and in this context antibody X has a lesser affinity for target Y than for Z. The affinity of binding between an antibody and its target antigen, can be expressed as K_{A} equal to 1/K_{D} where K_{D} is equal to k_{off}/kₒₙ. The kₒₙ and k_{off} values can be measured using surface plasmon resonance (measurable for example using a Biacore system).

Also, as used herein, the term “immunogenic” refers to substances which elicit the production of antibodies, activate lymphocytes or other reactive immune cells directed against an antigenic portion of the immunogen.

An “immunogen” as used herein means a substance which provokes an immune response and causes production of an antibody and can comprise for example cyclic peptides described herein, conjugated as multiantigenic peptide and/or fused to an immunogenicity enhancing agent such as Keyhole Limpet Hemocyanin (KLH). In addition to the conjugates described herein, immunogenic peptide mimetics which elicit cross-reactive antibodies to the epitopes identified, e.g. KLDFK (SEQ ID NO: 1), KLDF (SEQ ID NO: 2) or LDFK (SEQ ID NO: 3) constitute immunogens. To serve as a useful immunogen, the tau peptide desirably incorporates a minimum of about, 4, 5, 6, or 7 tau residues, comprising for example 4 or more of K343, L344, D345, F346, K347, and optionally 1, 2 or 3 additional flanking residues in tau, for example up to two residues N-terminus and up to 3 residues C-terminus in the context of a cyclic compound. The immunogen can also be larger, for example up to 12 or 13 amino acids or subunits and comprising a tau peptide, for example KLDF (SEQ ID NO: 2) or LDFK (SEQ ID NO: 3).

The term “corresponding linear compound” with regard to a cyclic compound refers to a compound, optionally a peptide, comprising or consisting of the same sequence or chemical moieties as the cyclic compound but in linear (non-cyclized) form.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense. Further, the term “nucleic acid” includes the complementary nucleic acid sequences as well as codon optimized or synonymous codon equivalents. The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived.

The term “selective” or “selectively binds” as used herein with respect to an antibody that preferentially binds a form of tau (e.g. monomer, or misfolded oligomeric protein) means that the binding protein binds the form with at least 1.5 fold, 2 fold, at least 3 fold, at least 3.5 fold, at least 4 fold, at least 5 fold, or more greater affinity. Accordingly, an antibody that is more selective for a particular conformation (e.g. misfolded protein) preferentially binds the particular form of tau with at least 2 fold etc. greater affinity compared to another form.

The term “linker” as used herein means a chemical moiety, preferably poorly immunogenic or non-immunogenic, that can be covalently linked directly or indirectly to the tau peptide N- and/or C-termini comprising at least 3 amino acids of KLDFK (SEQ ID NO:1), optionally KLDF (SEQ ID NO: 2), or LDFK (SEQ ID NO: 3) epitope peptide, which is linked to the peptide N- and/or C-termini. The linker ends can for example be joined to produce a cyclic compound. The linker can comprise one or more functionalizable moieties such as one or more cysteine (C) residues. The linker can be linked via the functionalizable moieties to a carrier protein or an immunogen enhancing agent such as keyhole limpet hemocyanin (KLH) or bovine serum albumin (BSA). The cyclic compound comprising the linker is of longer length than the peptide itself. That is, when cyclized the peptide with a linker (for example of 3 amino acid residues) makes a larger closed circle than the peptide without a linker. The linker may include, but is not limited to, non-immunogenic moieties such as amino acids Glycine (G), and Alanine (A), or polyethylene glycol (PEG) repeats. The linker can be for example 9 amino acids, optionally GGGGCGGGG (SEQ ID NO: 74), or 8 amino acids, optionally GGGCGGGG (SEQ ID NO: 67), GGCGGGGG (SEQ ID NO: 68) or GCGGGGGG (SEQ ID NO: 69) or 7 amino acids, optionally GGGGCGG (SEQ ID NO: 65), GGGCGGG (SEQ ID NO: 70), GGCGGGG (SEQ ID NO: 71) or GCGGGGG (SEQ ID NO: 72), 6 amino acids, optionally GGGCGG (SEQ ID NO: 73), GGCGGG (SEQ ID NO: 45) or GCGGGG (SEQ ID NO: 47), 5 amino acids optionally, GCGGG (SEQ ID NO: 44) or GGGCG (SEQ ID NO: 46), 4 amino acids such as GCGG (SEQ ID NO: 43) or GGCG (SEQ ID NO: 186) or 3 amino acids such as GCG. Linkers can be referred to according to the number of residues on either end of a peptide for example 3,1 refers to a linker that has a functionalizable moiety such as cysteine and 3 amino acids, that are N terminal and 1 amino acid that is C terminal the tau peptide. Examples of linkers are provided in SEQ ID NOs: 186, 43-47 and 65-74.

The term “functionalizable moiety” as used herein refers to a chemical entity with a “functional group” which as used herein refers to a group of atoms or a single atom that will react with another group of atoms or a single atom (so called “complementary functional group”) to form a chemical interaction between the two groups or atoms. In the case of cysteine (C), the functional group can be —SH which can be reacted to form a disulfide bond. Accordingly, the linker can for example be CCC. The reaction with another group of atoms can be covalent or a strong non-covalent bond, for example as in the case as biotin-streptavidin bonds, which can have Kd≈1e-14. A strong non-covalent bond as used herein means an interaction with a Kd of at least 1e-9, at least 1e-10, at least 1e-11, at least 1e-12, at least 1e-13 or at least 1e-14.

Proteins and/or other agents may be coupled to the cyclic compound, either to aid in immunogenicity, or to act as a probe in in vitro studies. For this purpose, any functionalizable moiety capable of reacting (e.g. making a covalent or non-covalent but strong bond) may be used. In one specific embodiment, the functionalizable moiety is a cysteine residue which is reacted to form a disulfide bond with an unpaired cysteine on a protein of interest, which can be, for example, an immunogenicity enhancing agent such as Keyhole limpet hemocyanin (KLH), or a carrier protein such as Bovine serum albumin (BSA) used for in vitro immunoblots or immunohistochemical assays.

The term “reacts with” as used herein generally means that there is a flow of electrons or a transfer of electrostatic charge resulting in the formation of a chemical interaction.

The term “animal” or “subject” as used herein includes all members of the animal kingdom including mammals, optionally including or excluding humans.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a presymptomatic subject can be treated to prevent progression. Such a subject can be treated with a compound, antibody, immunogen, immunoconjugate or composition described herein to prevent progression.

As used herein, the phrase “effective amount” means an amount effective, at dosages and for periods of time necessary to achieve a desired result. Effective amounts when administered to a subject may vary according to factors such as the disease state, age, sex, weight of the subject. Dosage regime may be adjusted to provide the optimum therapeutic response.

Compositions or methods “comprising” or “including” one or more recited elements may include other elements not specifically recited. For example, a composition that “comprises” or “includes” an antibody may contain the antibody alone or in combination with other ingredients.

The term “administered” as used herein means administration of a therapeutically effective dose of a compound or composition of the disclosure to a cell or subject.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g. 1 to 5 includes 1, 1.5, 2, 2.75, 3, 3.90, 4, and 5 and the like). It is also to be understood that all numbers and fractions thereof are presumed to be modified by the term “about.”

Further, terms of degree such as “substantially”, “about” and “approximately” as used herein mean a reasonable amount of deviation of the modified term such that the end result is not significantly changed.

The term “about” means plus or minus 0.5%. 1%, 2%, 5%, 10%, 15%, or 20% of the number to which reference is being made.

Further, the definitions and embodiments described in particular sections are intended to be applicable to other embodiments herein described for which they are suitable as would be understood by a person skilled in the art. For example, in the following passages, different aspects are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The singular forms of the articles “a,” “an,” and “the” include plural references unless the context clearly dictates otherwise. For example, the term “a compound” or “at least one compound” can include a plurality of compounds, including mixtures thereof.

### II. Epitopes

The inventors have identified epitopes in tau protein including KLDFK (SEQ ID NO: 1), KLDF (SEQ ID NO: 2), and LDFK (SEQ ID NO: 3) at amino acid positions 343-347, 343-346, and 344-347 respectively (Indexed according to Fasta file P10636-8.fasta of isoform tau-F). They have further identified that the epitopes or parts thereof may be conformational epitopes, and that KLDF (SEQ ID NO: 2) and LDFK (SEQ ID NO: 3) or a part of either of thereof may be selectively accessible to antibody binding in misfolded oligomeric species of tau.

Based on one or more conformational differences identified between the epitopes identified in monomeric, and biased tau fibril ensembles, the inventors have designed conformationally restricted compounds and immunogens for producing antibodies.

As shown in the Examples, antibodies raised using said immunogens are useful for detecting or targeting misfolded oligomeric tau.

As described in the Examples, cyclic compounds such as cyclic peptides described in Tables 2 and 4 and the cyclic constructs used to raise antibodies, e.g. CGGGKLDFG (SEQ ID NO: 15 (3,1 linker)), CGGGKLDFGG (SEQ ID NO:16 (3,2 linker)) and CGGGGKLDFG (SEQ ID NO:19 (4,1 linker)), were determined to capture conformational differences of the corresponding epitope in misfolded oligomeric species of tau relative to monomeric species. This suggests that the cyclic compounds may provide for a conformational epitope that is conformationally-distinct from the sequence as presented in the monomeric tau.

Accordingly, the present disclosure identifies conformational epitopes in tau consisting of amino acids KLDF (SEQ ID NO: 2), LDFK (SEQ ID NO: 3), or KLDFK (SEQ ID NO: 1) or a part thereof such as FK corresponding to amino acids residues 346-347 on tau or DFK corresponding to amino acids 345-347 on tau. As demonstrated in the Examples, KLDFK (SEQ ID NO: 1) or parts thereof such as KLDF (SEQ ID NO: 2) or LDFK (SEQ ID NO: 3) were identified as regions prone to disorder in stressed tau fibrils. The residues KLDF (SEQ ID NO: 2) and LDFK (SEQ ID NO: 3), emerged in a prediction using the Collective Coordinates method as described in the Examples.

An aspect includes a compound comprising a tau peptide comprising at least at least 3 or at least 4 amino acids of KLDFK (SEQ ID NO: 1), optionally KLDF (SEQ ID NO: 2), LDFK (SEQ ID NO: 3) or KLDFK (SEQ ID NO: 1). In an embodiment, the tau peptide is selected from KLDF (SEQ ID NO: 2), or LDFK (SEQ ID NO: 3).

The tau peptide can also include 1, 2 or 3 amino acids in tau either N-terminal and/or C-terminal to KLDFK (SEQ ID NO: 1) or an internal sequence there of such KLDF (SEQ ID NO: 2) with 1, 2 or 3 N-terminal amino acid residues, or LDFK (SEQ ID NO: 3) with 1, 2 or 3 C-terminal amino acid residues. In one embodiment, the tau peptide comprises up to 2 amino acids N-terminal and/or up to 3 amino acids C terminal of SEQ ID NO: 1.

In an embodiment, the compound further includes a linker. The linker can comprise one or more functionalizable moieties. The linker can for example comprise 1, 2, 3, 4, 5, 6, 7, 8 or 9 amino acids and/or equivalently functioning molecules such as polyethylene glycol (PEG) moieties, and/or a combination thereof. In an embodiment, the linker amino acids are selected from non-immunogenic or poorly immunogenic amino acid residues such as G, or A, for example the linker can be GG, GGG, GAG, G(PEG)G, PEG-PEG(also referred to as PEG2)-GG and the like. One or more functionalizable moieties e.g. amino acids with a functional group may be included for example for coupling the compound to an agent or detectable tag or a carrier such as BSA or an immunogenicity enhancing agent such as KLH.

In an embodiment, the linker comprises 1, 2, 3, 4, 5, 6, 7, 8 or 9 amino acids.

In an embodiment, the linker comprises GC-PEG, PEG-GC, GCG or PEG2-CG. In another embodiment, the linker comprises or consists of GGCG (SEQ ID NO: 186; 1,2 linker), GCGG (SEQ ID NO: 43; 2,1 linker), GCG (1,1 linker), GCGGG (SEQ ID NO:44; 3,1 linker), GGCGGG (SEQ ID NO: 45; linker 3, 2), GGGCG (SEQ ID NO: 46; 1,3 linker), or GCGGGG (SEQ ID NO: 47; linker 4, 1).

In an embodiment, the linker comprises or consists of GGCG (SEQ ID NO: 186; 1,2 linker). In an embodiment, the linker comprises or consists of GCGG (SEQ ID NO: 43; 2,1 linker). In an embodiment, the linker comprises or consists of GCG (1,1 linker). In an embodiment, the linker comprises or consists of GCGGG (SEQ ID NO: 44; 3,1 linker). In an embodiment, the linker comprises or consists of GGCGGG (SEQ ID NO: 45; linker 3,2). In an embodiment, the linker comprises or consists of GGGCG (SEQ ID NO: 46; 1,3 linker). In an embodiment, the linker comprises or consists of GCGGGG (SEQ ID NO: 47; linker 4,1). Other linkers are provided (presented in constructs comprising the tau peptide) in Tables 2, 4 and/or 7. In an embodiment, the linker comprises or consists of a sequence selected from any one of SEQ ID NOs: 65-74.

Proteinaceous portions of compounds (or the compound wherein the linker is also proteinaceous) may be prepared by chemical synthesis using techniques well known in the chemistry of proteins such as solid phase synthesis or synthesis in homogenous solution.

The compound can be linear. Preferably, the compound is a conformational compound such that at least the one of the K343, the L344, the D345, the F346, and/or K347 residues is in an alternate conformation in the compound than the corresponding residues in a monomeric and/or fibril ensemble. As shown in the Examples this can be accomplished using a cyclic peptide comprising the tau peptide.

An aspect therefore provides a compound, optionally a cyclic compound, comprising a tau peptide comprising at least 4 amino acids of KLDFK (SEQ ID NO: 1), optionally KLDF (SEQ ID NO: 2), or LDFK (SEQ ID NO: 3), and a linker, wherein the linker is covalently coupled directly or indirectly to the tau peptide. In an embodiment, the compound is a cyclic compound. In an embodiment, the cyclic compound comprises a tau peptide and linker described herein. In an embodiment, the cyclic compound comprises a tau peptide comprising KLDF (SEQ ID NO: 2), or LDFK (SEQ ID NO: 3), and up to 6 tau residues (e.g. 1 or 2 or 3 amino acids N and/or C terminus to KLDF (SEQ ID NO: 2) or LDFK (SEQ ID NO: 3), and a linker, wherein the linker is covalently coupled directly or indirectly to the peptide N-terminus residue and the C-terminus residue of the tau peptide. The exposure of the residues in the cyclic peptide can be different than corresponding residues, in the monomeric and/or fibril ensembles or cellular monomeric and/or fibrillary tau. For example, in the cyclic compound, at least one of K343, L344, D345, F346 and/or K347 has more surface exposure than the conformation occupied in the fibril ensemble.

In embodiments wherein the peptide comprising KLDF (SEQ ID NO: 2), includes 1, 2 or 3 additional residues found in tau that are N- and/or C-terminal to KLDF (SEQ ID NO: 2) the linker in the cyclized compound is covalently linked to the N- and/or C-termini of the tau additional residues. Similarly, where the tau peptide is KLDF (SEQ ID NO: 2), the linker is covalently linked to residues K and F, where the tau peptide is LDFK (SEQ ID NO: 3), the linker is covalently linked to residues L and K, and where the tau peptide is KLDFK (SEQ ID NO: 1), the linker is covalently linked to the residues K and K.

In an embodiment, the cyclic compound comprises a peptide comprising or consisting of KLDF (SEQ ID NO: 2), LDFK (SEQ ID NO: 3), or KLDFK (SEQ ID NO: 1) and a linker, wherein the linker is coupled to the N- and C-termini of the peptide.

In an embodiment, the cyclic peptide (or a linear peptide) is selected from a compound recited in Tables 2, 4 or 7, optionally wherein the cyclic compound is selected from cyclo(CGGKLDFKG) (SEQ ID NO: 31), cyclo(CGKLDFKG) (SEQ ID NO: 4), cyclo(CGGGGKLDFKG) (SEQ ID NO: 39), cyclo(CGKLDFKGG) (SEQ ID NO: 5), cyclo(CGGKLDFKGGGG) (SEQ ID NO: 34), cyclo(CGGGKLDFKG) (SEQ ID NO: 35), cyclo(CGKLDFG) (SEQ ID NO: 7), cyclo(CGGGKLDFG) (SEQ ID NO: 15), cyclo(CGGGGKLDFG) (SEQ ID NO: 19), cyclo(CGGGKLDFGG) (SEQ ID NO: 16 with linker 3,2), cyclo(CGGLDFKG) (SEQ ID NO: 52) or cyclo(CGLDFKGG) (SEQ ID NO: 49).

In an embodiment, the cyclic compound is cyclo(CGGKLDFKG) (SEQ ID NO: 31). In an embodiment, the cyclic compound is cyclo(CGKLDFKG) (SEQ ID NO: 4). In an embodiment, the cyclic compound is cyclo(CGGGGKLDFKG) (SEQ ID NO:39). In an embodiment, the cyclic compound is cyclo(CGKLDFKGG) (SEQ ID NO: 5). In an embodiment, the cyclic compound is cyclo(CGGKLDFKGGGG) (SEQ ID NO: 34). In an embodiment, the cyclic compound is cyclo(CGGGKLDFKG) (SEQ ID NO: 35). In an embodiment, the cyclic compound is cyclo(CGKLDFG) (SEQ ID NO: 7). In an embodiment, the cyclic compound is cyclo(CGGGKLDFG) (SEQ ID NO: 15). In an embodiment, the cyclic compound is cyclo(CGGGGKLDFG) (SEQ ID NO: 19). In an embodiment, the cyclic compound is cyclo(CGGGKLDFGG) (SEQ ID NO: 16). In an embodiment, the cyclic compound is cyclo(CGGLDFKG) (SEQ ID NO: 52). In an embodiment, the cyclic compound is cyclo(CGLDFKGG) (SEQ ID NO: 49).

Methods for making cyclized peptides are known in the art and include SS-cyclization or amide cyclization (head-to-tail, or backbone cyclization). Methods are further described in in the Example section. For example, a peptide with “C” residues at its N- and C-termini, e.g. CGGGKLDFGGC (SEQ ID NO: 64), can be reacted by SS-cyclization to produce a cyclic peptide. The cyclic compound can be synthesized as a linear molecule with the linker covalently attached to the N-terminus or C-terminus of the peptide comprising the tau peptide, optionally KLDF (SEQ ID NO: 2), LDFK (SEQ ID NO: 3) or related epitope e.g. comprising additional C and/or N terminal tau sequence, prior to cyclization. Alternatively, part of the linker is covalently attached to the N-terminus and part is covalently attached to the C-terminus prior to cyclization. In either case, the linear compound is cyclized for example in a head to tail cyclization (e.g. amide bond cyclization).

As described in the Examples, cyclic compounds were assessed for their relatedness to the conformational epitopes identified, and can be synthesized and used to prepare immunogens and used to raise antibodies selective for misfolded oligomeric tau. The epitopes KLDFK (SEQ ID NO: 1), KLDF (SEQ ID NO: 2), LDFK (SEQ ID NO: 3), as described herein may be a potential target in misfolded propagating strains of tau, and antibodies that recognize the conformational epitope may for example be useful in detecting such propagating strains.

As mentioned the above cyclic compounds comprising the tau peptides can be used as an immunogen for example to raise antibodies.

Accordingly, another aspect includes an immunogen (e.g. immunogenic compound) comprising cyclic compound described herein. In an embodiment, the immunogen comprises an immunogenicity enhancing agent such as Keyhole Limpet Hemocyanin (KLH). The immunogenicity enhancing agent can be coupled to the compound either directly, such as through an amide bound, or indirectly through a chemical linker. Alternatively, the immunogen may be a multi antigenic peptide (MAP).

The immunogen can be produced by conjugating the cyclic compound containing the constrained tau epitope peptide to an immunogenicity enhancing agent such as Keyhole Limpet Hemocyanin (KLH) or a carrier such bovine serum albumin (BSA) using for example the method described in Lateef et al 2007, herein incorporated by reference. The cyclic peptide can be conjugated to a protein carrier such as truncated rabies glycoprotein (MyBiosource Inc, San Diego, Calif.). In an embodiment, a method described in the Examples is used.

### III. Antibodies

Accordingly, the compounds and particularly the cyclic compounds comprising any 3 or 4 amino acid residues of KLDFK (SEQ ID NO: 1) such as tau peptide KLDF (SEQ ID NO: 2), LDFK (SEQ ID NO: 3) or KLDFK (SEQ ID NO:1) described herein, can be used to raise antibodies that selectively bind a cyclic compound comprising said tau peptide, and/or also bind misfolded forms of tau including misfolded oligomeric tau. The antibodies may selectively bind KLDF (SEQ ID NO: 2), LDFK (SEQ ID NO: 3), or KLDFK (SEQ ID NO:1) or a part thereof in misfolded oligomeric tau. As shown in the Examples, the cyclic compounds exhibit one or more spatial conformations that are dissimilar from monomeric tau, and which resemble partially unfolded or stressed fibrillar tau (biased tau). Further antibodies can be raised using said compounds that are expected to be selective for cyclic peptides and also bind misfolded oligomeric tau selectively. Similarly, cyclic compounds comprising for example KLDF (SEQ ID NO: 2), LDFK (SEQ ID NO: 3), and/or other related epitope sequences described herein can be used to raise antibodies that selectively bind an epitope in these residues accessible in the context of misfolded oligomeric tau.

Accordingly, the compounds and particularly the cyclic compounds described herein can be used to raise antibodies that selectively bind the epitope in tau that they comprise and/or which recognize specific conformations of these residues in tau, including one or more differential features described herein.

Accordingly, an aspect includes an antibody that selectively binds an epitope on tau, the epitope comprising or consisting of KLDF (SEQ ID NO: 2), LDFK (SEQ ID NO: 3), or KLDFK (SEQ ID NO: 1), a related epitope thereof such as a part thereof optionally a conformational epitope of any of the foregoing. In an embodiment, wherein when the epitope consists of KLDF (SEQ ID NO: 2), LDFK (SEQ ID NO: 3), or KLDFK (SEQ ID NO: 1) it is a conformational epitope.

In an embodiment, the antibody is a conformation selective antibody. In an embodiment, the antibody is a conformation selective KLDFK (SEQ ID NO:1) or part thereof binding antibody, such as a KLDF (SEQ ID NO: 2) or LDFK (SEQ ID NO: 3) binding antibody.

In an embodiment, the antibody is isolated.

In an embodiment, the antibody does not specifically bind monomeric tau. Selective binding can be measured using, for example, an ELISA or surface plasmon resonance measurement, as described herein.

Accordingly a further aspect is an antibody which selectively binds an epitope present on misfolded oligomeric tau (e.g. a conformational epitope), wherein the epitope comprises or consists of the sequence KLDF (SEQ ID NO: 2), LDFK (SEQ ID NO: 3), or KLDFK (SEQ ID NO: 1), or a part thereof.

In another embodiment, the epitope is a conformational epitope and consists of the amino acid sequence KLDF (SEQ ID NO: 2), LDFK (SEQ ID NO: 3), or KLDFK (SEQ ID NO: 1). In an embodiment, the antibody selectively binds KLDF (SEQ ID NO: 2), LDFK (SEQ ID NO: 3), or KLDFK (SEQ ID NO: 1) in a cyclic peptide, optionally wherein the linker is selected from any of GGCG (SEQ ID NO: 186; 1,2 linker), GCGG (SEQ ID NO: 43; 2,1 linker), GCG (1,1 linker), GCGGG (SEQ ID NO:44; 3,1 linker), GGCGGG (SEQ ID NO: 45; 3,2 linker), GGGCG (SEQ ID NO: 46; 1,3 linker), GGGGCGG (SEQ ID NO: 65; 2,4 linker) or GCGGGG (SEQ ID NO: 47; 4,1 linker) or any other linker described herein. For example, a linker GGCGGG (SEQ ID NO: 45) combined with epitope KLDF (SEQ ID NO: 2) would produce for example cyclo(CGGGKLDFGG) (SEQ ID NO:16).

In one embodiment, the antibody selectively binds a cyclic compound compared to the corresponding linear peptide. In an embodiment, the cyclic compound is cyclo(CGGKLDFKG) (SEQ ID NO: 31). In an embodiment, the cyclic compound is cyclo(CGKLDFKG) (SEQ ID NO: 4). In an embodiment, the cyclic compound is cyclo(CGGGGKLDFKG) (SEQ ID NO: 39). In an embodiment, the cyclic compound is cyclo(CGKLDFKGG) (SEQ ID NO: 5). In an embodiment, the cyclic compound is cyclo(CGGKLDFKGGGG) (SEQ ID NO: 34). In an embodiment, the cyclic compound is cyclo(CGGGKLDFKG) (SEQ ID NO: 35). In an embodiment, the cyclic compound is cyclo(CGKLDFG) (SEQ ID NO: 7). In an embodiment, the cyclic compound is cyclo(CGGGKLDFG) (SEQ ID NO: 15). In an embodiment, the cyclic compound is cyclo(CGGGGKLDFG) (SEQ ID NO: 19). In an embodiment, the cyclic compound is, cyclo(CGGGKLDFGG) (SEQ ID NO: 16). In an embodiment, the cyclic compound is cyclo(CGGLDFKG) (SEQ ID NO: 52). In an embodiment, the cyclic compound is cyclo(CGLDFKGG) (SEQ ID NO: 49).

In an embodiment, the antibody selectively binds a cyclic compound comprising an epitope peptide described herein comprising at least one alternate conformational feature described herein (e.g. of the epitope in a cyclic compound compared to a monomeric structural ensemble). For example, an antibody that binds a particular epitope conformation can be referred to as a conformation specific antibody. The conformation specific/selective antibody can differentially recognize a particular misfolded oligomeric tau species, and can have a higher affinity for one species or group of species compared to the monomeric species.

In an embodiment, the antibody selectively binds a cyclic compound comprising KLDF (SEQ ID NO: 2), LDFK (SEQ ID NO: 3), or KLDFK (SEQ ID NO: 1) or a part thereof, optionally in the context of cyclo(CGGGKLDFKG) (SEQ ID NO:35) or other cyclic peptide sequence listed in Table 2, 4 and/or 7 relative to an monomeric tau. For example, in an embodiment the antibody selectively binds KLDFK (SEQ ID NO: 1) in a cyclic conformation and has at least 1.5 fold, at least 2 fold, at least 2.5 fold, at least 3 fold at least 3.5 fold, at least 4 fold, at least 5 fold or more selective greater selectivity (e.g. binding affinity) for KLDFK (SEQ ID NO: 1) in the cyclic conformation compared to KLDFK (SEQ ID NO: 1) in a monomeric ensemble, for example as measured by ELISA or surface plasmon resonance, optionally using a method described herein.

In an embodiment, the antibody selectively binds a cyclic compound comprising the epitope relative to a monomeric ensemble or a species of tau for example, misfolded oligomeric tau polypeptide relative to native monomeric tau. In an embodiment, the selectivity is at least 1.5 fold, at least 2 fold, at least 2.5 fold, at least 3 fold at least 3.5 fold, at least 4 fold, at least 5 fold or more selective for the cyclic compound and/or misfolded oligomeric tau polypeptide over a species of tau selected from a monomeric ensemble of tau.

In an embodiment, the antibody was produced by immunizing with an immunogen comprising a cyclic peptide described herein. In an embodiment, the cyclic peptide (or a linear peptide) is selected from a compound recited in Tables 2, 4 or 7, optionally wherein the cyclic compound is selected from cyclo(CGGKLDFKG) (SEQ ID NO: 31; with linker 2,1), cyclo(CGKLDFKG) (SEQ ID NO: 4; with linker 1,1), cyclo(CGGGGKLDFKG) (SEQ ID NO:39; with linker 4,1), cyclo(CGKLDFKGG) (SEQ ID NO: 5; with linker 1,2), cyclo(CGGKLDFKGGGG) (SEQ ID NO: 34; with 3,2 linker), cyclo(CGGGKLDFKG) (SEQ ID NO: 35; with linker 3,1), cyclo(CGKLDFG) (SEQ ID NO: 7; with linker 1,1), cyclo(CGGGKLDFG) (SEQ ID NO: 15; with linker 3,1), cyclo(CGGGGKLDFG) (SEQ ID NO: 19; with linker 4,1), cyclo(CGGGKLDFGG) (SEQ ID NO: 16; with linker 3,2), cyclo(CGGLDFKG) (SEQ ID NO: 52; with linker 2,1) or cyclo(CGLDFKGG) (SEQ ID NO: 49; with linker 1, 2).

In an embodiment, the antibody is selected from an antibody having a variable region of a clone as recited in Table 10 and/or having CDR sequences (e.g. a set of CDR sequences) as recited in Table 11.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 95
GFNIKDTH;
CDR-H2:
SEQ ID NO: 96
IDPSNGNT;
CDR-H3:
SEQ ID NO: 97
ATGFAY;
CDR-L1:
SEQ ID NO: 98
GNIHNY;
CDR-L2:
SEQ ID NO: 99
NAK;
and
CDR-L3:
SEQ ID NO: 100
QHFWYTPWT.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 101
GYAFSSYW;
CDR-H2:
SEQ ID NO: 102
IYPGDGDT;
CDR-H3:
SEQ ID NO: 103
ASQIYDGYYTFTY;
CDR-L1:
SEQ ID NO: 104
QSLLNSRTRKNY;
CDR-L2:
SEQ ID NO: 105
WAS;
and
CDR-L3:
SEQ ID NO: 106
KQSYNLWT.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 107
GYTFTNYG;
CDR-H2:
SEQ ID NO: 108
INTYSGEP;
CDR-H3:
SEQ ID NO: 109
ARSPGAYYTLDY;
CDR-L1:
SEQ ID NO: 110
QSLLNSRTRKNY;
CDR-L2:
SEQ ID NO: 111
WAS;
and
CDR-L3:
SEQ ID NO: 112
KQSYNLYT.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 113
GYTFTNYG;
CDR-H2:
SEQ ID NO: 114
INTYTGEP;
CDR-H3:
SEQ ID NO: 115
GRGIRDYYTMDY;
CDR-L1:
SEQ ID NO: 116
QSLLNNRTRKNY;
CDR-L2:
SEQ ID NO: 117
WAS;
and
CDR-L3:
SEQ ID NO: 118
KQSYNLYT.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 119
GYSITSDYA;
CDR-H2:
SEQ ID NO: 120
ISYSGST;
CDR-H3:
SEQ ID NO: 121
AAYYRYGLAYFAY;
CDR-L1:
SEQ ID NO: 122
QSLLDSDGKTY;
CDR-L2:
SEQ ID NO: 123
Lvs;
and
CDR-L3:
SEQ ID NO: 124
WQGTHFPQT.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 125
GYTFTNFG;
CDR-H2:
SEQ ID NO: 126
INTFTGEP;
CDR-H3:
SEQ ID NO: 127
ARSPGRVYTLDY;
CDR-L1:
SEQ ID NO: 128
QSLLNSRTRKNY;
CDR-L2:
SEQ ID NO: 129
WAS;
and
CDR-L3:
SEQ ID NO: 130
KQSYNLYT.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 131
GYRFTSYW;
CDR-H2:
SEQ ID NO: 132
IYPGNSDT;
CDR-H3:
SEQ ID NO: 133
TRPYFDS;
CDR-L1:
SEQ ID NO: 134
QSLLDSDGKTY;
CDR-L2:
SEQ ID NO: 135
LVS; and
CDR-L3:
SEQ ID NO: 136
WQGTHFPQT.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 137
GFSITSDYA;
CDR-H2:
SEQ ID NO: 138
IRYSGNT;
CDR-H3:
SEQ ID NO: 139
ASTLEDSYWYFDV;
CDR-L1:
SEQ ID NO: 140
QSIVHTNGNTY;
CDR-L2:
SEQ ID NO: 141
KVS;
and
CDR-L3:
SEQ ID NO: 142
FQGSHVPLT.

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 143
GYTFTSYY;
CDR-H2:
SEQ ID NO: 144
INPSNGGS;
CDR-H3:
SEQ ID NO: 145
TRGAF;
CDR-L1:
SEQ ID NO: 146
QSLLDSDRKTY;
CDR-L2:
SEQ ID NO: 147 (123)
Lvs;
and
CDR-L3:
SEQ ID NO: 148
WQVTHFPHT;

In an embodiment, the antibody described herein comprises a heavy chain variable region and a light chain variable region, the heavy chain variable region comprising complementarity determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:

CDR-H1:
SEQ ID NO: 149
GFSLSTSGMG;
CDR-H2:
SEQ ID NO: 150
IWWDDDK;
CDR-H3:
SEQ ID NO: 151
VRSIYYYDSSPYYYVMDY;
CDR-L1:
SEQ ID NO: 152
QDVSIA;
CDR-L2:
SEQ ID NO: 153
SAS;
and
CDR-L3:
SEQ ID NO: 154
QQHYSSPLT.

In an embodiment, the antibody described herein comprises a heavy chain variable region and a light chain variable region, the amino acid sequences of said heavy chain variable region and light chain variable region comprising the sequences of SEQ ID NOs: 75 and 76; SEQ ID NOs: 77 and 78; SEQ ID NOs: 79 and 80; SEQ ID NOs: 81 and 82; SEQ ID NOs: 83 and 84; SEQ ID NOs: 85 and 86; SEQ ID NOs: 87 and 88; SEQ ID NOs: 89 and 90; SEQ ID NOs: 91 and 92; or SEQ ID NOs: 93 and 94, respectively, or having an amino acid sequence with at least 80%, at least 90%, or at least 95% sequence identity with the sequences of SEQ ID NOs: 75 and 76; SEQ ID NOs: 77 and 78; SEQ ID NOs: 79 and 80; SEQ ID NOs: 81 and 82; SEQ ID NOs: 83 and 84; SEQ ID NOs: 85 and 86; SEQ ID NOs: 87 and 88; SEQ ID NOs: 89 and 90; SEQ ID NOs: 91 and 92; or SEQ ID NOs: 93 and 94 wherein the CDR sequences are as underlined in Table 10.

To produce monoclonal antibodies, antibody producing cells (lymphocytes) can be harvested from a subject immunized with an immunogen described herein, and fused with myeloma cells by standard somatic cell fusion procedures thus immortalizing these cells and yielding hybridoma cells including the methods described herein. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121: 140-67 (1986)), and screening of combinatorial antibody libraries (Huse et al., Science 246:1275 (1989)). Hybridoma cells can be screened immunochemically for production of antibodies selectively reactive with the desired epitopes and the monoclonal antibodies can be isolated.

Specific antibodies, or antibody fragments, reactive against particular antigens or molecules, may also be generated by screening expression libraries encoding immunoglobulin genes, or portions thereof, expressed in bacteria with cell surface components. For example, complete Fab fragments, VH regions and FV regions can be expressed in bacteria using phage expression libraries (see for example Ward et al., Nature 41:544-546 (1989); Huse et al., Science 246:1275-1281 (1989); and McCafferty et al., Nature 348:552-554 (1990).

The antibody sequences including the CDRs can be determined by sequence analysis of immunoglobulin transcripts obtained from the monoclonal antibody producing hybridoma.

Additionally, antibodies specific for the epitopes described herein are readily isolated by screening antibody phage display libraries. For example, an antibody phage library is optionally screened by using a disease specific epitope of the current disclosure to identify antibody fragments specific for the disease specific epitope. Antibody fragments identified are optionally used to produce a variety of recombinant antibodies that are useful with different embodiments of the present disclosure. Antibody phage display libraries are commercially available, for example, through Xoma (Berkeley, Calif.) Methods for screening antibody phage libraries are well known in the art.

In one embodiment, the antibody is a single chain antibody. In one embodiment, the antibody is a humanized antibody. In yet another embodiment, the antibody is a single chain humanized antibody.

Also provided is an immunoconjugate comprising an antibody described herein and for example a detectable label. Such antibodies can be used for example to detect pathogenic species in vivo or to detect pathogenic tau in a sample such as blood or a fraction thereof or CSF. For example, such antibodies can be used to determine drug efficacy and/or target engagement in a clinical trial by determining the level of pathogenic tau.

### IV. Nucleic Acids and Cells

A further aspect is an isolated nucleic acid comprising a sequence encoding an antibody or part described herein. For example, the isolated nucleic acid comprises a sequence encodes a heavy chain or a light chain variable region comprising the CDRs (e.g. a set as shown therein) as shown in Table 11.

In an embodiment, the nucleic acid comprises a sequence that encodes an antibody or part thereof described herein (e.g. heavy chain variable domain, light chain variable domain etc). In one embodiment, the nucleic acid comprises a sequence that encodes the light chain variable domain of any one of SEQ ID Nos: 76, 78, 80, 82, 84, 86, 88, 90, 92 or 94, or a sequence with at least 70%, 80%, 85%, 90%, 95%, 98% or 99% sequence identity to any one of SEQ ID Nos: 76, 78, 80, 82, 84, 86, 88, 90, 92 or 94. In an embodiment, the nucleic acid that encodes a light chain variable domain comprises the sequence of any one of SEQ ID NOs: 156, 158, 160, 162, 164, 166, 168, 170, 172, or 174, or a sequence with at least 70%, 80%, 85%, 90%, 95%, 98% or 99% sequence identity to any one of SEQ ID Nos: 156, 158, 160, 162, 164, 166, 168, 170, 172, or 174. In one embodiment, the nucleic acid comprises a sequence that encodes the heavy chain variable domain of any one of SEQ ID Nos: 75, 77, 79, 81, 83, 85, 87, 89, 91, or 93, or a sequence with at least 70%, 80%, 85%, 90%, 95%, 98% or 99% sequence identity to any one of SEQ ID Nos: 75, 77, 79, 81, 83, 85, 87, 89, 91, or 93. In an embodiment, the nucleic acid that encodes a light chain variable domain comprises the sequence of any one of SEQ ID NOs: 155, 157, 159, 161, 163, 165, 167, 169, or 173, or a sequence with at least 70%, 80%, 85%, 90%, 95%, 98% or 99% sequence identity to any one of SEQ ID Nos: 155, 157, 159, 161, 163, 165, 167, 169, or 173.

Such nucleic acids that comprise a sequence that encodes either the heavy or the light chain can be used for example to produce single chain antibodies.

In other embodiments, the nucleic acid encodes a single chain antibody. In some embodiments, the nucleic acid comprises a sequence that encodes the light chain variable domain of any one of SEQ ID Nos: 76, 78, 80, 82, 84, 86, 88, 90, 92 or 94, or a sequence with at least 70%, 80%, 85%, 90%, 95%, 98% or 99% sequence identity to any one of SEQ ID Nos: 76, 78, 80, 82, 84, 86, 88, 90, 92 or 94 and a sequence that encodes the heavy chain variable domain of any one of SEQ ID Nos: 75, 77, 79, 81, 83, 85, 87, 89, 91 or 93, or a sequence with at least 70%, 80%, 85%, 90%, 95%, 98% or 99% sequence identity to any one of SEQ ID Nos: 75, 77, 79, 81, 83, 85, 87, 89, 91, or 93, wherein said encoded antibody binds oligomeric tau and/or a cyclic compound described herein. In one embodiment the nucleic acid comprises sequences that encode the heavy and light chain variable sequences of the antibodies recited in Table 10 and/or having CDR sequences as recited in Table 11. In one embodiment, the nucleic acid comprises the sequences SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 167 and 168; SEQ ID NOs: 169 and 170; SEQ ID NOs: 171 and 172; or SEQ ID NOs: 173 and 174, or sequences with at least 70%, 80%, 85%, 90%, 95%, 98% or 99% sequence identity to SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 167 and 168; SEQ ID NOs: 169 and 170; SEQ ID NOs: 171 and 172; or SEQ ID NOs: 173 and 174, wherein the encoded antibody binds oligomeric tau and/or a cyclic compound described herein.

Described in Table 12 are the nucleic acid sequences that encode the variable domains described in Table 10. In an embodiment, the nuclide acid comprises a nucleic acid encoding a sequence encoding the variable domains are also provided

In an embodiment, the nucleic acid comprising a sequence that encodes an antibody or part thereof further comprises a sequence encoding a secretion signal peptide. The secretion signal peptide can be the native secretion signal peptide or a non-native signal secretion signal peptide.

In one embodiment, the nucleic acid comprises a sequence encoding a secretion signal peptide. For example, the secretion signal peptide can be a native heavy chain signal peptide or a native light chain signal peptide. Exemplary heavy chain signal sequences include/comprise METGLRWLLLVAVLKGVQCQ (SEQ ID NO: 175), MELGLSWIFLLAILKGVQC (SEQ ID NO: 176), MELGLRWVFLVAILEGVQC (SEQ ID NO: 177), MKHLWFFLLLVAAPRWVLS (SEQ ID NO: 178), MDWTWRILFLVAAATGAHS (SEQ ID NO: 179), MDWTWRFLFVVAAATGVQS (SEQ ID NO: 180), MEFGLSWLFLVAILKGVQC (SEQ ID NO: 181), MEFGLSWVFLVALFRGVQC (SEQ ID NO: 182) or MDLLHKNMKHLWFFLLLVAAPRWVLS (SEQ ID NO: 183). Exemplary light chain signal sequences include MDMRVPAQLLGLLLLWLSGARC (SEQ ID NO: 184) or MKYLLPTAAAGLLLLAAQPAMA (SEQ ID NO: 185).

The nucleic acid may also comprise a sequence encoding a detectable tag, for example a commonly used purification tag or detection tag such as HA, FLAG, or MYC.

The sequence may be codon optimized, for example codon optimized for expression in human cells.

Another aspect is an expression cassette or vector comprising the nucleic acids herein described. The expression cassette can comprise for example the nucleic acid encoding the antibody, optionally a single chain antibody, and regulatory sequences such as a promoter that is operatively linked to the nucleic acid. In an embodiment, the vector is an isolated vector.

The vector can be any vector, suitably an expression vector suitable for producing a single chain antibody described herein. In an embodiment, the vector is suitable for expressing for example single chain antibodies (e.g. intrabodies).

The nucleic acid molecules may be incorporated in a known manner into an appropriate expression vector which ensures expression of the protein.

Possible expression vectors include but are not limited to cosmids, plasmids, or modified viruses (e.g. replication defective retroviruses, including lentiviral vectors, adenoviruses and adeno-associated viruses).

In one embodiment, the vector is an adeno associated virus capable of transducing neuronal cells (e.g. AAV serotype 9).

The vectors may comprise suitable regulatory sequences.

Suitable regulatory sequences may be derived from a variety of sources, including bacterial, fungal, viral, mammalian, or insect genes. Examples of such regulatory sequences include: a transcriptional promoter and enhancer or RNA polymerase binding sequence, a ribosomal binding sequence, including a translation initiation signal. Additionally, depending on the cell to be transfected/infected/transduced and the vector employed, other sequences, such as an origin of replication, additional DNA restriction sites, enhancers, and sequences conferring inducibility of transcription may be incorporated into the expression vector. In an embodiment, the regulatory sequences direct or increase expression in neural tissue and/or cells. In an embodiment, the vector is a viral vector. The recombinant expression vectors may also contain a marker gene which facilitates the selection of host cells transformed, infected or transfected with a vector for expressing an antibody described herein. The recombinant expression vectors may also contain other expression cassettes which encode for example a fusion moiety or detectable label (e.g. for creating an antibody “fusion protein”) which can aid in the detection, including for example tags or labels described herein.

The nucleic acids or vectors can be used to produce an antibody or part thereof described herein or deliver said antibody or binding fragment, optionally wherein the antibody is a single chain antibody, in a cell, for example for intracellular expression in a cell in a subject, or to a subject.

A wide range of approaches to transduce the cells can be used, including viral vectors, “naked” DNA, DNA in lipid or other nanoparticles, adjuvant assisted DNA, gene gun etc. For example, retroviral vectors such as lentiviral vectors can also be used to transduce cells. Other vector systems useful in practicing aspects of the present invention include adenoviral or adeno associated virus based vectors.

Also provided in another aspect is a cell expressing an antibody described herein. In an embodiment, the cell is an isolated and/or recombinant cell, expressing an antibody described herein or comprising a vector herein disclosed. In an embodiment, the cell is a fused cell such as a hybridoma. In an embodiment, the cell is a mammalian cell such as a CHO cell, an HEK-293 cell. In an embodiment, the cell is an insect cell such as Sf9, Sf21, Tni, or S2.

### V. Compositions

A further aspect is a composition comprising a cyclic compound, immunogen, immunoconjugate, nucleic acid, vector or antibody described herein.

In an embodiment, the composition comprises a diluent.

Suitable diluents for polypeptides, including antibodies or fragments thereof and/or cells include but are not limited to saline solutions, pH buffered solutions and glycerol solutions or other solutions suitable for freezing polypeptides and/or cells.

Suitable diluents for nucleic acids or vectors include but are not limited to water, saline solutions or ethanol.

The composition can comprise lipid particles such as liposomes, nanoparticles, or nanosomes for aiding delivering the nucleic acid and/or vectors.

In an embodiment, the composition comprises a nucleic acid or vector described herein. In another embodiment, the composition comprises an antibody or part thereof described herein and a diluent. In an embodiment, the composition is a sterile composition.

The composition can be formulated for intrathecal, intraparenchymal or intraventricular administration.

In an embodiment, the composition comprises a pharmaceutically acceptable carrier, diluent, and/or excipient. In an embodiment, the composition is for a method described herein.

The composition can comprise one or more antibodies, immunoconjugates, cyclic compounds, immunogens, cells, nucleic acids or vectors described herein. For example, the composition can comprise 2, 3, 4, or more antibodies or binding fragments described herein; 2, 3, 4, or more immunoconjugates described herein; 2, 3, 4, or more cyclic compounds described herein; 2, 3, 4, or more immunogens described herein; 2, 3, 4, or more cells described herein; 2, 3, 4, or more nucleic acids described herein or 2, 3, 4, or more vectors, described herein.

Compositions comprising for example a cyclic compound, immunogen or combinations of any thereof (e.g. including multiple cyclic compounds, immunogens or mixtures thereof) are immunogenic and induce production of antibody, for example antibody selective for oligomeric tau. Accordingly, an aspect provides immunogenic compositions comprising a cyclic compound, immunogen or combinations of any thereof for example 2, 3, 4, or more cyclic compounds; 2, 3, 4 or more immunogens; or mixtures of any thereof.

In an embodiment comprising a compound or immunogen described herein, the composition comprises an adjuvant.

Adjuvants that can be used for example, include Intrinsic adjuvants (such as lipopolysaccharides) normally are the components of killed or attenuated bacteria used as vaccines. Extrinsic adjuvants are immunomodulators which are typically non-covalently linked to antigens and are formulated to enhance the host immune responses. Aluminum hydroxide, aluminum sulfate and aluminum phosphate (collectively commonly referred to as alum) are routinely used as adjuvants. A wide range of extrinsic adjuvants can provoke potent immune responses to immunogens. These include saponins such as Stimulons (QS21, Aquila, Worcester, Mass.) or particles generated therefrom such as ISCOMs and immunostimulating complexes and ISCOMATRIX, complexed to membrane protein antigens immune stimulating complexes, pluronic polymers with mineral oil, killed mycobacteria or mineral oil, Freund's complete adjuvant, bacterial products such as muramyl dipeptide (MDP) or lipopolysaccharide (LPS), as well as lipid A, or liposomes.

In an embodiment, the adjuvant is aluminum hydroxide. In another embodiment, the adjuvant is aluminum phosphate. Adjuvants with mucoadhesive characteristics include, but are not limited to, polymers, such as those comprising Carbopols or acrylic acids (such as polyacrylic acids), such as Carbigen™ adjuvant; oil-in-water based adjuvants, such as Emulsigen® adjuvant; nanoparticles; or combinations thereof.

Oil in water emulsions include squalene; peanut oil; MF59 (WO 90/14387); SAF (Syntex Laboratories, Palo Alto, Calif.); or Ribi™ (Ribi Immunochem, Hamilton, Mont.) as well as Emulsigen (Phibro Animal Health Corp, Teaneck, N.J.). Oil in water emulsions may be used with immunostimulating agents such as muramyl peptides (for example, N-acetylmuramyl-L-threonyl-D-isoglutamine (thr-MDP), -acetyl-normuramyl-L-alanyl-D-isoglutamine (nor-MDP), N-acetylmuramyl-L-alanyl-D-isoglutamyl-L-alanine-2-(1′-2′dipalmitoyl-sn-glycero-3-hydroxyphosphoryloxy)-ethylamine (MTP-PE), N-acetylglucsaminyl-N-acetylmuramyl-L-AI-D-isoglu-L-Ala-dipalmitoxy propylamide (DTP-DPP) Theramide™), or other bacterial cell wall components.

The adjuvant may be administered with an immunogen as a single composition. Alternatively, an adjuvant may be administered before, concurrent and/or after administration of the immunogen.

In an embodiment, the composition comprises an antibody described herein. In another embodiment, the composition comprises an antibody described herein and a diluent. In an embodiment, the composition is a sterile composition.

The term compound as used herein can refer for example to the peptide, immunogen, antibody, immunoconjugate etc.

Another aspect includes an antibody complex comprising an antibody described herein and tau (e.g. misfolded tau oligomers or soluble fibrils). The complex may be in solution.

### VI. Kits

A further aspect relates to a kit comprising i) an antibody and/or binding fragment thereof, or immunoconjugate comprising said antibody ii) a nucleic acid, iii) cyclic and/or linear peptide or immunogen, iv) composition or v) recombinant cell described herein, comprised in a vial such as a sterile vial or other housing and optionally a reference agent and/or instructions for use thereof.

### VII. Methods

Included are methods for making the compounds, immunogens, nucleic acids, vectors, immunoconjugates and antibodies described herein.

In particular, provided are methods of making an antibody selective for a conformational epitope of KLDF (SEQ ID NO: 2), LDFK (SEQ ID NO: 3), KLDFK (SEQ ID NO: 1) or related epitope. The method can comprise one or more steps described in the Examples for making the current antibodies. For example, the method can comprise administering a subject with an a cyclic compound or immunogen described herein, isolating B cells, preparing hybridoma cell lines or cloning B cells; and testing the antibody produced by the cell line for specificity for the tau peptide of the immunogen or oligomeric tau, for example identifying antibodies that preferentially bind tau peptide presented in a cyclic compound relative to a linear compound and/or identifying antibodies that preferentially bind the tau peptide in oligomeric tau relative to non-oligomeric tau (e.g. monomeric tau). In some embodiments, the antibody sequence is determined and antibodies or fragments thereof are synthesized

Antibody libraries can also be screened using the cyclic compounds described herein and an antibody with suitable properties selected. Suitable properties include one or more of the antibody properties described herein.

A further aspect provides a method of detecting whether a test sample comprises misfolded oligomeric tau.

In an embodiment, the method comprises:

a. contacting the test sample with the antibody or immunoconjugate described herein under conditions permissive to produce an antibody: misfolded oligomeric tau polypeptide complex; and

b. detecting the presence of any complex;

wherein the presence of detectable complex is indicative that the test sample may contain misfolded oligomeric tau polypeptide.

In another embodiment, the method comprises:
a. contacting a test sample of said subject with an antibody or immunoconjugate described herein, under conditions permissive to produce an antibody-antigen complex;
b. measuring the amount of the antibody-antigen complex in the test sample; and
c. comparing the amount of antibody-antigen complex in the test sample to a control;

wherein detecting antibody-antigen complex in the test sample as compared to the control indicates that the sample comprises misfolded oligomeric tau.

In an embodiment, the test sample is a biological sample. In an embodiment, the test sample comprises brain tissue or an extract thereof, saliva, blood, and/or cerebrospinal fluid (CSF). In an embodiment, the test sample is obtained from a human subject.

In some embodiments, the test sample is from a subject with comprising a genetic mutation in the tau gene.

In another embodiment, the test sample is from a subject with or suspected of having a tauopathy. For example, the tauopathy is Alzheimer's disease (AD), Pick's disease, frontotemporal dementia or frontotemporal lobar degeneration, progressive supranuclear palsy, corticobasal degeneration, primary age-related tauopathy, chronic traumatic encephalopathy, subacute sclerosing panencephalitis, frontotemporal dementia or parkinsonism linked to chromosome 17.

A number of methods can be used to determine if misfolded oligomeric tau polypeptides is present in a test sample using the antibodies described herein, including immunoassays such as flow cytometry, dot blot, Western blots, ELISA, or immunoprecipitation followed by SDS-PAGE immunocytochemistry or other detection platform (e.g. SIMOA, MSD, etc).

As described in the Examples surface plasmon resonance can be used to assess conformation specific binding.

A labelled antibody or immunoconjugate described herein can also be administered to a subject to detect the location of misfolded tau. The measuring may for example by immunofluorescence or PET tracer.

The methods may also include colocalization staining for example pan-tau staining.

A further aspect includes a method of inducing an immune response in a subject, comprising administering to the subject a compound, immunogen, nucleic acid or vector or a composition comprising any of the foregoing as described herein. In some embodiments, the method comprises isolating cells and/or antibodies that specifically bind the compound or immunogen administered. The isolated antibodies can be tested using one or more assays described in the Examples.

As described in the Examples, the ability of antibodies to inhibit or reduce tau PFF-induced formation of intracellular tau aggregates was determined using a cellular Fluorescence Energy Resonance Transfer (FRET) assay. As reported by Holmes, B B et al, proteopathic tau seeding activity in the FRET assay is an “early and robust marker of tauopathy” in a mouse model. Inhibition of seeding by an antibody, as observed herein, would therefore be expected to inhibit tau pathogenesis.

Accordingly, a further aspect includes a method of reducing or inhibiting tau aggregation/aggregates and/or propagation, comprising contacting a cell or tissue with—or administering to a cell or tissue—the cell or tissue comprising misfolded oligomeric tau polypeptide and/or soluble fibrils, an antibody, cyclic compound, immunogen, immunoconjugate, nucleic acid or vector herein disclosed.

In an embodiment, the cell or tissue is in vitro. In another embodiment, the cell or tissue is in vivo. For example, the cell or tissue is in a subject, optionally a human subject. For example, the cell is a brain cell. For example, the tissue is a brain tissue extract and/or cerebrospinal fluid (CSF).

Another aspect herein disclosed relates to a method of treating a tauopathy in a subject in need thereof, comprising administering to the subject an effective amount of an antibody, cyclic compound, immunogen, immunoconjugate, nucleic acid or vector herein disclosed or a composition comprising said antibody, cyclic compound, immunogen, immunoconjugate, nucleic acid or vector. The antibody, cyclic compound, immunogen, immunoconjugate, nucleic acid or vector can be administered into the CNS e.g. via an intrathecal, intraparenchymal or intracerebroventricualr route of administration, or peripherally e.g. via intravenous, intramuscular, intradermal or subcutaneous routes of administration. For example, vectorized antibody can be delivered into the CNS or peripherally.

In an embodiment, the tauopathy is Alzheimer's disease (AD), Pick's disease, frontotemporal dementia or frontotemporal lobar degeneration, progressive supranuclear palsy, corticobasal degeneration, primary age-related tauopathy, chronic traumatic encephalopathy, subacute sclerosing panencephalitis, frontotemporal dementia or parkinsonism linked to chromosome 17.

In an embodiment, the subject is a human subject.

The nucleic acid or vector can for example be comprised in a composition as described herein for example in combination with a pharmaceutically acceptable carrier, diluent and/or excipient and/or formulated for example in nanoparticles, or nanosomes for aiding delivering the nucleic acid and/or vectors.

The compositions, antibodies, immunoconjugates, nucleic acids and/or vectors described herein can be administered for example, by parenteral, intravenous, subcutaneous, intramuscular, intracranial, intraventricular, intrathecal, intraorbital, ophthalmic, intraspinal, intracisternal, intraperitoneal, intranasal, aerosol or oral administration.

Other embodiments contemplate the co-administration of the compositions, antibodies, immunoconjugates, nucleic acid and/or vectors described herein with biologically active molecules known to facilitate the transport across the blood brain barrier.

Also contemplated in certain embodiments, are methods for administering the compositions, antibodies, immunoconjugates, nucleic acids and/or vectors described herein across the blood brain barrier such as those directed at transiently increasing the permeability of the blood brain barrier as described in U.S. Pat. No. 7,012,061 “Method for increasing the permeability of the blood brain barrier”, herein incorporated by reference.

Also contemplated herein is the viral delivery of a nucleic acid or vector described herein for expression of one or more antibodies described herein, in a subject in need thereof. An aspect includes a method of treating a subject comprising administering to a subject in need thereof an effective amount of a vectorized antibody of the disclosure described herein, or a composition comprising said vectorized antibody, optionally in combination with another tauopathy treatment. In one embodiment, the vectorized antibody is a viral vector comprising a nucleic acid encoding an antibody described therein. In one embodiment, the method is for intracellular expression of an intrabody in a subject in need thereof.

For example, viral vectors such as adeno-associated virus (AAV, for example AAV9) or lentiviral vectors etc can used. Non-viral vectors can also be used. In certain embodiments, the nucleic acid, vector or composition can be injected intraventricularly or intrathecally. In other embodiments, the nucleic acid, vector or composition could be administered intravenously or subcutaneously or intramuscularly using for example a depot for sustained production of secreted single chain antibody.

The antibody, cyclic compound, immunogen, immunoconjugate, nucleic acid or vector herein disclosed may be administered to the subject in need thereof as part of a combination treatment against the tauopathy. In an embodiment, the method of treating comprises administering to the subject an effective amount of an antibody herein disclosed with an additional treatment.

In an embodiment, the additional treatment is an additional antibody, an antidepressant (e.g. a selective serotonin reuptake inhibitor), an antipsychotic, levodopa, a dopamine agonist, and/or mixtures thereof. For example, the additional antibody is an antibody that binds amyloid-beta as described in International Patent Publication Nos. WO 2017/079833, WO 2017/079834, WO 2017/079831, WO 2017/079832 or WO 2017/079835, each of which are hereby incorporated herein by reference in their entirety.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

Molecular-dynamics-based simulations which impose a global coordinate bias on a protein (or peptide-aggregate) to force the protein (or peptide-aggregate) to misfold and then predict the most likely unfolded regions of the partially unstructured protein (or peptide aggregate) were used to identify epitopes that are selectively or preferentially displayed in misfolded oligomeric tau. Biasing simulations were performed and the change in solvent accessible surface area (SASA) corresponding to each residue was measured (compared to that of the initial fibril structure of the protein under consideration). SASA represents the surface area that is accessible to H₂O. A positive change in SASA (compared to that of the initial structure of the protein under consideration) may be considered to be indicative of unfolding in the region of the associated residue index. Two other methods were used in addition to SASA to identify candidate epitopes. These were the loss of fibril contacts, defined by non-hydrogen atoms within a cut-off length, and root mean squared fluctuations (RMSF), measuring the extent of deviations about the average in a structural ensemble; here an increase in RMSF for some amino acids indicates an increase in the dynamics of those amino acids.

The Lipari-Szabo S² order parameter often used in NMR [J. Am. Chem. Soc., 1982, 104 (17), pp 4546-4559, DOI: 10.1021/ja00381a009] may be used as a substitute order parameter for RMSF in order to identify epitopes.

The methods were applied to the tau fibril (PDB entry 5O3L).

A structure of 10 chains of tau fibril has been determined and is listed on the protein databank as PDB entry 5O3L. The PDB 5O3L structure, any part of it, or the whole sequence of each chain extended by e.g. 10 amino acids on both N- and C-termini, can be equilibrated on a computer to obtain an equilibrium ensemble, which was used for all measurements of the fibril conformations of the epitopes in the fibril structure of tau, referred to herein variably as “structured fibril” or “unbiased fibril structure of tau”, “fibril ensemble of tau”, “equilibrium fibril ensemble of tau”, or “tau fibril structural ensemble”.

The monomer ensemble can be obtained for example by first taking as a starting structure one of the chains from the PDB fibril (5O3L). As tau is a large protein, a portion comprising tau was assessed. Residues 296 to 388 of human tau were used in the assessments.

A Pivot algorithm is then implemented 20 times to induce large conformational changes to the configuration, thus generating a new randomized configuration. The pivot algorithm is then run again on this configuration 20 times, to generate another randomized configuration, and so on, to generate multiple different unfolded structures to be used as initial configurations for molecular dynamics (MD) simulations. For each of these initial structures, we then perform a 3 ns equilibration simulation. For some of these simulations we have collected a snapshot every 1 ns. For other simulations, we have collected the snapshot configuration at the end of the simulation. We have found that the correlation time in these MD simulations is generally less than 1 ns, so both of the above methods are acceptable for generating an equilibrium ensemble. All of the snapshots were accumulated to generate a monomer ensemble with 7166 configurations (KLDFK (SEQ ID NO: 1) and LDFK (SEQ ID NO: 3)) or 5500 configurations (KLDF SEQ ID NO: 2).

Simulations were performed for the initial fibril structure using the collective coordinates method as described in International Patent Publication No. WO 2017/079836 and the CHARMM force-field parameters described in: K. Vanommeslaeghe, E. Hatcher, C. Acharya, S. Kundu, S. Zhong, J. Shim, E. Darian, O. Guvench, P. Lopes, I. Vorobyov, and A. D. Mackerell. Charmm general force field: A force field for drug-like molecules compatible with the CHARMM all-atom additive biological force fields. Journal of Computational Chemistry, 31(4):671-690, 2010; and P. Bjelkmar, P. Larsson, M. A. Cuendet, B. Hess, and E. Lindahl. Implementation of the CHARMM force field in GROMACS: analysis of protein stability effects from correlation maps, virtual interaction sites, and water models. J. Chem. Theo. Comp., 6:459-466, 2010, both of which are hereby incorporated herein by reference, with TIP3P water as solvent. The collective coordinate method applies a global bias to the fibril structure in order to induce a partially disordered fibril structure with 60% of the original contacts. Contacts are defined by non-hydrogen Adams within a cut off distance as described in WO/2017/079836.

The partially disordered fibril structure is maintained to have 60% of the original contacts for 100 ns. This is repeated 10 times as described in WO/2017/079836. The last 49 ns of these simulations are used to obtain snapshot conformations. The total simulation time from which snapshots are taken is approximately 490 ns. 4200 snapshots are uniformly sampled to generate the stressed or biased fibril ensemble.

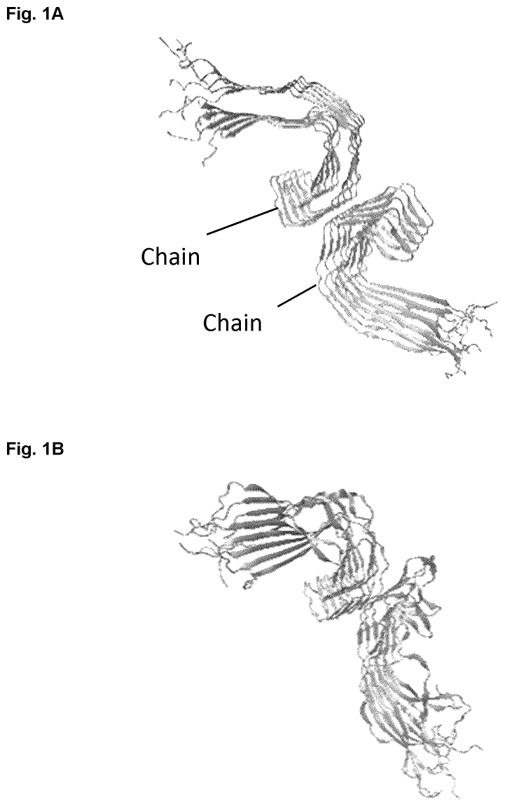

A representation of the PDB 5O3L structure is shown in FIG. 1. FIG. 1A is schematic representation of tau comprising 10 chains as shown in PDB 5O3L and FIG. 1A is a schematic representation of tau comprising 10 chains after collective coordinate biasing to partially disorder the fibril structure.

A 30 ns MD simulation was run starting from the structure in PDB 5O3L. From this simulation, 3010 snapshots are uniformly sampled, to obtain the fibril ensemble.

Analysis identified epitope sequences predicted to be preferentially accessible in the stressed fibril.

Epitope Predictions

Analysis of all 10 chains in the stressed fibril identified several regions prone to unfolding according to the Collective coordinates method.

Amino acid stretches 344-346, 341-349, 342-350, 344-346, 343-346, 344-347, 345-347, 343-352 and 345-347 were identified as regions prone to unfolding under biasing pressure and candidate regions accessible in misfolded oligomeric tau. The criteria assessed was solvent accessible surface area (SASA), where SASA identifies regions that are more accessible, for example to antibody binding and less likely to buried in the protein.

Amino acid stretches 337-346, 343-350, 341-346, 342-345, 341-345, 346-348 and 343-345 were identified as regions prone to unfolding under biasing pressure and candidate regions accessible in misfolded oligomeric tau. The criteria assessed was number of fibril contacts, where loss of fibril contacts identifies a region prone to unfolding.

The epitopes KLDF (SEQ ID NO: 2), LDFK (SEQ ID NO: 3), and KLDFK (SEQ ID NO: 1) emerge as predicted epitopes from the PDB structure 5O3L using the collective coordinates approach.

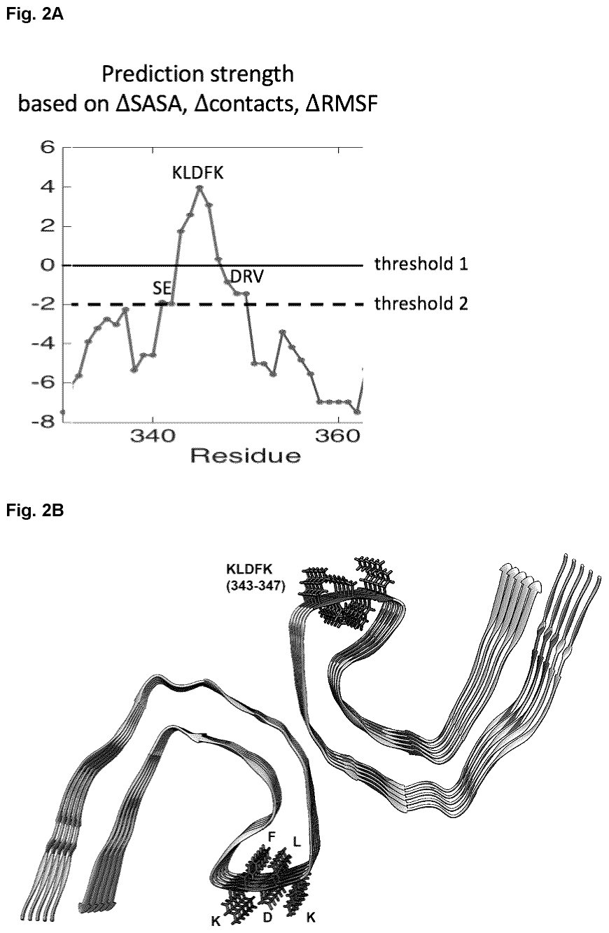

As indicated above, sequences within residues 337 to 352 of tau were identified to be preferentially exposed under biasing conditions, which corresponds to the VEVKSEKLDFKDRVQS (SEQ ID NO: 23). Additional epitopes are provided by SEQ ID NO: 23, including for example any 4 or greater amino acid stretch, EKLDFKDR (SEQ ID NO: 24), KLDFKDR (SEQ ID NO: 25), or SEKLDFKDRV (SEQ ID NO: 26). As shown in FIG. 2A, when ΔSASA, Δcontacts and ΔRMSF are considered together residues in the sequence KLDFK (SEQ ID NO: 1) have the highest prediction strength. Using a lower threshold, the epitope can also include SE or a part thereof on the N-terminus or DRV, or a part thereof on the C-terminus.

KLDF (SEQ ID NO: 2) is present at amino acids 343 to 346 of PDB 5O3L, LDFK (SEQ ID NO: 3) is present at amino acids 344 to 347 and KLDFK (SEQ ID NO: 1) at amino acids 343 to 347.

Sixteen different cyclic peptide sequences were generated by adding 1-4 glycines on either side of predicted epitope sequences (e.g. KLDFK (SEQ ID NO: 1), KLDF (SEQ ID NO: 2) and LDFK (SEQ ID NO: 3)), N-terminal and C-terminal. A cysteine residue was included to tether the construct to a protein (e.g. KLH or BSA). Possible cyclic peptide sequences include but are not limited to cyclo(CGKLDFKG) (SEQ ID NO: 4), cyclo(CGKLDFKGG) (SEQ ID NO: 5), and cyclo(CGGKLDFKG) (SEQ ID NO: 6), etc. MD simulations were run for 300 ns (600 ns for KLDF (SEQ ID NO: 2) scaffolds) on each of these 16 sequences to generate either 2500 snapshot conformations (KLDFK (SEQ ID NO: 1) or LDFK (SEQ ID NO: 3)) or 6000 cyclic peptide snapshot conformations (KLDF (SEQ ID NO: 2)).

The different cyclic compounds comprising the epitopes in an amino acid scaffold (e.g. comprising a linker) were assessed for their suitability for presenting the epitope as described herein and in Example 2 and used for further analysis.

The dissimilarity between the epitope conformation in cyclic peptide ensemble and its conformation in either the fibril ensemble or the monomer ensemble can be quantified by using the Jensen-Shannon distance (JSD). This distance gives an effective separation between any two pairs of ensembles, which may be recast as an effective separation between two Gaussian ensembles. Cyclic peptide scaffolds of the epitopes KLDF (SEQ ID NO: 2) (e.g. cyclo(CGGGGKLDFG) (SEQ ID NO: 19)), and likewise LDFK (SEQ ID NO: 3) and KLDFK (SEQ ID NO: 1), whose ensembles are distinct from that of the tau monomer and scaffolds that are also similar to the biased or stressed fibril are desired. These two criteria, large JSD to the monomer ensemble and small JSD to the stressed fibril ensemble, were used to assess different scaffolds.

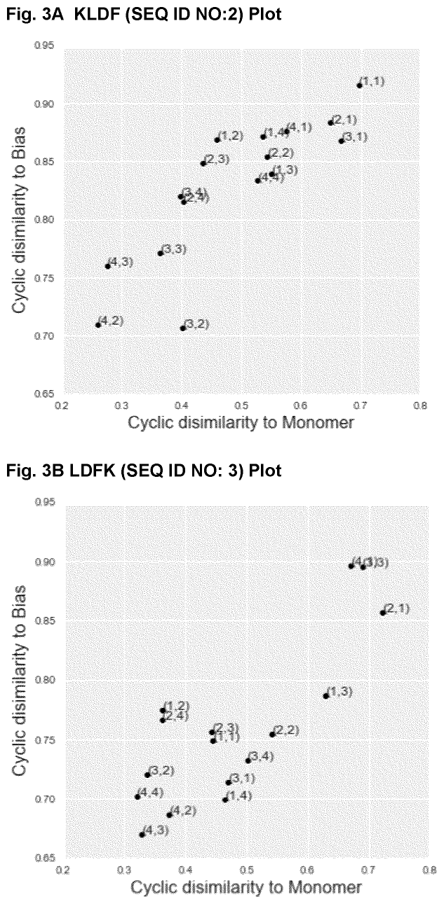

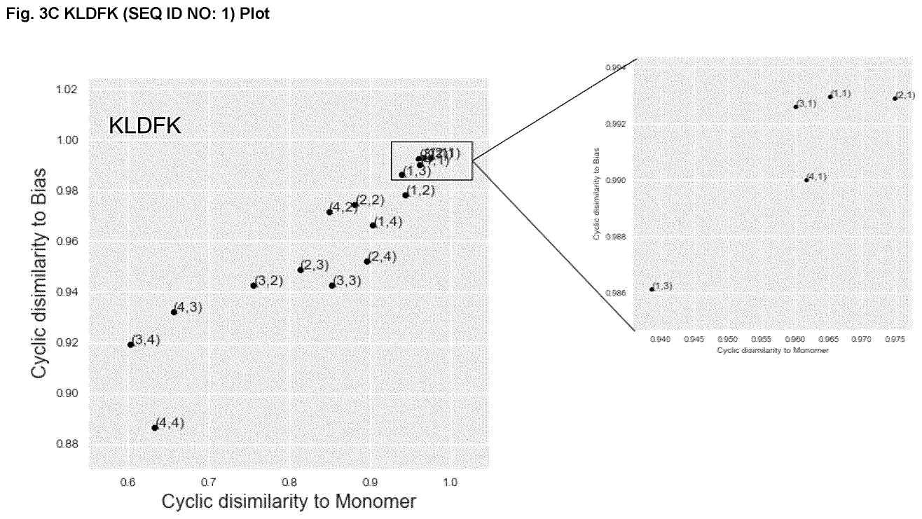

FIGS. 3A, B and C show scatter plots of the JSD for 16 cyclic peptide scaffolds for epitopes KLDF (SEQ ID NO: 2), LDFK (SEQ ID NO: 3), and KLDFK (SEQ ID NO: 1). A JSD of XX corresponds to an effective distance of 7.8 standard deviations (XX σ) between two one-dimensional gaussians.

### Example 2

Scaffolding that can be used to present the identified epitopes in a cyclic conformation were assessed.

Table 2 below gives several cyclic epitope scaffolds for KLDF (SEQ ID NO: 2), obtained by flanking the epitope with a variable number of glycine amino acids N- and C-terminal to the epitope and a cysteine residue. Suitability is assessed by measuring the Jenson-Shannon-distance (JSD) between the ensembles of the cyclic peptide and the equilibrium ensembles of the tau monomer, and, between the ensembles of the cyclic peptide and the equilibrium ensemble of the stressed (i.e. biased) fibril. Similarity to the stressed/biased fibril is desired, while dissimilarity to the monomer ensembles is also desired to avoid interference with in vivo function. Cyclic peptide scaffolds that are predicted to be suitable based on these criteria are described in Table 2.

FIG. 3A shows a scatter plot of this data for the 16 cyclic epitope scaffolds for KLDF (SEQ ID NO: 2).

TABLE 2
Cyclic peptides for epitope KLDF
(SEQ ID NO: 2)

| | SEQ |
|---|---|
| | ID |
| Cyclic peptide | NO: |
| CGKLDFG (1, 1) | 7 |
| CGKLDFGG (1, 2) | 8 |
| CGKLDFGGG (1, 3) | 9 |
| CGKLDFGGGG (1, 4) | 10 |
| CGGKLDFG (2, 1) | 11 |
| CGGKLDFGG (2, 2) | 12 |
| CGGKLDFGGG (2, 3) | 13 |
| CGGKLDFGGGG (2, 4) | 14 |
| CGGGKLDFG (3, 1) | 15 |
| CGGGKLDFGG (3, 2) | 16 |
| CGGGKLDFGGG (3, 3) | 17 |
| CGGGKLDFGGGG (3, 4) | 18 |
| CGGGGKLDFG (4, 1) | 19 |
| CGGGGKLDFGG (4, 2) | 20 |
| CGGGGKLDFGGG (4, 3) | 21 |
| CGGGGKLDFGGGG (4, 4) | 22 |

TABLE 3
Similarity of cyclic contructs to biased,
fibril and monomer ensemles as measured
by JSD

| | cyclic- | cyclic- |
|---|---|---|
| | bias: | monomer: |
| Cyclic construct | (1-JSD) | JSD |
| CGKLDFG (1, 1) | 0.12 | 0.61 |
| CGKLDFGG (1, 2) | 0.13 | 0.40 |
| CGKLDFGGG (1, 3) | 0.18 | 0.51 |
| CGKLDFGGGG (1, 4) | 0.21 | 0.34 |
| CGGKLDFG (2, 1) | 0.19 | 0.39 |
| CGGKLDFGG (2, 2) | 0.18 | 0.37 |
| CGGKLDFGGG (2, 3) | 0.14 | 0.45 |
| CGGKLDFGGGG (2, 4) | 0.27 | 0.28 |
| CGGGKLDFG (3, 1) | 0.10 | 0.78 |
| CGGGKLDFGG (3, 2) | 0.27 | 0.43 |
| CGGGKLDFGGG (3, 3) | 0.23 | 0.25 |
| CGGGKLDFGGGG (3, 4) | 0.19 | 0.42 |
| CGGGGKLDFG (4, 1) | 0.11 | 0.68 |
| CGGGGKLDFGG (4, 2) | 0.28 | 0.22 |
| CGGGGKLDFGGG (4, 3) | 0.17 | 0.37 |
| CGGGGKLDFGGGG (4, 4) | 0.22 | 0.34 |

A similar analysis was conducted for epitope KLDFK (SEQ ID NO: 1) and LDFK (SEQ ID NO: 3). Suitable scaffolds are provided in Table 4.

TABLE 4
Cyclic peptides for epitope KLDFK
(SEQ ID NO: 1) and LDFK (SEQ ID NO: 3)

| | SEQ | | SEQ |
|---|---|---|---|
| | ID | | ID |
| Cyclic peptide | NO: | Cyclic peptide | NO: |
| CGKLDFKG | 27 | CGLDFKG | 48 |
| CGKLDFKGG | 28 | CGLDFKGG | 49 |
| CGKLDFKGGG | 29 | CGLDFKGGG | 50 |
| CGKLDFKGGGG | 30 | CGLDFKGGGG | 51 |
| CGGKLDFKG | 31 | CGGLDFKG | 52 |
| CGGKLDFKGG | 32 | CGGLDFKGG | 53 |
| CGGKLDFKGGG | 33 | CGGLDFKGGG | 54 |
| CGGKLDFKGGGG | 34 | CGGLDFKGGGG | 55 |
| CGGGKLDFKG | 35 | CGGGLDFKG | 56 |
| CGGGKLDFKGG | 36 | CGGGLDFKGG | 57 |
| CGGGKLDFKGGG | 37 | CGGGLDFKGGG | 58 |
| CGGGKLDFKGGGG | 38 | CGGGLDFKGGGG | 59 |
| CGGGGKLDFKG | 39 | CGGGGLDFKG | 60 |
| CGGGGKLDFKGG | 40 | CGGGGLDFKGG | 61 |
| CGGGGKLDFKGGG | 41 | CGGGGLDFKGGG | 62 |
| CGGGGKLDFKGGGG | 42 | CGGGGLDFKGGGG | 63 |

FIGS. 3B and 30 shows a scatter plot of this data for the 16 cyclic epitope scaffolds for LDFK (SEQ ID NO: 3) and KLDFK (SEQ ID NO: 1) respectively.

TABLE 5
Similarity of cyclic contructs to biased
fibril and monomer ensemles as measured
by JSD

| | cyclic- | 1-cyclic- | |
|---|---|---|---|
| Cyclic | monomer- | bias- | SEQ ID |
| construct | JSD | JSD | NO: |
| CGKLDFKG | 0.53 | 0.15 | 27 |
| CGKLDFKGG | 0.95 | 0.019 | 28 |
| CGKLDFKGGG | 0.93 | 0.016 | 29 |
| CGKLDFKGGGG | 0.85 | 0.046 | 30 |
| CGGKLDFKG | 0.95 | 0.017 | 31 |
| CGGKLDFKGG | 0.89 | 0.022 | 32 |
| CGGKLDFKGGG | 0.74 | 0.057 | 33 |
| CGGKLDFKGGGG | 0.86 | 0.061 | 34 |
| CGGGKLDFKG | 0.92 | 0.024 | 35 |
| CGGGKLDFKGG | 0.74 | 0.062 | 36 |
| CGGGKLDFKGGG | 0.79 | 0.081 | 37 |
| CGGGKLDFKGGGG | 0.62 | 0.083 | 38 |
| CGGGGKLDFKG | 0.95 | 0.015 | 39 |
| CGGGGKLDFKGG | 0.81 | 0.03 | 40 |
| CGGGGKLDFKGGG | 0.66 | 0.069 | 41 |
| CGGGGKLDFKGGGG | 0.66 | 0.11 | 42 |

TABLE 6
Similarity of cyclic contructs to biased fibril
and monomer ensemles as measured by JSD

| | cyclic- | | |
|---|---|---|---|
| | monomer- | 1-cyclic- | SEQ |
| Cyclic construct | JSD | bias-JSD | ID NO: |
| CGLDFKG | 0.44 | 0.25 | 48 |
| CGLDFKGG | 0.36 | 0.22 | 49 |
| CGLDFKGGG | 0.63 | 0.21 | 50 |
| CGLDFKGGGG | 0.46 | 0.30 | 51 |
| CGGLDFKG | 0.72 | 0.14 | 52 |
| CGGLDFKGG | 0.54 | 0.24 | 53 |
| CGGLDFKGGG | 0.44 | 0.24 | 54 |
| CGGLDFKGGGG | 0.36 | 0.23 | 55 |
| CGGGLDFKG | 0.47 | 0.29 | 56 |
| CGGGLDFKGG | 0.34 | 0.28 | 57 |
| CGGGLDFKGGG | 0.69 | 0.10 | 58 |
| CGGGLDFKGGGG | 0.50 | 0.27 | 59 |
| CGGGGLDFKG | 0.67 | 0.10 | 60 |
| CGGGGLDFKGG | 0.37 | 0.31 | 61 |
| CGGGGLDFKGGG | 0.33 | 0.33 | 62 |
| CGGGGLDFKGGGG | 0.32 | 0.30 | 63 |

### Example 3

Cyclic Compound Construction Comprising Predicted Epitopes

Compounds comprising the predicted epitope sequences can be prepared by making linear peptides comprising or consisting an epitope described herein such as KLDFK (SEQ ID NO: 1), KLDF (SEQ ID NO: 2), or LDFK (SEQ ID NO: 3) and a linker sequence and cyclized to make cyclic compounds such as Cyclo(CGKLDFKGG) (SEQ ID NO: 28) or cyclo(CGGKLDFKGGGG) (SEQ ID NO:34). For example, the cyclic compounds can be made by cyclizing linear peptides head to tail.

For example, a peptide comprising an epitope sequence such as KLDF (SEQ ID NO: 2), or LDFK (SEQ ID NO: 3) can be synthesized with or conjugated to a linker, preferably comprising 1, 2, 3, or 4 amino acids such as glycine and/or PEG units C terminal and/or N terminal to the epitope sequence. A cysteine residue or other functionalizable residue can be added as part of the linker as well. When the linker is composed of an amino acid sequence, it can be synthesized using known methods such as Fmoc based solid phase peptide synthesis alone or in combination with other methods. PEG molecules can be coupled to amine groups at the N terminus for example using coupling chemistries described in Hamley 2014 [Biomacromolecules, 2014, 15 (5), pp 1543-1559, DOI: 10.1021/bm500246w] and Roberts et al 2012 [Advanced Drug Delivery Reviews, Volume 64, Supplement, December 2012, Pages 116-127; M. J. Roberts M. D. Bentley J. M. Harris https://doi.org/10.1016/j.addr.2012.09.025], each incorporated herein by reference. The compounds may be cyclized by covalently bonding 1) the amino terminus and the carboxy terminus of the peptide+linker to form a peptide bond (e.g. cyclizing the backbone), 2) the amino or carboxy terminus with a side chain in the peptide+linker or 3) two side chains in the peptide+linker.

The bonds in the cyclic compound may be all regular peptide bonds (homodetic cyclic peptide) or include other types of bonds such as ester, ether, amide or disulfide linkages (heterodetic cyclic peptide).

Peptides may be cyclized by oxidation of thiol- or mercaptan-containing residues at the N-terminus or C-terminus, or internal to the peptide, including for example cysteine and homocysteine. For example, two cysteine residues flanking the peptide may be oxidized to form a disulphide bond. Oxidative reagents that may employed include, for example, oxygen (air), dimethyl sulphoxide, oxidized glutathione, cystine, copper (II) chloride, potassium ferricyanide, thallium(Ill) trifluro acetate, or other oxidative reagents such as may be known to those of skill in the art and used with such methods as are known to those of skill in the art.

Methods and compositions related to cyclic peptide synthesis are described in US Patent Publication 2009/0215172. US Patent publication 2010/0240865, US Patent Publication 2010/0137559, and U.S. Pat. No. 7,569,541 describe various methods for cyclization. Other examples are described in PCT Publication WO01/92466, and Andreu et al., 1994. Methods in Molecular Biology 35:91-169. Each of these references are hereby incorporated herein by reference in their entirety.

As mentioned, the linker can comprise one or more cysteine residues flanking and/or inserted in the linker. The peptide can be structured into a cyclic conformation by creating a disulfide linkage between the non-native cysteines residues added to the N- and C-termini of the peptide.

The cyclic peptide can be linked to a carrier, optionally a BSA moiety or an immunogenicity enhancing agent such as KLH.

Linear and cyclic peptides comprising an epitope sequence described herein can be prepared. Examples are provided in Table 7.

TABLE 7
Cyclic peptides for immunogens and corresponding
linear peptide

| | | |
|---|---|---|
| Linear CGGKLDFKGGGG | (SEQ ID NO: 34) | (2, 4 linker) |
| Cyclo(CGKLDFKGGGG) | (SEQ ID NO: 34) | (2, 4 linker) |
| Linear CGKLDFKGG | (SEQ ID NO: 28) | (1, 2 linker) |
| Cyclo(CGKLDFKGG) | (SEQ ID NO: 28) | (1, 2 linker) |
| Linear CGGKLDFKG | (SEQ ID NO: 31) | (2, 1 linker) |
| Cyclo(CGGKLDFKG) | (SEQ ID NO: 31) | (2, 1 linker) |
| Linear CGGGKLDFGG | (SEQ ID NO: 16) | (3, 2 linker) |
| Cyclo(CGGGKLDFGG) | (SEQ ID NO: 16) | (3, 2 linker) |
| Linear CGGGGKLDFG | (SEQ ID NO: 19) | (4, 1 linker) |
| Cyclo(CGGGGKLDFG) | (SEQ ID NO: 19) | (4, 1 linker) |
| Linear CGGGKLDFG | (SEQ ID NO: 15) | (3, 1 linker) |
| Cyclo(CGGGKLDFG) | (SEQ ID NO: 15) | (3, 1 linker) |

Peptide synthesis is performed by CPC Scientific Inc. (Sunnyvale Calif., USA). The peptides are synthesized by standard conventional Fmoc-based solid-phase peptide synthesis on 2-chlorotrityl chloride resin, followed by cleavage from the resin. Peptide sequence is confirmed by electrospray MS and purity assessed by HPLC to confirm at least 95% pure. Cyclization can be performed via a head-to-tail (C-G) amide bond. Non-cyclized, linear peptide is also produced by CPC Scientific.

Immunogen Construction

The cyclic compounds can then be conjugated to KLH (for immunizing) or BSA (for screening) for example via maleimide-based coupling (CPC Scientific Inc, Sunnyvale Calif.).

### Example 4

Cyclic peptides (4,1) cyclo(CGGGGKLDFG) (SEQ ID NO: 19), (3,2) cyclo(CGGGKLDFGG) (SEQ ID NO: 16) and (3,1) cyclo(CGGGKLDFG) (SEQ ID NO: 15) were prepared by and conjugated to KLH or BSA by CPC Scientific Inc. (Sunnyvale Calif., USA) as described in Example 3 and used to generate antibodies as described in Example 5.

### Example 5

Antibody Generation and Selection

The linked peptides were used for mouse monoclonal antibody production, following protocols approved by the Canadian Council on Animal Care (Immunoprecise Antibodies LTD (Victoria BC, Canada), referred to herein as IPA).

Immunization

Briefly, female BALB/c mice (Charles River Laboratories, Quebec) were immunized with KLH-conjugated cyclic peptides using IPA's Rapid Prime Immunization procedure. Mice were housed in a ventilated rack system from Lab Products. All mice were euthanized on Day 19 and lymphocytes were harvested for hybridoma cell line generation.

Fusion/Hybridoma Development

Lymphocytes were isolated and fused with murine SP2/0 myeloma cells in the presence of poly-ethylene glycol (PEG 1500) or via electrofusion. Fused cells were cultured using HAT selection. This method uses a semi-solid methylcellulose-based HAT selective medium to combine the hybridoma selection and cloning into one step. Single cell-derived hybridomas are grown to form monoclonal colonies on the semi-solid media. About 10 days after the fusion event, resulting hybridoma clones can be transferred to 96-well tissue culture plates and grown in HT containing medium until mid-log growth is reached (5 days).

Hybridoma Analysis

Tissue culture supernatants from the hybridomas were tested by indirect ELISA on screening antigen (cyclic peptide-BSA and linear peptide-BSA) and were probed for both IgG and IgM antibodies using a Goat anti-IgG/IgM(H&L)-HRP secondary antibody and developed with TMB substrate.

Positive cultures were retested on screening antigen to confirm secretion and on an irrelevant antigen (such as Human Transferrin). Clones were isotyped by antibody trapping ELISA to determine if they were IgG or IgM isotype and tested by indirect ELISA on other cyclic peptide-BSA conjugates comprising the same epitope to evaluate cross-reactivity.

Isotypinq

The hybridoma antibodies can be isotyped using antibody trap experiments. Trap plates were coated with 1:10,000 Goat anti-mouse IgG/IgM(H&L) antibody at 100 uL/well carbonate coating buffer pH9.6 overnight at 4 C. Primary antibody (hybridoma supernatants) is added at 100 ug/mL. Secondary Antibody is added at 1:5,000 Goat anti-mouse IgGγ-HRP or 1:10,000 Goat anti-mouse IgMμ-HRP at 100 uL/well in PBS-Tween for 1 hour at 37 C with shaking. All washing steps are performed for 30 mins with PBS-Tween. The substrate TMB is added at 50 uL/well, developed in the dark and stopped with equal volume 1 M HCl.

### Results

Antibodies obtained by immunizing with (4,1) cyclo(CGGGGKLDFG) (SEQ ID NO: 19), (3,2) cyclo(CGGGKLDFGG) (SEQ ID NO: 16) and (3,1) cyclo(CGGGKLDFG) (SEQ ID NO: 15) selectively bound the cyclic peptide relative to the corresponding linear peptide by ELISA. As shown in Table 8 below, the majority of the antibodies analyzed were reactive with the cyclic peptide and showed little or low reactivity with the corresponding linear peptide under the conditions tested. Most antibodies reacted with the cyclic peptides of one or both of the other cyclic peptide. A subset of clones reacted only with the cyclic peptide to which it was raised. (OD=optical density)

TABLE 8
Reactivity of antibody clones to cyclic and linear peptides

| | | | |
|---|---|---|---|
| | Cyclic | Linear | Cross-react with other |
| Antibody | (OD) | (OD) | cyclic peptide(s) |

KLDF (4,1)

| | | | |
|---|---|---|---|
| Ab1 | 1.932 | 0.1 | Y |
| Ab2 | 1.866 | 0.142 | N |
| Ab3 | 1.794 | 1.611 | Y |
| Ab4 | 1.932 | 0.07 | N |
| Ab5 | 1.923 | 0.243 | Y |
| Ab6 | 1.801 | 0.119 | Y |
| Ab7 | 1.896 | 0.896 | Y |
| Ab8 | 1.935 | 0.075 | Y |
| Ab9 | 1.745 | 0.046 | Y |

KLDF (3,2)

| | | | |
|---|---|---|---|
| Ab10 | 1.598 | 0.78 | Y |
| Ab11 | 1.644 | 0.232 | Y |
| Ab12 | 1.744 | 0.784 | Y |
| Ab13 | 1.293 | 0.055 | Y |
| Ab14 | 1.701 | 0.788 | Y |
| Ab15 | 1.579 | 0.774 | Y |
| Ab16 | 1.578 | 0.916 | Y |
| Ab17 | 1.629 | 0.302 | Y |
| Ab18 | 1.63 | 0.501 | Y |
| Ab19 | 1.393 | 0.047 | Y |

KLDF (3,1)

| | | | |
|---|---|---|---|
| Ab20 | 1.236 | 1.849 | Y |
| Ab21 | 1.213 | 0.107 | Y |
| Ab22 | 1.524 | 1.978 | Y |
| Ab23 | 1.19 | 1.691 | Y |
| Ab24 | 1.399 | 1.689 | Y |
| Ab25 | 1.244 | 1.656 | Y |
| Ab26 | 1.456 | 0.06 | Y |
| Ab27 | 1.486 | 0.077 | N |
| Ab28 | 1.481 | 0.054 | N |
| Ab29 | 1.353 | 0.075 | N |

### Example 6

Anti-Misfolded Oligomeric Tau Antibody Characterization

Antibodies were tested for their ability to bind monomeric tau polypeptide as well as misfolded oligomeric tau polypeptide using surface plasmon resonance. Surface plasmon resonance measurements were performed using a Molecular Affinity Screening System (Sierra Sensors, Hamburg, Germany). Tau monomers (Stressmarq, Victoria, BC, Canada) or tau oligomers (SynAging, Vandoeuvre-les-Nancy, France) were immobilized on high amine capacity sensorchips and hybridoma supernatants (50% dilution) were injected over the immobilized surfaces at 10 μl/minute for 4 min followed by a 5 min dissociation phase. Mouse IgG1 was used as a negative control and a pan-tau reactive antibody was used as a positive control. The reverse orientation was used for purified antibodies: the mAbs were immobilized covalently on the surface of sensorchips (8000-12000 RUs) and serial dilutions of tau monomers or oligomers were injected over the surface.

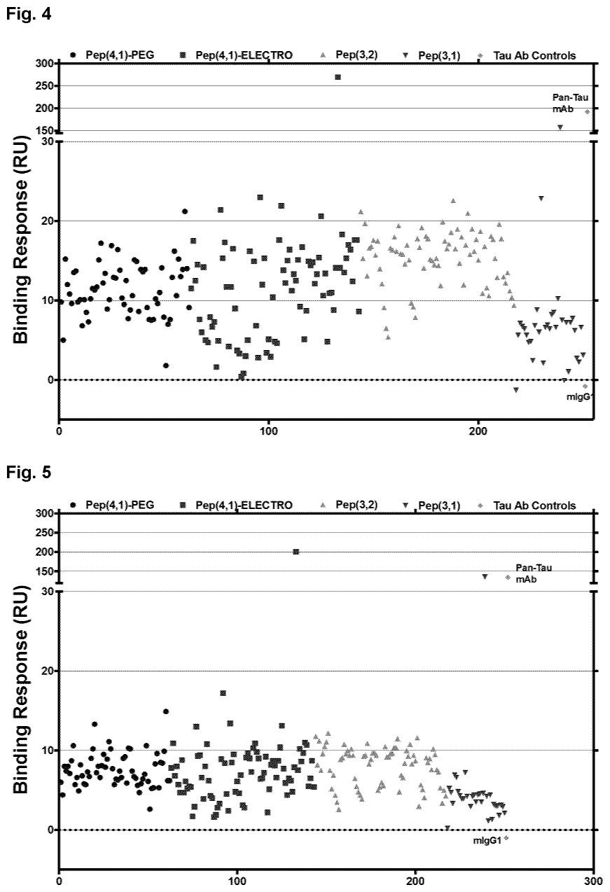

FIG. 4 shows the binding response (RU-response units) of hybridoma clone supernatants to immobilized tau oligomers.

FIG. 5 shows the binding response (RU-response units) of hybridoma clone supernatants to immobilized tau monomers.

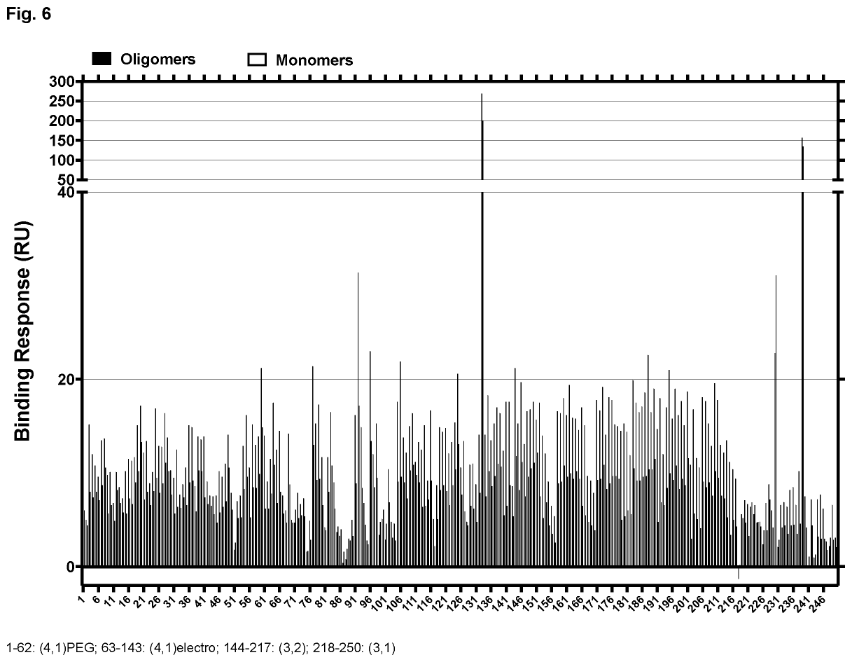

FIG. 6 superimposes binding responses to tau oligomers and monomers for each hybridoma clone.

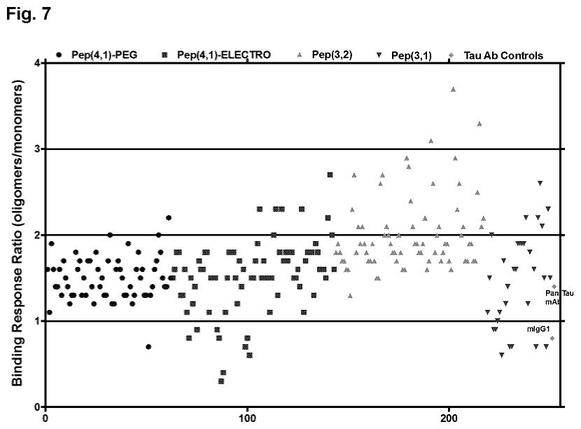

FIG. 7 shows the ratio of the binding response to tau oligomers/tau monomers for each hybridoma clone. The results indicate that all 3 cyclic peptide scaffolds gave rise to multiple antibody clones that preferentially bind tau oligomers vs monomers (ratio of oligomer to monomer binding greater than the 1.4 ratio obtained with the non-selective pan-tau antibody).

Table 9 below lists 29 hybridoma clone supernatants and shows their binding response to tau oligomers and monomers for each hybridoma clone.

TABLE 9
Hybridoma supernatant binding response to tau oligomers and monomers

| | | | | |
|---|---|---|---|---|
| | | Oligomers | Monomers | Ratio |
| Antibody | Clone | [RU] | [RU] | (O/M) |

KLDF (4,1)

| | | | | |
|---|---|---|---|---|
| Ab1 | 1E10 | 31.4 | 17.2 | 1.8 |
| Ab2 | 1G2 | 21.9 | 9.6 | 2.3 |
| Ab3 | 2C7 | 269.4 | 200.8 | 1.3 |
| Ab4 | 3A12 | 17.3 | 9.4 | 1.8 |
| Ab5 | 3H8 | 14.1 | 7.5 | 1.9 |
| Ab6 | 7H5 | 15.1 | 6.5 | 2.3 |
| Ab7 | 8A11 | 13.4 | 5.9 | 2.3 |
| Ab8 | 8B9 | 17.6 | 6.5 | 2.7 |
| Ab9 | 8F8 | 16.7 | 9.2 | 1.8 |

KLDF (3,2)

| | | | | |
|---|---|---|---|---|
| Ab10 | 9D12 | 14.0 | 5.2 | 2.7 |
| Ab11 | 9E4 | 17.0 | 6.5 | 2.6 |
| Ab12 | 10B10 | 14.5 | 5.0 | 2.9 |
| Ab13 | 10C9 | 14.7 | 4.8 | 3.1 |
| Ab14 | 10D4 | 18.0 | 6.9 | 2.6 |
| Ab15 | 10D9 | 10.9 | 3.0 | 3.7 |
| Ab16 | 10F2 | 16.8 | 5.7 | 2.9 |
| Ab17 | 10F3 | 13.5 | 5.3 | 2.5 |
| Ab18 | 10F10 | 11.2 | 3.4 | 3.3 |
| Ab19 | 12A10 | 10.6 | 4.1 | 2.6 |

KLDF (3,1)

| | | | | |
|---|---|---|---|---|
| Ab20 | 2A9 | 22.8 | 31.1 | 0.7 |
| Ab21 | 2C6 | 6.6 | 3.5 | 1.9 |
| Ab22 | 5F10 | 6.7 | 3.3 | 2.0 |
| Ab23 | 8E11 | 10.2 | 4.6 | 2.2 |
| Ab24 | 8G7 | 156.9 | 135.4 | 1.2 |
| Ab25 | 9B6 | 7.2 | 3.2 | 2.2 |
| Ab26 | 9C6 | 7.7 | 3.0 | 2.6 |
| Ab27 | 9E4 | 6.6 | 2.9 | 2.3 |
| Ab28 | 11F8 | 6.4 | 3.5 | 1.9 |
| Ab29 | 12D11 | 6.2 | 3.0 | 2.1 |

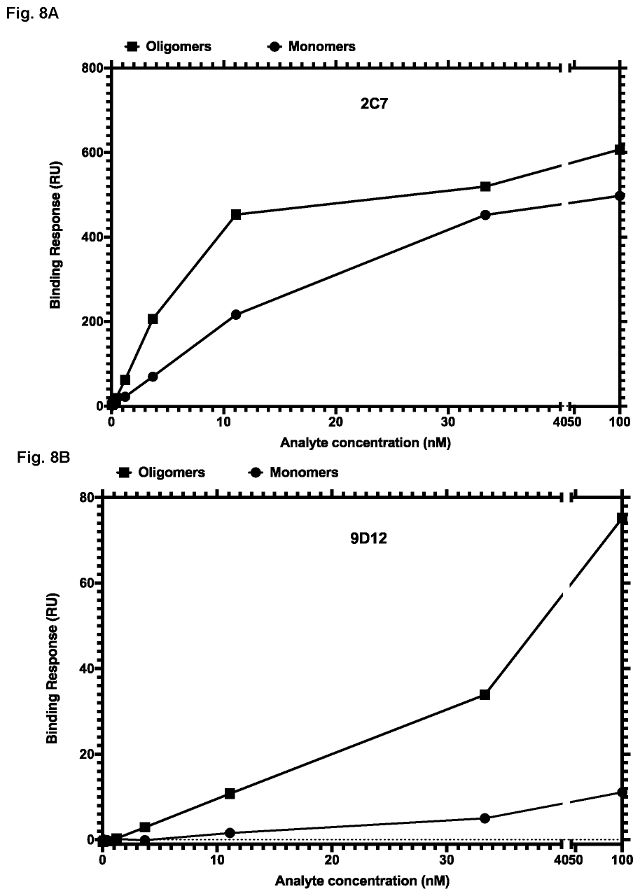

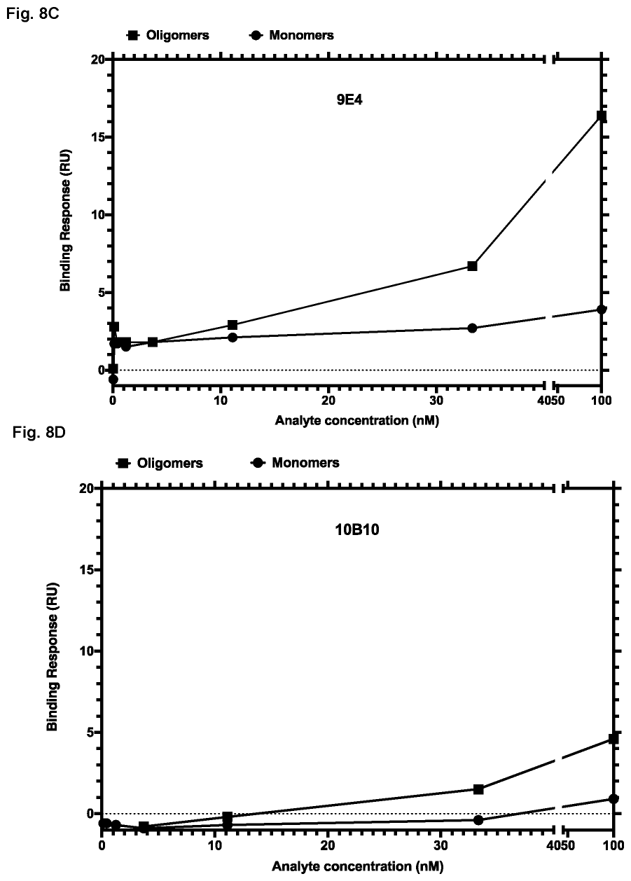

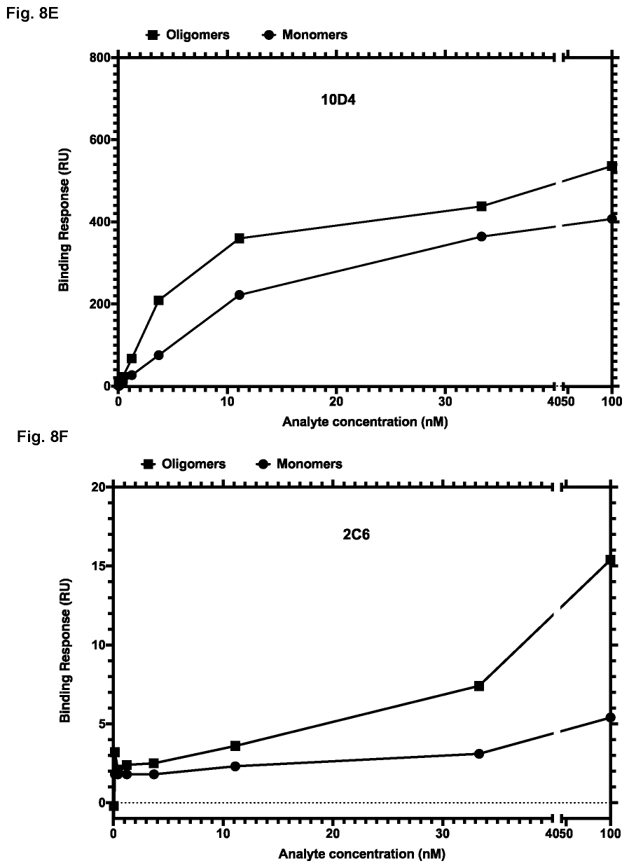

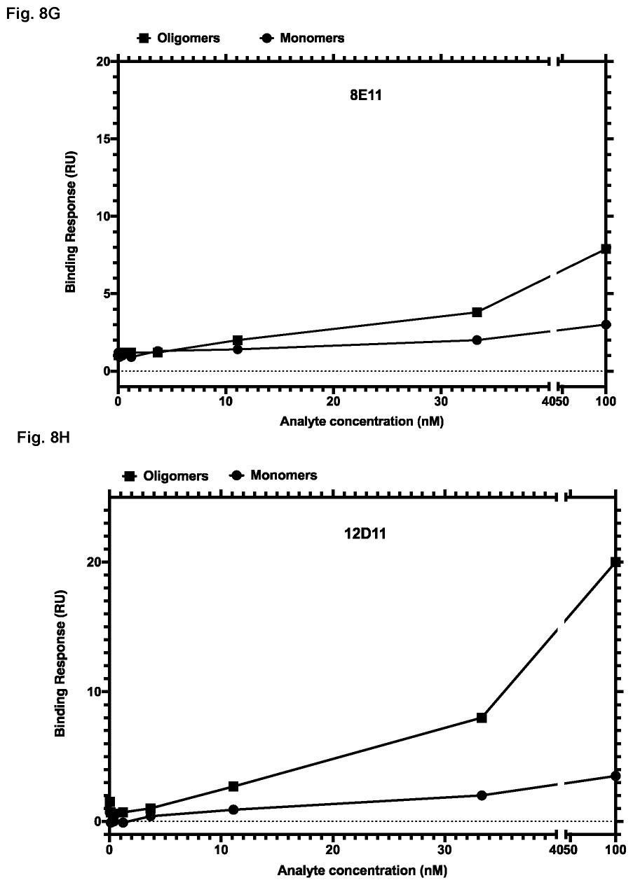

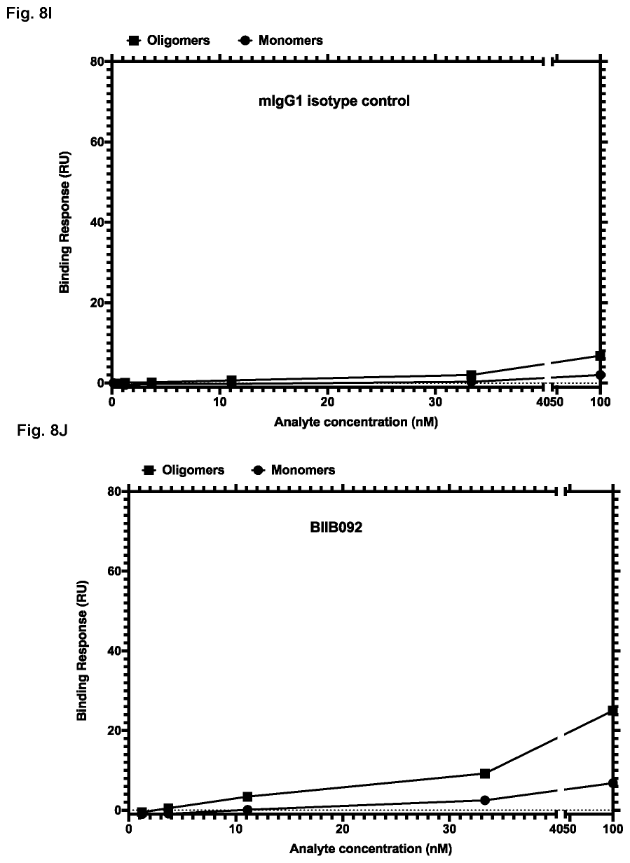

FIGS. 8A-H shows the binding response of immobilized purified mAbs (approximately 8,000-12,000 RUs on sensor chip) to varying concentrations of tau monomers or oligomers injected over the surface. FIG. 81 shows IgG control and FIG. 8J shows another anti-tau antibody, Gosuranemab or BIIB092. Binding responses (RU) measured at 30s post-injection stop in the dissociation phase are shown.

### Example 7

Surface Plasmon Resonance Analysis of Biological Samples

Homogenization: Human neurological tissue samples were submersed in a volume of fresh, ice cold TBS (supplemented with 5 mM EGTA, 5 mM EDTA, (both from Sigma) and EDTA-free protease inhibitor cocktail from Roche Diagnostics, Laval QC, Canada) such that the final concentration of tissue was 10% (w/v). Tissue was homogenized in this buffer using a mechanical probe homogenizer (3×30 sec pulses with 30 sec pauses in between, all performed on ice). TBS homogenized samples were then subjected to ultracentrifugation (100,000×g for 90 min). Supernatants were collected, aliquoted and stored at −80° C. The protein concentration of TBS homogenates was determined using a BCA protein assay (Pierce Biotechnology Inc, Rockford Ill., USA).

Surface Plasmon Resonance Analysis: Neurological tissue samples from AD patients were analyzed. Test antibodies, positive control antibody and IgG isotype control were immobilized at high densities (˜10,000 RU) (Approximately 8,000 to 12,000 RUs) on flow cells of a sensor chip. Diluted samples (200 pg protein/ml) were injected sequentially over the surfaces for approximately 300-900 seconds, followed by 150 seconds of dissociation in buffer and surface regeneration.

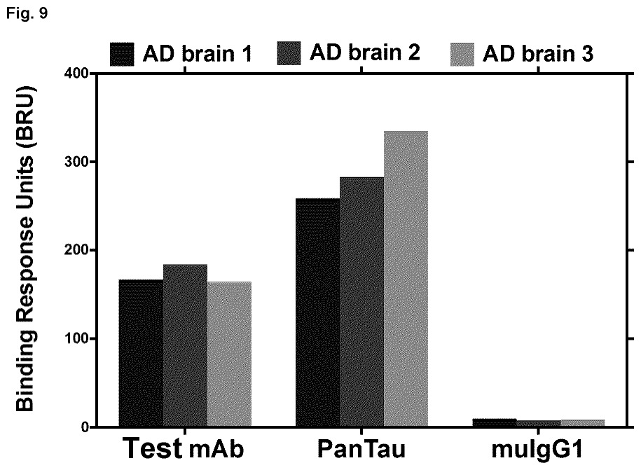

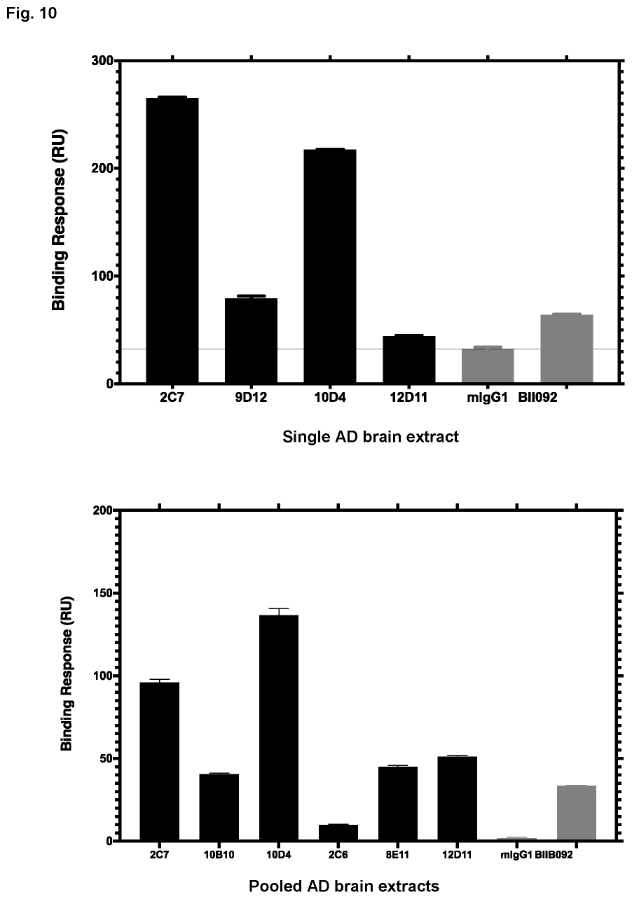

Results: Representative binding responses to extract from 3 individual AD brains for clone 8G7 (test mAb), a commercial pan tau antibody (positive control) and murine IgG1 (negative control) are shown in FIG. 9. FIG. 10 shows binding responses of selected mAbs to soluble extract from an individual AD brain (panel A) or to a pool of soluble extract from 3 AD brains (panel B). Binding responses (BRU) measured at 30s post-injection stop in the dissociation phase are shown.

### Example 8

### Immunohistochemistry (IHC) Staining and Immunofluorescence of AD Brain

Frozen sections from the brain frontal cortex of a patient with AD are exposed to test antibody or control antibodies at a concentration of 4-10 pg/ml. Bound antibody is detected by the addition of horseradish peroxidase-conjugated sheep anti-mouse IgG (ECL, 1:1000 dilution) or rabbit anti-human IgG (Abcam, 1:5000 dilution). Diaminobezidine (DAB) chromogen reagent, the HRP enzyme substrate (Vector Laboratories), is then added to the sections to produce a brown color. The sections are counterstained with hematoxylin to visualize the cells and cell nuclei (bluish purple staining). For immunofluorescence, detection of bound antibody can be performed using Alexa fluor 568-conjugated goat anti-mouse IgG (Invitrogen) at a 1:1000 working concentration with DAPI counterstaining.

### Example 9

### Replication of Seeding Activity of Pre-Formed Fibrils Using Tau Peptides and Inhibition of Proteopathic Tau Seeding by Antibodies

### Surface Plasmon Resonance (SPR) Binding to Soluble Tau Fibrils

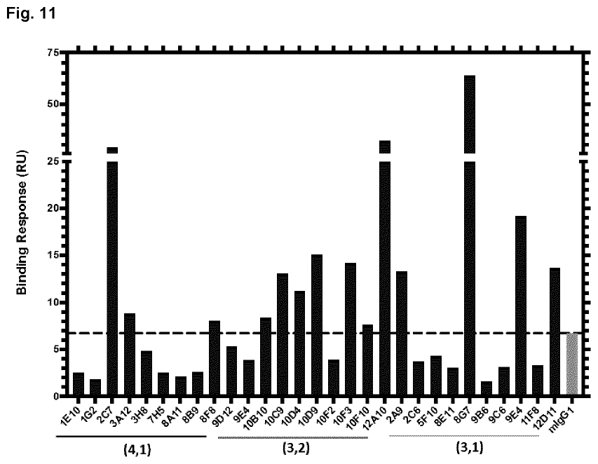

Selected antibody clones with hybridoma supernatants showing greater binding to tau oligomers vs monomers (see FIG. 6) were purified and assayed for binding to soluble tau fibrils (StressMarq Biosciences) by SPR. Soluble tau fibrils were immobilized on flow cells of a sensor chip (approximately 1200 RUs) and antibodies (1 uM) were injected over the surfaces for approximately 3 min followed by a dissociation period of approximately 5 min. A number of the antibodies cross-reacted with the soluble tau fibrils, as shown in FIG. 11.

### Inhibition of Ability of Soluble Pre-Formed Tau Fibrils (PFF) to Induce Intracellular Tau Aggregates

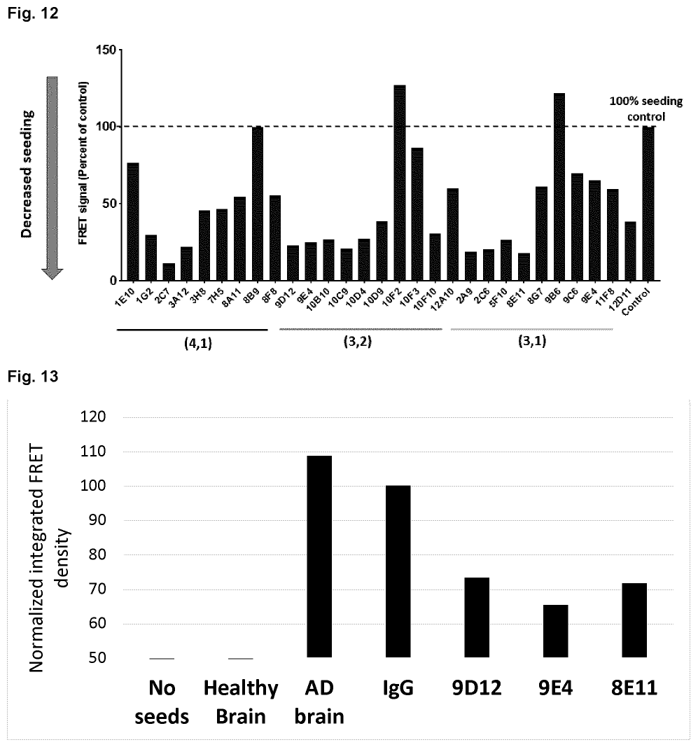

The ability of antibodies to inhibit proteopathic seeding by tau PFFs was tested as described by Holmes, B B et al., Proteopathic tau seeding predicts tauopathy in vivo. Proceedings of the National Academy of Sciences of the United States of America, 111(41): E4376-85, 2014 (hereby incorporated herein by reference in its entirety) using a cellular Fluorescence Energy Resonance Transfer (FRET) assay. Briefly, Tau RD P301S FRET Biosensor cells (ATCC) were exposed to tau PFFs (Tau441 (2N4R) P301S mutant from StressMarq) and lipofectamine to induce seeding. The Biosensor cells stably express tau protein fused to cyan fluorescent protein (CFP) as well as tau protein fused to yellow fluorescent protein (YFP). When aggregation occurs, the proximity of these 2 fluorescent labels gives rise to light emission at a different wavelength (526 nm vs 476 nm for the separate proteins) and the signal can be detected by flow cytometry. Biosensor cells were exposed to 0.6 ug/ml PFFs+lipofectamine and test antibodies (400 nM) were added approximately 20 min later. FRET detection by flow cytometry was performed 48 hours later. Results are shown in FIG. 12 and are expressed as a percentage of the FRET signal for cells cultured without antibody (100% seeding control). In this assay, the majority of tau antibodies inhibited seeding, resulting in lower levels of intracellular tau aggregates detected by FRET. As reported by Holmes, B B et al, proteopathic tau seeding activity in this assay is an “early and robust marker of tauopathy” in a mouse model. Inhibition of seeding by an antibody, as observed herein, would therefore be expected to inhibit tau pathogenesis.

### Example 10

### Inhibition of Seeding Activity of AD Brain Extract

mAbs Inhibit Induction of Aggregation by AD Brain Seeds

The ability of tau mAbs to inhibit the seeding activity of AD brain homogenate was assessed in a FRET assay using Tau RD P301S FRET Biosensor cells. Brain homogenate (20,000×g supernatant from 10% wt/vol homogenized brain tissue) was transduced into Biosensor cells using Lipofectamine 2000 reagent and FRET signal was measured 48 hr later by flow cytometry.

In studies testing direct inhibition of AD brain seeds by antibodies, brain homogenate+/−mAb (0.8 μM) was transduced into Biosensor cells. Results are expressed as Normalized Integrated FRET density (defined as the percent of FRET positive cells multiplied by the Median Fluorescence Intensity of those FRET positive cells and normalized to cells treated with IgG) and shown in FIG. 13. As expected, healthy brain homogenate was devoid of seeding activity while AD brain induced tau aggregation producing a FRET signal. The 3 antibodies tested (9D12, 9E4, 8E11) inhibited the seeding activity of AD brain homogenate compared to an IgG isotype control.

Pre-Treatment of AD Brain Extract with mAbs Reduces Seeding Activity

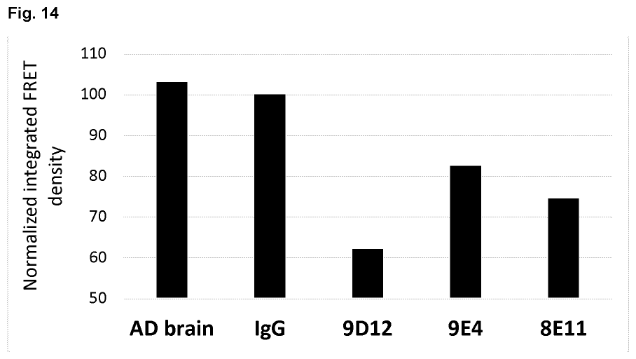

In immunodepletion studies testing the ability of antibodies to bind and deplete AD brain seeds, brain homogenate was pre-treated with mAb-coated magnetic beads (0.75 mg of Dynabeads Protein G incubated with 6 pg of each tau antibody or control IgG) for 30 min and the material remaining after removal of the beads and bound tau species was transduced into Biosensor cells. Results are expressed as Normalized Integrated FRET density. As shown in FIG. 14, pre-exposure of AD brain homogenate to the 3 antibodies tested (9D12, 9E4, 8E11) reduced seeding activity compared to an IgG isotype control.

### Example 11

### Antibody Sequencing

Ten mAb clones were selected for sequencing. Sequencing was performed by Next Generation Sequencing (NGS). From each of the 10 hybridomas, RNA was extracted and made into cDNA. The variable regions of IgG, IgK, and IgL were amplified in a 5′ RACE strategy. Hybridoma variable region amplicons were sequenced by the Illumina MiSeq next generation sequencer. Only antibody sequences accounting for at least 5% of the reads of each hybridoma were considered. All hybridomas had only one dominant sequence detected about the 5% threshold. All hybridoma light chains were identified as kappa. The sequences of the antibodies and CDRs identified are summarized in tables 10 and 11 below.

TABLE 10
Sequences of selected antibody variable regions. CDR regions
are indicated with bold and underline.

| Clone | | SEQ ID |
|---|---|---|
| (chain) | Amino Acid Sequence | NO: |
| 2C6.1 | EVQLQQSGAELVKPGASVKLSCTASGFNIKDTHMHWVKQRPEQGL | 75 |
| (heavy) | EWIGKIDPSNGNTQYDPKFQGKATITADTSSNTAYLQLSSLTSEDTA | |
| | VYYCATGFAYVVGQGTLVTVSA | |
| 2C6.1 | DIQMTQSPASLSASVGETVTITCRASGNIHNYLAWYQQKQGKSPQLL | 76 |
| (light) | VYNAKTLADGVPSRFSGSGSGTQYSLKINSLQPEDFGSYYCQHFW | |
| | YTPWTFGGGTKLEIK | |
| 8E11.1 | QVQLQQSGAELVRPGSSVKISCKASGYAFSSYWMNWVKQRPGQG | 77 |
| (heavy) | LEWIGQIYPGDGDTNYNGKFKGKATLTADKSSSTAYMQLSSLTSED | |
| | SAVYLCASQIYDGYYTFTYWGQGTLVTVSA | |
| 8E11.1 | DIVMSQSPSSLAVSAGEKVTMSCKSSQSLLNSRTRKNYLAWYQQK | 78 |
| (light) | PGQSPKLLIYWASTRVSGVPDRFTGSGSGTDFTLTISSVQAEDLAVY | |
| | YCKQSYNLWTFGGGTKLEIK | |
| 12011.1 | QIQLVQSGPELKKPGETVKISCKASGYTFTNYGMNWVKQAPGKGLK | 79 |
| (heavy) | WMGWINTYSGEPTYVDDFKGRFAFSLETSASTAYLQINNLKNEDMA | |
| | TYFCARSPGAYYTLDYWGQGTSVTVSS | |
| 12011.1 | DIVMSQSPSSLAVSAGEKVTMSCKSSQSLLNNRTRKNYLAWYQQK | 80 |
| (light) | PGQSPKLLIYWASTRESGVPDRFTGSGSGTDFTLTISSVQAEDLAVY | |
| | YCKQSYNLYTFGGGTKLEIK | |
| 9012.1 | QIQLVQSGPELKKPGETVKISCKASGYTFTNYGMNWVKQAPGKGLK | 81 |
| (heavy) | WMGWINTYTGEPTYTDDFKGRFAFSLETSASTAYLQINNLKNEDTAT | |
| | YFCGRGIRDYYTMDYWGQGTSVTVSS | |
| 9012.1 | DIVMSQSPSSLAVSAGEKVTMSCKSSQSLLNNRTRKNYAWYQQK | 82 |
| (light) | PGQSPKLLIYWASTRESGVPDRFTGSGSGTDFTLTISSVQAEDLAVY | |
| | YCKQSYNLYTFGGGTKLEIK | |
| 9E4.1 | DVQVQESGPGLVKPSQSLSLTCTVTGYSITSDYAWTWIRQFPGNKL | 83 |
| (heavy) | EWMGYISYSGSTSYNPSLKSRLSITRDTSKNQFFLQLNSVTTEDTAT | |
| | YYCAAYYRYGLAYFAYWGQGTLVTVSA | |
| 9E4.1 | DVVMTQTPLTLSVTIGQPASISCKSSQSLLDSDGKTYLNWLLQRPG | 84 |
| (light) | QSPKRLIYLVSKLDSGVPDRFTGSGSGTDFTLKISRVEAEDLGVYYC | |
| | WQGTHFPQTFGGGTKLEIK | |
| 10B10.1 | QIQLVQSGPELKKPGETVKISCKASGYTFTNFGMNWVKQAPGKGLK | 85 |
| (heavy) | WMGWINTFTGEPTYVDDFKGRFAFSLETSATTAYLQINNLKNEDTAT | |
| | YFCARSPGRVYTLDYWGQGTSVTVSS | |
| 10B10.1 | DIVMSQSPSSLAVSAGEKVTMSCKSSQSLLNSRTRKNYLAWYQQK | 86 |
| (light) | PGQSPKLLIYWASTRESGVPDRFTGSGSGTDFTLTISSVQAEDLAVY | |
| | YCKQSYNLYTFGGGTKLEIK | |
| 1009.1 | EVQLQQSGTVLARPGASVKMSCKASGYRFTSYWMYWVKQRPGQG | 87 |
| (heavy) | LEWIGAIYPGNSDTYNQRFKGKATLTAVTSASTAYMELSSLANEDSA | |
| | VYFCTRPYFDSWGQGTTLTVSS | |
| 1009.1 | DVVMTQTPLTLSVTIGQPASISCKSSQSLLDSDGKTYLNWLLQRPG | 88 |
| (light) | QSPKRLIYLVSKLDSGVPDRFTGSGSGTDFTLKISRVEAEDLGVYYC | |
| | WQGTHFPQTFGGGTKLEIK | |
| 1004.1 | DVQLQESGPGLVKPSQSLSLTCTVTGFSITSDYAWNWIRQFPGNKL | 89 |
| (heavy) | EWMGFIRYSGNTRFNPSLKGRGSITRDTSKNQFFLQLNSVTTEDTAT | |
| | YYCASTLEDSYWYFDVWGAGTTVTVSS | |
| 1004.1 | DVLMTQTPLSLPVSLGDQASISCRSSQSIVHTNGNTYLEWYLQKPG | 90 |
| (light) | QSPKLLIYKVSNRFSGVPDRFSGSGSGTDFTLKISRVEAEDLGVYYC | |
| | FQGSHVPLTFGAGTKLELK | |
| 1009.1 | QVQLQQSGAELVKPGASVKLSCKASGYTFTSYYMFWVKQRPGQGL | 91 |
| (heavy) | EWIGEINPSNGGGSNFNEKFKSKATLTVDKSSSTAYMQLSSLTSEDSA | |
| | VYYCTRGAFWGQGTLVTVSA | |
| 1009.1 | DVVMTQTPLTLSVTIGQPASISCKSSQSLLDSDRKTYLNWLLQRPGQ | 92 |
| (light) | SPKRLIYLVSKLDSGVPDRFTGSGSGTDFTLKISRVEAEDLGVYYCW | |
| | QVTHFPHTFGAGTKLELK | |
| 207.1 | QVTLKESGPGILKPSQTLSLTCSFSGFSLSTSGMGVGWIRQPSGKG | 93 |
| (heavy) | LEWLAHIWWDDDKYYNPSLKNRLTISKDTSRNQVFLKITSVDTADTA | |
| | TYYCVRSIYYYDSSPYYYVMDYWGQGTSVTVSS | |
| 207.1 | DIVMTQSHKFMSTSVGDRVSITCKASQDVSIAVAWYQQKPGQSPKL | 94 |
| (light) | LIYSASYRNTGVPDRFTGSGSGTDFTFTISSVQAEDLAVYYCQQHYS | |
| | SPLTFGAGTKLELK | |

TABLE 11
Sequences of CDRs of selected antibodies.

| | | | | SEQ ID |
|---|---|---|---|---|
| Antibody | Chain | CDR | Sequence | NO. |
| 2C6.1 | Heavy | CDR-H1 | GFNIKDTH | 95 |
| 2C6.1 | | CDR-H2 | IDPSNGNT | 96 |
| 2C6.1 | | CDR-H3 | ATGFAY | 97 |
| 2C6.1 | Light | CDR-L1 | GNIHNY | 98 |
| 2C6.1 | | CDR-L2 | NAK | 99 |
| 2C6.1 | | CDR-L3 | QHFWYTPWT | 100 |
| 8E11.1 | Heavy | CDR-H1 | GYAFSSYW | 101 |
| 8E11.1 | | CDR-H2 | TYPGDGDT | 102 |
| 8E11.1 | | CDR-H3 | ASQIYDGYYTFTY | 103 |
| 8E11.1 | Light | CDR-L1 | QSLLNSRTRKNY | 104 |
| 8E11.1 | | CDR-L2 | WAS | 105 |
| 8E11.1 | | CDR-L3 | KQSYNLWT | 106 |
| 12011.1 | Heavy | CDR-H1 | GYTFTNYG | 107 |
| 12011.1 | | CDR-H2 | INTYSGEP | 108 |
| 12011.1 | | CDR-H3 | ARSPGAYYTLDY | 109 |
| 12011.1 | Light | CDR-L1 | QSLLNSRTRKNY | 110 |
| 12011.1 | | CDR-L2 | WAS | 111 |
| 12011.1 | | CDR-L3 | KQSYNLYT | 112 |
| 9012.1 | Heavy | CDR-H1 | GYTFTNYG | 113 |
| 9012.1 | | CDR-H2 | INTYTGEP | 114 |
| 9012.1 | | CDR-H3 | GRGIRDYYTMDY | 115 |
| 9012.1 | Light | CDR-L1 | QSLLNNRTRKNY | 116 |
| 9012.1 | | CDR-L2 | WAS | 117 |
| 9012.1 | | CDR-L3 | KQSYNLYT | 118 |
| 9E4.1 | Heavy | CDR-H1 | GYSITSDYA | 119 |
| 9E4.1 | | CDR-H2 | ISYSGST | 120 |
| 9E4.1 | | CDR-H3 | AAYYRYGLAYFAY | 121 |
| 9E4.1 | Light | CDR-L1 | QSLLDSDGKTY | 122 |
| 9E4.1 | | CDR-L2 | LVS | 123 |
| 9E4.1 | | CDR-L3 | WQGTHFPQT | 124 |
| 10B10.1 | Heavy | CDR-H1 | GYTFTNFG | 125 |
| 10B10.1 | | CDR-H2 | INTFTGEP | 126 |
| 10B10.1 | | CDR-H3 | ARSPGRVYTLDY | 127 |
| 10B10.1 | Light | CDR-L1 | QSLLNSRTRKNY | 128 |
| 10B10.1 | | CDR-L2 | WAS | 129 |
| 10B10.1 | | CDR-L3 | KQSYNLYT | 130 |
| 1009.1 | Heavy | CDR-H1 | GYRFTSYW | 131 |
| 1009.1 | | CDR-H2 | IYPGNSDT | 132 |
| 1009.1 | | CDR-H3 | TRPYFDS | 133 |
| 1009.1 | Light | CDR-L1 | QSLLDSDGKTY | 134 |
| 1009.1 | | CDR-L2 | LVS | 135 |
| 1009.1 | | CDR-L3 | WQGTHFPQT | 136 |
| 1004.1 | Heavy | CDR-H1 | GFSITSDYA | 137 |
| 1004.1 | | CDR-H2 | IRYSGNT | 138 |
| 1004.1 | | CDR-H3 | ASTLEDSYWYFDV | 139 |
| 1004.1 | Light | CDR-L1 | QSIVHTNGNTY | 140 |
| 1004.1 | | CDR-L2 | KVS | 141 |
| 1004.1 | | CDR-L3 | FQGSHVPLT | 142 |
| 1009.1 | Heavy | CDR-H1 | GYTFTSYY | 143 |
| 1009.1 | | CDR-H2 | INPSNGGS | 144 |
| 1009.1 | | CDR-H3 | TRGAF | 145 |
| 1009.1 | Light | CDR-L1 | QSLLDSDRKTY | 146 |
| 1009.1 | | CDR-L2 | LVS | 147 |
| 1009.1 | | CDR-L3 | WQVTHFPHT | 148 |
| 207.1 | Heavy | CDR-H1 | GFSLSTSGMG | 149 |
| 207.1 | | CDR-H2 | IWWDDDK | 150 |
| 207.1 | | CDR-H3 | VRSIYYYDSSPYYYVMDY | 151 |
| 207.1 | Light | CDR-L1 | QDVSIA | 152 |
| 207.1 | | CDR-L2 | SAS | 153 |
| 207.1 | | CDR-L3 | QQHYSSPLT | 154 |

The sequencing data shows unique heavy and light pairings have been generated for the 10 hybridoma clones sequenced. The following clones were found to share the same light chain: 12D11.1 (raised against 3,1 cyclic peptide) and 10.B310.1 (raised against 3,2 cyclic peptide); and 9E4.1 and 1009.1 (both raised to 3,2 cyclic peptide).

The nucleic acid sequences encoding the heavy chain and light variable domains of different antibodies are provided below.

TABLE 12
Nucleic Acid sequences for antibody clones

| Clone | Heavy | Light |
|---|---|---|
| (chain) | variable chain | variable chain |
| 2C6 | 155 | 156 |
| 8E11 | 157 | 158 |
| 12D11 | 159 | 160 |
| 9D12 | 161 | 162 |
| 9E4 | 163 | 164 |
| 10B10 | 165 | 166 |
| 10C9 | 167 | 168 |
| 10D4 | 169 | 170 |
| 10D9 | 171 | 172 |
| 2C7 | 173 | 174 |

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

1. A cyclic compound, comprising: a tau peptide comprising at least 4 residues of KLDFK (SEQ ID NO: 1), optionally KLDF (SEQ ID NO:2), LDFK (SEQ ID NO: 3) or KLDFK (SEQ ID NO: 1); and a linker, wherein the linker is covalently coupled to the peptide N-terminus residue and the C-terminus residue.

2. (canceled)

3. (canceled)

4. (canceled)

5. The cyclic compound of claim 1, wherein the linker comprises or consists of 1-8 amino acids and/or one or more functionalizable moieties, optionally wherein the linker amino acids are selected from alanine (A) or glycine (G) and/or wherein the functionalizable moiety is cysteine (C).

6. (canceled)

7. The cyclic compound of claim 1, wherein the linker comprises or consists of GGCG (SEQ ID NO: 186 1,2 linker), GCGG (SEQ ID NO: 43 2,1), -GCG (1,1 linker), GCGGG (SEQ ID NO:44; 3,1 linker), GGCGGG (SEQ ID NO: 45; 3,2 linker), GGGCG (SEQ ID NO: 46; 1,3 linker), GGGGCGG (SEQ ID NO: 65; 2,4 linker) or GCGGGG (SEQ ID NO: 47; 4,1 linker.

8. (canceled)

9. The cyclic compound of claim 1, wherein the cyclic compound is selected from a cyclic compound recited in Table 2 or 4, optionally wherein the cyclic compound is selected from cyclo(CGGKLDFKG) (SEQ ID NO: 31; with linker 2,1), cyclo(CGKLDFKG) (SEQ ID NO: 27 with linker 1,1), cyclo(CGGGGKLDFKG) (SEQ ID NO:39; with linker 4,1), cyclo(CGKLDFKGG) (SEQ ID NO: 28; with linker 1,2), cyclo(CGGKLDFKGGGG) (SEQ ID NO: 34; with 3,2 linker), cyclo(CGGGKLDFKG) (SEQ ID NO: 35; with linker 3,1), cyclo(CGKLDFG) (SEQ ID NO: 7; with linker 1,1), cyclo(CGGGKLDFG) (SEQ ID NO: 15; with linker 3,1), cyclo(CGGGGKLDFG) (SEQ ID NO: 19; with linker 4,1), cyclo(CGGGKLDFGG) (SEQ ID NO: 16 with linker 3,2), cyclo(CGGLDFKG) (SEQ ID NO: 52; with linker 2,1) or cyclo(CGLDFKGG) (SEQ ID NO: 49; with linker 1, 2).

10. An immunogen comprising the cyclic compound of claim 1, optionally wherein the immunogen is coupled to a carrier protein or immunogenicity enhancing agent and/or is a multiantigenic peptide (MAP), optionally wherein the carrier protein is bovine serum albumin (BSA) or the immunogenicity-enhancing agent is keyhole limpet haemocyanin (KLH).

11. (canceled)

12. (canceled)

13. A composition comprising the cyclic compound of claim 1 or an immunogen comprising the cyclic compound of claim 1 and optionally a diluent.

14. The composition of claim 13, comprising an adjuvant, optionally wherein the adjuvant comprises aluminum phosphate or aluminum hydroxide, aluminum sulfate.

15. (canceled)

16. An antibody that selectively binds an epitope in the tau peptide in the cyclic compound of claim 1 compared to a corresponding linear compound and/or tau monomer and/or raised using an immunogen comprising the cyclic compound or a composition comprising the cyclic compound of 15, optionally wherein the antibody is at least 1.5 fold, at least 2 fold, at least 2.5 fold, at least 3 fold, at least 3.5 fold or at least 4 fold more selective for the cyclic compound compared to the corresponding linear compound and/or wherein the antibody selectively binds misfolded oligomeric tau polypeptide and/or soluble fibrils compared to monomeric tau polypeptide and/or microtubule-bound tau polypeptide, optionally wherein the antibody is at least 1.5 fold, at least 2 fold, at least 2.5 fold, at least 3 fold, at least 3.5 fold or at least 4 fold more selective for the misfolded oligomeric tau polypeptide and/or soluble fibrils compared to the monomeric tau polypeptide and/or microtubule-bound tau polypeptide.

17. (canceled)

18. (canceled)

19. (canceled)

20. (canceled)

21. (canceled)

22. (canceled)

23. The antibody of claim 16, comprising a light chain variable region and a heavy chain variable region, the heavy chain variable region comprising complimentary determining regions CDR-H1, CDR-H2 and CDR-H3, the light chain variable region comprising complementarity determining regions CDR-L1, CDR-L2 and CDR-L3 and with the amino acid sequences of said CDRs comprising the sequences:
CDR-H1:
SEQ ID NO: 95
GFNIKDTH;
CDR-H2:
SEQ ID NO: 96
IDPSNGNT;
CDR-H3:
SEQ ID NO: 97
ATGFAY;
CDR-L1:
SEQ ID NO: 98
GNIHNY;
CDR-L2:
SEQ ID NO: 99
NAK;
and
CDR-L3:
SEQ ID NO: 100
QHFVVYTPVVT;
CDR-H1:
SEQ ID NO: 101
GYAFSSYW;
CDR-H2:
SEQ ID NO: 102
IYPGDGDT;
CDR-H3:
SEQ ID NO: 103
ASQIYDGYYTFTY;
CDR-L1:
SEQ ID NO: 104
QSLLNSRTRKNY;
CDR-L2:
SEQ ID NO: 105
WAS;
and
CDR-L3:
SEQ ID NO: 106
KQSYNLVVT;
CDR-H1:
SEQ ID NO: 107
GYTFTNYG;
CDR-H2:
SEQ ID NO: 108
INTYSGEP;
CDR-H3:
SEQ ID NO: 109
ARSPGAYYTLDY;
CDR-L1:
SEQ ID NO: 110
QSLLNSRTRKNY;
CDR-L2:
SEQ ID NO: 111
WAS;
and
CDR-L3:
SEQ ID NO: 112
KQSYNLYT;
CDR-H1:
SEQ ID NO: 113
GYTFTNYG;
CDR-H2:
SEQ ID NO: 114
INTYTGEP;
CDR-H3:
SEQ ID NO: 115
GRGIRDYYTMDY;
CDR-L1:
SEQ ID NO: 116
QSLLNNRTRKNY;
CDR-L2:
SEQ ID NO: 117
WAS;
and
CDR-L3:
SEQ ID NO: 118
KQSYNLYT;
CDR-H1:
SEQ ID NO: 119
GYSITSDYA;
CDR-H2:
SEQ ID NO: 120
ISYSGST;
CDR-H3:
SEQ ID NO: 121
AAYYRYGLAYFAY;
CDR-L1:
SEQ ID NO: 122
QSLLDSDGKTY;
CDR-L2:
SEQ ID NO: 123
LVS;
and
CDR-L3:
SEQ ID NO: 124
WQGTHFPQT;
CDR-H1:
SEQ ID NO: 125
GYTFTNFG;
CDR-H2:
SEQ ID NO: 126
INTFTGEP;
CDR-H3:
SEQ ID NO: 127
ARSPGRVYTLDY;
CDR-L1:
SEQ ID NO: 128
QSLLNSRTRKNY;
CDR-L2:
SEQ ID NO: 129
WAS;
and
CDR-L3:
SEQ ID NO: 130
KQSYNLYT;
CDR-H1:
SEQ ID NO: 131
GYRFTSYW;
CDR-H2:
SEQ ID NO: 132
IYPGNSDT;
CDR-H3:
SEQ ID NO: 133
TRPYFDS;
CDR-L1:
SEQ ID NO: 134
QSLLDSDGKTY;
CDR-L2:
SEQ ID NO: 135
LVS;
and
CDR-L3:
SEQ ID NO: 136
WQGTHFPQT;
CDR-H1:
SEQ ID NO: 137
GFSITSDYA;
CDR-H2:
SEQ ID NO: 138
IRYSGNT;
CDR-H3:
SEQ ID NO: 139
ASTLEDSYVVYFDV;
CDR-L1:
SEQ ID NO: 140
QSIVHTNGNTY;
CDR-L2:
SEQ ID NO: 141
KVS;
and
CDR-L3:
SEQ ID NO: 142
FQGSHVPLT;
CDR-H1:
SEQ ID NO: 143
GYTFTSYY;
CDR-H2:
SEQ ID NO: 144
INPSNGGS;
CDR-H3:
SEQ ID NO: 145
TRGAF;
CDR-L1:
SEQ ID NO: 146
QSLLDSDRKTY;
CDR-L2:
SEQ ID NO: 147
LVS;
and
CDR-L3:
SEQ ID NO: 148
WQVTHFPHT;
or
CDR-H1:
SEQ ID NO: 149
GFSLSTSGMG;
CDR-H2:
SEQ ID NO: 150
IVVWDDDK;
CDR-H3:
SEQ ID NO: 151
VRSIYYYDSSPYYYVMDY;
CDR-L1:
SEQ ID NO: 152
QDVSIA;
CDR-L2:
SEQ ID NO: 153
SAS;
and
CDR-L3:
SEQ ID NO: 154
QQHYSSPLT.

24. The antibody of claim 23, wherein the antibody comprises:
a) a heavy chain variable region comprising: i) an amino acid sequence as set forth in SEQ ID NO: 75, ii) an amino acid sequence with at least 80%, at least 90%, or at least 95% sequence identity to SEQ ID NO: 75, wherein the CDR sequences are as set forth in SEQ ID NOs: 95-97, or iii) a conservatively substituted amino acid sequence of i), and/or wherein the antibody comprises a light chain variable region comprising an amino acid sequence as set forth in SEQ ID NO: 76, ii) an amino acid sequence with at least 80%, at least 90%, or at least 95% sequence identity to SEQ ID NO: 76, wherein the CDR sequences are as set forth in SEQ ID NOs: 98-100, or iii) a conservatively substituted amino acid sequence of i);
b) a heavy chain variable region comprising: i) an amino acid sequence as set forth in SEQ ID NO: 77, ii) an amino acid sequence with at least 80%, at least 90%, or at least 95% sequence identity to SEQ ID NO: 77, wherein the CDR sequences are as set forth in SEQ ID NOs: 101-103, or iii) a conservatively substituted amino acid sequence of i), and/or wherein the antibody comprises a light chain variable region comprising an amino acid sequence as set forth in SEQ ID NO: 78, ii) an amino acid sequence with at least 80%, at least 90%, or at least 95% sequence identity to SEQ ID NO: 78, wherein the CDR sequences are as set forth in SEQ ID NOs: 104-106, or iii) a conservatively substituted amino acid sequence of i);
c) heavy chain variable region comprising: i) an amino acid sequence as set forth in SEQ ID NO: 79, ii) an amino acid sequence with at least 80%, at least 90%, or at least 95% sequence identity to SEQ ID NO: 79, wherein the CDR sequences are as set forth in SEQ ID NOs: 107-109, or iii) a conservatively substituted amino acid sequence of i), and/or wherein the antibody comprises a light chain variable region comprising an amino acid sequence as set forth in SEQ ID NO: 80, ii) an amino acid sequence with at least 80%, at least 90%, or at least 95% sequence identity to SEQ ID NO: 80, wherein the CDR sequences are as set forth in SEQ ID NOs: 110-112, or iii) a conservatively substituted amino acid sequence of i);
d) a heavy chain variable region comprising: i) an amino acid sequence as set forth in SEQ ID NO: 81, ii) an amino acid sequence with at least 80%, at least 90%, or at least 95% sequence identity to SEQ ID NO: 81, wherein the CDR sequences are as set forth in SEQ ID NOs: 113-115, or iii) a conservatively substituted amino acid sequence of i), and/or wherein the antibody comprises a light chain variable region comprising an amino acid sequence as set forth in SEQ ID NO: 82, ii) an amino acid sequence with at least 80%, at least 90%, or at least 95% sequence identity to SEQ ID NO: 82, wherein the CDR sequences are as set forth in SEQ ID NOs: 116-118, or iii) a conservatively substituted amino acid sequence of i);
e) a heavy chain variable region comprising: i) an amino acid sequence as set forth in SEQ ID NO: 83, ii) an amino acid sequence with at least 80%, at least 90%, or at least 95% sequence identity to SEQ ID NO: 83, wherein the CDR sequences are as set forth in SEQ ID NOs: 119-121, or iii) a conservatively substituted amino acid sequence of i), and/or wherein the antibody comprises a light chain variable region comprising an amino acid sequence as set forth in SEQ ID NO: 84, ii) an amino acid sequence with at least 80%, at least 90%, or at least 95% sequence identity to SEQ ID NO: 84, wherein the CDR sequences are as set forth in SEQ ID NOs: 122-124, or iii) a conservatively substituted amino acid sequence of i);
f) a heavy chain variable region comprising: i) an amino acid sequence as set forth in SEQ ID NO: 85, ii) an amino acid sequence with at least 80%, at least 90%, or at least 95% sequence identity to SEQ ID NO: 85, wherein the CDR sequences are as set forth in SEQ ID NOs: 125-127, or iii) a conservatively substituted amino acid sequence of i), and/or wherein the antibody comprises a light chain variable region comprising an amino acid sequence as set forth in SEQ ID NO: 86, ii) an amino acid sequence with at least 80%, at least 90%, or at least 95% sequence identity to SEQ ID NO: 86, wherein the CDR sequences are as set forth in SEQ ID NOs: 128-130, or iii) a conservatively substituted amino acid sequence of i);
g) a heavy chain variable region comprising: i) an amino acid sequence as set forth in SEQ ID NO: 87, ii) an amino acid sequence with at least 80%, at least 90%, or at least 95% sequence identity to SEQ ID NO: 87, wherein the CDR sequences are as set forth in SEQ ID NOs: 131-133, or iii) a conservatively substituted amino acid sequence of i), and/or wherein the antibody comprises a light chain variable region comprising an amino acid sequence as set forth in SEQ ID NO: 88, ii) an amino acid sequence with at least 80%, at least 90%, or at least 95% sequence identity to SEQ ID NO: 88, wherein the CDR sequences are as set forth in SEQ ID NOs: 134-136, or iii) a conservatively substituted amino acid sequence of i);
h) a heavy chain variable region comprising: i) an amino acid sequence as set forth in SEQ ID NO: 89, ii) an amino acid sequence with at least 80%, at least 90%, or at least 95% sequence identity to SEQ ID NO: 89, wherein the CDR sequences are as set forth in SEQ ID NOs: 137-139, or iii) a conservatively substituted amino acid sequence of i), and/or wherein the antibody comprises a light chain variable region comprising an amino acid sequence as set forth in SEQ ID NO: 90, ii) an amino acid sequence with at least 80%, at least 90%, or at least 95% sequence identity to SEQ ID NO: 90, wherein the CDR sequences are as set forth in SEQ ID NOs: 140-142, or iii) a conservatively substituted amino acid sequence of i);
i) a heavy chain variable region comprising: i) an amino acid sequence as set forth in SEQ ID NO: 91, ii) an amino acid sequence with at least 80%, at least 90%, or at least 95% sequence identity to SEQ ID NO: 91, wherein the CDR sequences are as set forth in SEQ ID NOs: 143-145, or iii) a conservatively substituted amino acid sequence of i), and/or wherein the antibody comprises a light chain variable region comprising an amino acid sequence as set forth in SEQ ID NO: 92, ii) an amino acid sequence with at least 80%, at least 90%, or at least 95% sequence identity to SEQ ID NO: 92, wherein the CDR sequences are as set forth in SEQ ID NOs: 146-148, or iii) a conservatively substituted amino acid sequence of i); or
j) a heavy chain variable region comprising: i) an amino acid sequence as set forth in SEQ ID NO: 93, ii) an amino acid sequence with at least 80%, at least 90%, or at least 95% sequence identity to SEQ ID NO: 93, wherein the CDR sequences are as set forth in SEQ ID NOs: 149-151, or iii) a conservatively substituted amino acid sequence of i), and/or wherein the antibody comprises a light chain variable region comprising an amino acid sequence as set forth in SEQ ID NO: 94, ii) an amino acid sequence with at least 80%, at least 90%, or at least 95% sequence identity to SEQ ID NO: 94, wherein the CDR sequences are as set forth in SEQ ID NOs: 152-154, or iii) a conservatively substituted amino acid sequence of i).

25. (canceled)

26. (canceled)

27. (canceled)

28. (canceled)

29. (canceled)

30. (canceled)

31. (canceled)

32. (canceled)

33. (canceled)

34. An immunoconjugate comprising the antibody, antibody heavy chain variable domain, or antibody light chain variable domain of claim 23 and a detectable label.

35. A nucleic acid encoding the antibody, antibody heavy chain variable domain, or antibody light chain variable domain of claim 23, or a vector comprising the nucleic acid.

36. The nucleic acid of claim 35, wherein the nucleic acid i) encodes a heavy chain variable domain, the nucleic acid encoding heavy chain variable domain comprising the sequence of any one of SEQ ID NOs: 155, 157, 159, 161, 163, 165, 167, 169, or 173, or a sequence with at least 70%, 80%, 85%, 90%, 95%, 98% or 99% sequence identity to any one of SEQ ID Nos: 155, 157, 159, 161, 163, 165, 167, 169, or 173; ii) encodes a light chain variable domain, the nucleic acid encoding light chain variable domain comprising the sequence of any one of SEQ ID NOs: 156, 158, 160, 162, 164, 166, 168, 170, 172, or 174, or a sequence with at least 70%, 80%, 85%, 90%, 95%, 98% or 99% sequence identity to any one of SEQ ID Nos: 156, 158, 160, 162, 164, 166, 168, 170, 172, or 174; or iii) encodes a heavy chain variable domain and a light chain variable domain, the nucleic acid encoding the heavy chain variable domain and light chain variable domain, respectively, comprising the sequences: SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 167 and 168; SEQ ID NOs: 169 and 170; SEQ ID NOs: 171 and 172; or SEQ ID NOs: 173 and 174, or sequences with at least 70%, 80%, 85%, 90%, 95%, 98% or 99% sequence identity to SEQ ID NOs: 155 and 156; SEQ ID NOs: 157 and 158; SEQ ID NOs: 159 and 160; SEQ ID NOs: 161 and 162; SEQ ID NOs: 163 and 164; SEQ ID NOs: 165 and 166; SEQ ID NOs: 167 and 168; SEQ ID NOs: 169 and 170; SEQ ID NOs: 171 and 172; or SEQ ID NOs: 173 and 174.

37. (canceled)

38. The vector of claim 35, wherein the vector is a viral vector, optionally, an adenoviral vector, an adenoviral-associated vector, or a retroviral vector, preferably a lentiviral vector.

39. A cell expressing the antibody, antibody heavy chain variable domain, or antibody light chain variable domain of claim 23, or a nucleic acid encoding the antibody, antibody heavy chain variable domain, or antibody light chain variable domain, or a vector comprising the nucleic acid.

40. The cell of claim 39, wherein the cell is selected from a mammalian cell, optionally a CHO cell or an HEK-293 cell, or an insect cell, optionally a Sf9 cell, Sf21 cell, Tni cell, or S2 cell.

41. A composition comprising:
a) the antibody, antibody heavy chain variable domain, or antibody light chain variable domain of claim 23;
b) an immunoconjugate comprising the antibody, antibody heavy chain variable domain, or antibody light chain variable domain and a detectable label;
c) a nucleic acid encoding the antibody, antibody heavy chain variable domain, or antibody light chain variable domain or a vector comprising the nucleic acid; or
d) a cell expressing the antibody, antibody heavy chain variable domain, or antibody light chain variable domain or comprising the nucleic acid or the vector;
optionally with a diluent.

42. A kit comprising:
a) the cyclic compound of claim 1;
b) an immunogen comprising the cyclic compound of;
c) an antibody, antibody heavy chain variable domain or antibody light chain variable domain that selectively binds an epitope in the tau peptide in the cyclic compound compared to a corresponding linear compound and/or tau monomer and/or raised using an immunogen comprising the cyclic compound;
d) an immunoconjugate comprising the and a detectable label;
e) a nucleic acid encoding the antibody, antibody heavy chain variable domain or antibody light chain variable domain, or a vector comprising the nucleic acid of; or
f) a cell expressing the antibody, antibody heavy chain variable domain or antibody light chain variable domain or comprising the nucleic acid or the vector.

43. A method of making an antibody, comprising administering an immunogenic form of the cyclic compound or an immunogen comprising the cyclic compound of claim 1 or a composition comprising the immunogenic form of the cyclic compound or the immunogen comprising the cyclic compound to a subject and isolating antibody and/or cells expressing antibody selective for the tau peptide in the cyclic compound or the immunogen administered, optionally testing the antibody to see if it selectively binds the cyclic compound compared to a corresponding linear peptide and/or misfolded oligomeric tau and/or soluble fibrils relative to monomeric tau polypeptide and/or microtubule-bound tau polypeptide.

44. A method of determining if a test sample contains misfolded oligomeric tau polypeptide the method comprising:
a. contacting the test sample with the antibody of claim 16 or an immunoconjugate comprising the antibody and a detectable label under conditions permissive for forming an antibody: misfolded oligomeric tau polypeptide complex and/or an antibody: soluble fibril complex; and
b. detecting the presence of any complex;
c. wherein the presence of detectable complex is indicative that the sample may contain misfolded oligomeric tau polypeptide.

45. The method of claim 44, wherein the test sample comprises brain tissue extract and/or cerebrospinal fluid (CSF), optionally wherein the test sample is a human sample and/or wherein detecting the complex comprises contacting the complex with a pan tau antibody.

46. (canceled)

47. (canceled)

48. A method of reducing or inhibiting tau aggregation/aggregates and/or propagation, comprising contacting a cell or tissue expressing misfolded oligomeric tau polypeptide and/or soluble fibrils, with an antibody of claim 16, an immunoconjugate comprising the antibody and a detectable label, a nucleic acid encoding the antibody, antibody heavy chain variable domain, or antibody light chain variable domain, or a vector comprising the nucleic acid, optionally wherein the cell or tissue is in vivo in a subject.

49. (canceled)

50. A method of treating a tauopathy in a subject in need thereof, comprising administering to the subject an effective amount of the antibody, antibody heavy chain variable domain, or antibody light chain variable domain of claim 16 or a composition comprising said antibody, an immunoconjugate comprising the antibody and a detectable label, a nucleic acid encoding the antibody, antibody heavy chain variable domain, or antibody light chain variable domain, or a vector comprising the nucleic acid.

51. The method of claim 50, wherein the tauopathy is selected from Alzheimer's disease (AD), Pick's disease, frontotemporal dementia or frontotemporal lobar degeneration, progressive supranuclear palsy, corticobasal degeneration, primary age-related tauopathy, chronic traumatic encephalopathy, subacute sclerosing panencephalitis, frontotemporal dementia or parkinsonism linked to chromosome 17.
